SEQUENCE LISTING (19 sequences)

SEQ ID NO: 1 - DNA, 4154 bp, Homo sapiens
  CDS (167)..(3607)
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 2 - protein, 1147 aa, Homo sapiens [sequence omitted]

SEQ ID NO: 3 - DNA, 4154 bp, Homo sapiens
  CDS (167)..(3607)
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcatgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 4 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGMKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGASSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 5 - DNA, 4154 bp, Homo sapiens
  CDS (167)..(3607)
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccatgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 6 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAMYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGASSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 7 - DNA, 4154 bp, Homo sapiens
  CDS (167)..(3607)
taacgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcatgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccatgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcgccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttata

SEQ ID NO: 8 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGMKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAMYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGASSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 9 - DNA, 24 bp, Homo sapiens
cctcagtcttcacgccctccatga

SEQ ID NO: 10 - DNA, 25 bp, Homo sapiens
ccacctcagtcttcatgccctccat

SEQ ID NO: 11 - DNA, 19 bp, Homo sapiens
ctctgttcctttcggccca

SEQ ID NO: 12 - DNA, 19 bp, Homo sapiens
aaaggtgagccagcatccg

SEQ ID NO: 13 - DNA, 24 bp, Homo sapiens
actcaggccccgccgtgtacttct

SEQ ID NO: 14 - DNA, 25 bp, Homo sapiens
cactcaggccccgccatgtacttct

SEQ ID NO: 15 - DNA, 21 bp, Homo sapiens
ttgtctacggtgagccagtca

SEQ ID NO: 16 - DNA, 18 bp, Homo sapiens
aggtcagcctgcttgcga

SEQ ID NO: 17 - DNA, 24 bp, Homo sapiens
agcggataacaatttcacacagga

SEQ ID NO: 18 - DNA, 24 bp, Homo sapiens
cgccagggttttcccagtcacgac

SEQ ID NO: 19 - DNA, 27 bp, Homo sapiens
gccctccatgagcacgtctgtgttccc

(19) United States
(12) Patent Application Publication
(10) Pub. No.: US 2005/0014177 A1
(43) Pub. Date: Jan. 20, 2005

(54) Human sterol response element binding protein 1 (SREBP1) single nucleotide polymorphisms

(76) Inventors: Koustubh Ranade, Princeton, NJ (US); Terrye Delmonte, Lawrenceville, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/848,723

(22) Filed: May 19, 2004

Related U.S. Application Data
(60) Provisional application No. 60/471,673, filed on May 19, 2003.

(51) Int. Cl. C12Q 1/68; C07H 21/04; C07K 14/72

(52) U.S. Cl. 435/6; 435/69.1; 435/320.1; 435/325; 530/358; 536/23.5

(57) ABSTRACT

The present invention provides polynucleotides and polypeptides corresponding to novel gene sequences associated with cardiovascular disorders. The present invention also provides polynucleotide fragments corresponding to the genomic and/or coding regions of these genes that comprise at least one polymorphic site per fragment. Allele-specific primers and probes that hybridize to these regions, and/or which comprise at least one polymorphic site are also provided. The polynucleotides, primers, and probes of the present invention are useful in phenotype correlations, paternity testing, medicine, and genetic analysis. Also provided are vectors, host cells, antibodies, and recombinant and synthetic methods for producing the polypeptides of the present invention. The present invention further relates to diagnostic and therapeutic methods for applying these novel polypeptides to the diagnosis, treatment, and/or prevention of various diseases and/or disorders, particularly cardiovascular diseases related to these polypeptides. The present invention further relates to screening methods for identifying agonists and antagonists of the polynucleotides and polypeptides of the present invention.

This application claims the benefit of U.S. Provisional Patent Application Ser. No. 60/471,673 filed May 19, 2003. The entire teachings of the referenced application are incorporated herein by reference.

### FIELD OF THE INVENTION

In one aspect, the present invention provides polynucleotides and polypeptides corresponding to novel gene sequences associated with cardiovascular disease (CVD) in general and more particularly with plasma high density lipid (HDL) levels in a subject. The invention also provides polynucleotide fragments corresponding to the genomic and/or coding regions of these genes comprising at least one polymorphic site per fragment. Allele-specific primers and probes that hybridize to these regions, and/or that comprise at least one polymorphic site are also provided. The polynucleotides, primers, and probes of the present invention can be employed, for example, in phenotype correlations, paternity testing, medicine, and genetic analysis. In another aspect, the invention further relates to in diagnostic, prognostic, ameliorative capacities and/or therapeutic methods for applying these novel polypeptides to the prediction, diagnosis, treatment, and/or prevention of various diseases and/or disorders, particularly cardiovascular diseases and/or HDL-related diseases associated with these polypeptides. In yet another aspect, the invention further relates to screening methods for identifying agonists and antagonists of the polynucleotides and polypeptides of the present invention. Also provided are vectors, host cells, antibodies, and recombinant and synthetic methods for producing the polypeptides.

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |

Amino Acid Abbreviations

| Single-Letter Code | Three-Letter Code | Name |
|---|---|---|
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| Amino Acid | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic Acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND OF THE INVENTION

The genomes of all organisms undergo spontaneous mutation in the course of their continuing evolution, generating variant forms of progenitor nucleic acid sequences (Gusella, (1986) Ann. Rev. Biochem. 55:831-854). The variant form can confer an evolutionary advantage or disadvantage relative to a progenitor form, or can be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both the progenitor and variant form(s) survive and co-exist in a species population. The coexistence of multiple forms of a sequence gives rise to polymorphisms.

Several different types of polymorphisms have been reported. For example, a restriction fragment length polymorphism (RFLP) is a variation in DNA sequence that alters the length of a restriction fragment (Botstein et al., (1980) Am. J. Hum. Genet. 32:314-331). The restriction fragment length polymorphism can create or delete a restriction site, thus changing the length of the restriction fragment. RFLPs have been used in human and animal genetic analyses (see, e.g., PCT Publications WO 90/13668 and WO 90/11369; Donis-Keller, (1987) Cell 51:319-337; Lander et al., (1989) Genetics 121:85-99). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as “variable number tandem repeat” (VNTR) polymorphisms. VNTRs have been used in identity and paternity analysis (see, e.g., Annour et al., (1992) FEBS Lett. 307:113-115; U.S. Pat. No. 5,075,217; PCT Publication WO 91/14003; EP 370,719), and in a large number of genetic mapping studies.

Yet other polymorphisms take the form of single nucleotide variations between individuals of the same species. Such polymorphisms are far more frequent than RFLPs, STRs and VNTRs. Some single nucleotide polymorphisms (SNP) occur in protein-coding nucleic acid sequences, referred to as coding sequence SNPs (cSNPs). In these cases, one of the polymorphic forms can give rise to the expression of a defective or otherwise variant protein and, potentially, a genetic disease condition. Examples of genes in which polymorphisms within coding sequences give rise to genetic disease include hemoglobin S (β^{S}; sickle cell anemia), apoE4 (Alzheimer's Disease), Factor V Leiden (thrombosis), and CFTR (cystic fibrosis). cSNPs can alter the codon sequence of the gene and therefore specify an alternative amino acid. Such changes are called “missense” when another amino acid is substituted and “nonsense” when the alternative codon specifies a stop signal in protein translation. When the cSNP does not alter the amino acid specified the cSNP is referred to as “silent”.

Other single nucleotide polymorphisms occur in noncoding regions. Some of these polymorphisms can also result in defective protein expression (e.g., as a result of defective splicing). Still other single nucleotide polymorphisms have no phenotypic effects. Single nucleotide polymorphisms can be employed in the same manner RFLPs and VNTRs can be employed, but offer several advantages.

Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The different forms of characterized single nucleotide polymorphisms are sometimes easier to distinguish than other types of polymorphism (e.g., by the use of assays employing allele-specific hybridization probes or primers).

Only a small percentage of the total repository of polymorphisms in humans and other organisms has been identified. The limited number of polymorphisms identified to date is due, in part, to the large amount of work required to detect the polymorphisms by conventional methods. For example, one conventional approach for identifying polymorphisms is to sequence the same stretch of DNA in a population of individuals by dideoxy sequencing. In this approach, the amount of work required to identify the polymorphism increases in proportion to both the length of sequence and the number of individuals in a population; thus, such techniques become impractical for large stretches of DNA or large numbers of persons.

Cardiovascular disease is the number one killer in the United States, and atherosclerosis is the major cause of heart disease and stroke (“Heart and Stroke Statistical Update”, American Heart Association, Dallas, Tex., USA, 1997). It is known that cholesterol plays an important role in atherogenesis (Kannel et al., (1971). Ann. Intern. Med. 74:1-12). In mammals, most cholesterol serves as a structural element in the walls of cells, whereas the rest either serves as the starting material for the synthesis of bile acids, steroid hormones and vitamin D or is in transit in the blood to a given location.

The transport of cholesterol and other lipids through the circulatory system is mediated by their packaging into lipoprotein carriers. These spherical particles comprise protein and phospholipid shells surrounding a core of neutral lipid, including unesterified or esterified cholesterol and triglycerides.

It is known that the risk of atherosclerosis increases with increasing concentrations of low-density lipoprotein (LDL) cholesterol. In contrast, risk of cardiovascular disease is inversely related to concentrations of high-density lipoprotein (HDL) cholesterol (Gordon et al., (1977) Am. J. Med. 62:707-14).

Although cholesterol plays a crucial role in the etiology of atherosclerosis, numerous other genetic and environmental factors are known to contribute to the disease. For example, non-modifiable factors that influence disease pre-disposition include age (risk increases with age), gender (men are more prone than are pre-menopausal women) and family history (i.e., genetics). Modifiable factors that increase risk of disease include obesity, blood pressure, diet, exercise, poor control of diabetes, alcohol intake and smoking. Thus, the risk of atherosclerosis is determined by a complex interplay of genetic and environmental risk factors (Pearson, (2002) Circulation 105:886-892). While there is considerable knowledge of the environmental risk factors, present understanding of the genetics of atherosclerosis is poor.

Because LDL and HDL have opposing and significant effects on the risk of atherosclerosis, understanding the genetic contribution to levels of these lipoproteins in the blood not only will enhance the understanding of atherosclerosis but can also facilitate identification of at-risk individuals, even before abnormalities in their plasma HDL and LDL levels become apparent. Indeed, mutations in the LDL receptor gene result in familial hypercholesterolemia, a disease characterized by elevated plasma LDL levels and premature atherosclerosis (Goldstein & Brown, in The Metabolic Basis of Inherited Disease, 6th ed., (Scriver, Beaudet, Sly and Valle, eds.), McGraw-Hill, New York, N.Y., USA, (1989) pp. 1215-1250). Similarly, mutations in the ATP-binding cassette 1 (ABC1) gene cause Tangier disease, a disorder that is characterized by the near-absence of HDL in plasma, peripheral neuropathy and premature heart disease (Bodzioch et al., (1999 Nature Genet. 22:347-351). Thus, genes that influence plasma HDL and LDL levels can serve as targets for drugs that modulate lipid levels and therefore the risk of atherosclerosis. In fact, drugs known as “statins” inhibit the enzyme, 3-hydroxy-3-methylglutaryl-CoA-reductase (HMG CoA reductase), and thereby reduce both levels of plasma LDL levels and risk of atherosclerosis (Sacks et al., (1996) N. Engl. J. Med. 335:1001-1009). Analogous drugs that significantly increase plasma HDL levels are not presently known.

A family of transcription factors called Sterol Response Element Binding Proteins (SREBPs) regulates lipid levels in mammals. It is known that genes called Sterol Response Element Binding Protein 1 (SREBP1) and called Sterol Response Element Binding Protein 2 (SREBP2) directly activate the expression of over thirty different genes implicated in the synthesis and uptake of cholesterol, fatty acids, triglycerides, phospholipids and the NADPH co-factor required to synthesize these molecules (Horton et al., (2001) J. Clin. Invest. 109:1125-1131). Further, it is known that the expression of both the LDL receptor and HDL receptor genes is regulated by SREBPs (Smith et al., (1990) J. Biol. Chem. 265: 2306-2310; Lopez & McLean, (1999) Endocrinol. 140:5669-5681). Thus, the inter-individual variation in plasma HDL and LDL levels (and therefore variation in risk of atherosclerosis) seen in the general population can be attributed, at least in part, to genetic variation in SREBPs.

SNPs that result in missense changes in the SREBP1 gene form aspects of the present invention. In one aspect, these SNPs result in valine-to-methionine changes in SREBP1, at least at amino acid residue 417 and at residue 580. In another aspect of the present invention, these polymorphisms are associated with elevated plasma HDL levels.

There is a range of applications in which the present invention can be employed. For example, since plasma HDL levels are inversely related to risk of atherosclerosis, identifying the presence (or absence) of one or more SNPs (e.g., a valine-to-methionine SNP) can facilitate identification of individuals predisposed to (or protected from) heart disease, even before their plasma HDL levels are considered abnormal. In another example, the SREBP1 gene can serve as a target for modulation of plasma HDL levels in a manner in which HMG CoA reductase can serve as a target for modulating LDL levels. Further, recent evidence implicates SREBP1 in the etiology of metabolic abnormalities seen in HIV-infected people treated with protease-inhibitors (Bastard et al., (2002) Lancet 359:1026-1031). Thus, the presence or absence of these SNPs can also be useful in predicting susceptibility of lipid-related side-effects associated with protease-inhibitors in a subject.

### SUMMARY OF THE INVENTION

In one embodiment, the present invention pertains to single nucleotide polymorphisms associated with elevated plasma HDL levels and/or that can protect an individual from cardiovascular disease. In one embodiment, the present invention comprises an isolated nucleic acid molecule derived from a human gene encoding a SREBP1 protein, wherein the nucleic acid molecule is selected from the group consisting of: (a) a nucleic acid derived from a human gene encoding a SREBP1 protein comprising at least one polymorphic position; and (b) a nucleic acid that hybridizes to a nucleic acid of (a) under stringent conditions. In one embodiment, at least one polymorphic position is selected from the group consisting of nucleotide position 1415 of a human SREBP1 cDNA sequence, nucleotide position 1904 of a human SREBP1 cDNA sequence and combinations thereof. In another embodiment, the nucleic acid comprises a sequence selected from the group consisting of SEQ ID NOs:3, 5 and 7 and complements thereof. In other embodiments, at least one polymorphic position resides in a coding position within the genomic sequence of the gene and/or the at least one polymorphic position residing in a coding position results in a missense mutation of the translated product of said gene. In yet a further embodiment, the invention comprises at least one polymorphic position and/or the 3′ end of a primer (e.g., a primer as disclosed herein) aligns with the at least one polymorphic position.

A vector comprising a nucleic acid of the present invention forms another aspect of the present invention. An isolated host cell transfected with such a vector forms another aspect of the present invention.

An isolated polypeptide comprising an amino acid sequence selected from the group consisting of SEQ ID NO:4, 6 and 8 forms another aspect of the present invention. A method of producing a polypeptide of the present invention forms another aspect of the present invention and, in one embodiment, the method comprises the step of culturing a host cell (such as those disclosed herein) under conditions in which the nucleic acid is expressed, whereby a polypeptide is produced.

Thus, in one embodiment, the present invention relates to a nucleic acid molecule comprising a single nucleotide polymorphism at a specific location in a nucleic acid sequence encoding a SREBP polypeptide (e.g., an SREBP1 polypeptide). In one particular embodiment, the present invention relates to a variant allele of a gene or a polynucleotide having a single nucleotide polymorphism, which variant allele differs from a reference allele by one nucleotide at the site(s) identified herein. Complements of these nucleic acid segments are also provided. The segments can be DNA or RNA, and can be double- or single-stranded. Segments can be, for example, 5, 10, 15, 20, 25, 30, 35, 40, 45, 50, 55, 60, 65, 70, 75, 80, 85, 90, 95, 100 or more bases long. In one embodiment, the specific location is selected from the group consisting of nucleotide position 1415 of a human SREBP1 cDNA sequence, nucleotide position 1904 of a human SREBP1 cDNA sequence and combinations thereof.

In another embodiment, the present invention relates to a reference or “wildtype” allele of a SREBP1 gene or polynucleotide having a single nucleotide polymorphism, which reference or wild type allele differs from a variant allele by one nucleotide at a site(s) identified herein. Complements of these nucleic acid segments are also provided. The segments can be DNA or RNA, and can be double- or single-stranded. Segments can be, for example, about 5-10, about 5-15, about 10-20, about 5-25, about 10-30, about 10-50 or about 10-100 bases long.

The present invention further provides variant and reference allele-specific oligonucleotides that hybridize to a nucleic acid molecule comprising a single nucleotide polymorphism or to the complement of the nucleic acid molecule. These oligonucleotides can be, for example, probes or primers (e.g., a sequence selected from SEQ ID NOs:9-19).

The present invention further provides oligonucleotides that can be used to amplify across a single nucleotide polymorphic site of the present invention. The present invention further provides oligonucleotides that can be used to sequence the amplified sequence (e.g., a sequence selected from SEQ ID NOs:9-19).

The present invention further provides a method of analyzing a nucleic acid from a DNA sample using amplification and sequencing primers of the present invention to assess whether a sample contains the reference or variant base (allele) at a polymorphic site (such as those identified herein). In one embodiment, the method comprises the steps of: (a) amplifying a sequence using appropriate PCR primers for amplifying across a polymorphic site, (b) sequencing the resulting amplified product using appropriate sequencing primers to sequence said product, and (c) determining whether the variant or reference base is present at the polymorphic site. Suitable primers are disclosed herein and can be selected, for example, from SEQ ID NOs:9-19)

The present invention further provides a method of analyzing a nucleic acid from DNA sample(s) from various populations using amplification and sequencing primers of the present invention to assess whether the sample(s) contain the reference or variant base (allele) at the polymorphic site, in an effort to identify populations having elevated plasma HDL levels and/or that are at a decreased risk for cardiovascular disease. In one embodiment, the method comprises the steps of: (a) amplifying a sequence using appropriate PCR primers for amplifying across a polymorphic site; (b) sequencing the resulting amplified product using appropriate sequencing primers to sequence said product; and (c) determining whether the variant or reference base is present at the polymorphic site. Optionally, a statistical analysis of a correlation between either the reference or variant allele at the polymorphic site(s) and the incidence of plasma HDL levels and/or cardiovascular disease can be determined. The polymorphism can be at a nucleotide position of SEQ ID NO:1 selected from the group consisting of 1415, 1904 and combinations thereof.

The present invention further provides oligonucleotides that can be used to genotype DNA sample(s) in order to assess whether the sample(s) contain the reference or variant base (allele) at a polymorphic site(s). The present invention also provides a method of using oligonucleotides that can be used to genotype a DNA sample to assess whether the sample contains the reference or variant base (allele) at the polymorphic site. In one embodiment, the method comprises the steps of amplifying a sequence using appropriate PCR primers for amplifying across a polymorphic site, subjecting the product of said amplification to a genetic bit analysis (GBA) reaction, and analyzing the result.

The present invention also provides a method of using oligonucleotides that can be used to genotype DNA sample(s) to identify a population (e.g., an ethnic population) that might be at a decreased risk of developing a cardiovascular disease and/or a have elevated plasma HDL levels. In one embodiment, the method comprises the steps of: (a) amplifying a sequence using appropriate PCR primers for amplifying across a polymorphic site; (b) subjecting the product of said amplification to a genetic bit analysis (GBA) reaction, and (c) analyzing the result. The method can optionally comprise the step of determining a statistical association between either the reference or variant allele at the polymorphic site(s) to the incidence of cardiovascular disease and/or HDL-related disease.

The present invention further provides a method of analyzing a nucleic acid derived from an individual. The method allows a determination of whether a reference or variant base is present at any one or more of the polymorphic sites disclosed herein. Optionally, a set of bases occupying a set of the polymorphic sites shown herein is determined. This type of analysis can be performed on a number of individual(s), who are also tested (previously, concurrently or subsequently) for the presence of a disease phenotype. The presence or absence of disease phenotype is then correlated with a base or set of bases present at the polymorphic site or sites in the individual(s) tested. In an embodiment of the method, the one or more polymorphic sites is selected from the group consisting of nucleotide position 1415 of SEQ ID NO:1, nucleotide position 1904 of SEQ ID NO:1 and combinations thereof.

Thus, the present invention further relates to a method of predicting the presence, absence, likelihood of the presence or absence, or severity of a particular phenotype or disorder (e.g. elevated plasma HDL levels and/or cardiovascular disease) associated with a particular genotype (e.g., SEQ ID NOs:1, 3, 5 and 7). In one embodiment, the method comprises the steps of: (a) obtaining a nucleic acid sample from an individual; and (b) determining the identity of one or more bases (nucleotides) at one or more specific (e.g., polymorphic) sites of nucleic acid molecules described herein, wherein the presence of a particular base at that site is correlated with a specified phenotype or disorder, thereby predicting the presence, absence, likelihood of the presence: or absence, or severity of the phenotype or disorder in the individual, wherein the phenotype or disorder is, for example, elevated plasma HDL levels and/or cardiovascular disease, or a decreased risk of cardiovascular disease. In an embodiment of the method, the one or more specific sites is selected from the group consisting of nucleotide position 1415 of a human SREBP1 cDNA sequence (e.g., SEQ ID NO:1), nucleotide position 1904 of a human SREBP1 cDNA sequence (e.g., SEQ ID NO:1) and combinations thereof.

The present invention further relates to polynucleotides having one or more variant alleles. The present invention also relates to such polynucleotides lacking a start codon. The invention further relates to polynucleotides of the present invention comprising one or more variant alleles wherein the polynucleotides encode a polypeptide of the present invention. The present invention also relates to polypeptides of the present invention comprising one or more variant amino acids encoded by one or more variant alleles.

In another aspect, the present invention further relates to antisense oligonucleotides corresponding to the polynucleotides of the present invention. In one embodiment, such antisense oligonucleotides are adapted to discriminate against a reference or variant allele of the polynucleotide, for example at one or more polymorphic sites of said polynucleotide. In one embodiment, the one or more polymorphic positions is selected from the group consisting of nucleotide position 1415 of a human SREBP1 cDNA sequence, nucleotide position 1904 of a human SREBP1 cDNA sequence (e.g., SEQ ID NO:1) and combinations thereof.

The present invention also relates to antibodies directed against a polypeptide of the present invention (e.g., a polypeptide shown in SEQ ID NOs: 4, 6 and/or 8). In one embodiment, such antibodies are adapted to discriminate against the reference (i.e., wildtype) or variant allele of the polypeptide, for example at one or more polymorphic sites of said polynucleotide. In one embodiment, the one or more polymorphic positions is selected from the group consisting of nucleotide position 1415 of a human SREBP1 cDNA sequence, nucleotide position 1904 of a human SREBP1 cDNA sequence (e.g., SEQ ID NO:1) and combinations thereof.

The present invention additionally relates to a recombinant vector comprising an isolated nucleic acid molecule of the present invention, and to a host cell comprising the recombinant vector. Methods of making such vectors and host cells are also described, in addition to uses of the vector in the production, via recombinant techniques, of the polypeptides and/or peptides provided herein. Synthetic methods for producing the polypeptides and polynucleotides of the present invention are also provided. Further, diagnostic methods are also described for detecting diseases, disorders, and/or conditions related to the polypeptides and polynucleotides provided herein, as well beneficial attributes of the polypeptides and polynucleotides of the present invention, such as elevated plasma HDL levels and/or a decreased risk of cardiovascular disease. Also disclosed are therapeutic methods for treating such diseases, disorders, and/or conditions as well as methods of employing the polypeptides and polynucleotides of the present invention to benefit individuals having decreased plasma HDL levels and/or who are at risk for cardiovascular disease. The present invention also relates to screening methods for identifying binding partners of the polypeptides.

The present invention further provides an isolated polypeptide having an amino acid sequence encoded by a polynucleotide described herein (e.g., SEQ ID NOs: 3, 5, and/or 7 and complements thereof).

The present invention additionally relates to the identification of SNPs that have been determined to represent a random sampling of SNPs throughout the genome of a DNA sample, or sample(s), such as the SNPs provided herein (e.g., a variation from wildtype at nucleotide position 1415 of a human SREBP1 cDNA sequence, a variation from wildtype at nucleotide position 1904 of a human SREBP1 cDNA sequence (e.g., SEQ ID NO:1) and combinations thereof).

The present invention additionally relates to the use of such randomly distributed SNPs as a technique of increasing the accuracy of ethnic assignments for genomic control DNA sample(s) of the present invention. The increased ethnic accuracy of such genomic controls results in an increased statistical confidence in the phenotype association data obtained for any one or more SNPs of the present invention.

The present invention further relates to the use of such genomic control SNPs for clustering individuals to confirm known gene pool/racial/ethnic groups or to reveal cryptic SNPs in a DNA sample(s).

The present invention additionally relates to a method of analyzing at least one nucleic acid sample, comprising the steps of: (a) obtaining a nucleic acid sample from one or more individuals; and (b) determining the nucleic acid sequence at one or more polymorphic positions in a gene encoding a SREBP1 protein. In an embodiment of the method, the one or more polymorphic positions is selected from the group consisting of nucleotide position 1415 of a human SREBP1 cDNA sequence (e.g., SEQ ID NO:1), nucleotide position 1904 of a human SREBP1 cDNA sequence (e.g., SEQ ID NO:1) and combinations thereof.

The present invention further relates to a method of analyzing at least one nucleic acid sample, further comprising the steps of: (a) testing each individual for the presence of a disease phenotype; and (b) correlating the presence of the disease phenotype with the sequence at the one or more polymorphic positions. In one embodiment, the disease phenotype comprises a cardiovascular disease. In another embodiment of the method, the one or more polymorphic positions is selected from the group consisting of nucleotide position 1415 of SEQ ID NO:1, nucleotide position 1904 of SEQ ID NO:1 and combinations thereof.

The present invention further relates to a method of analyzing at least one nucleic acid sample, wherein the one or more polymorphic positions of said nucleic acid sequence is a polymorphic position specified herein for a SREBP1 gene. In an embodiment of the method, the one or more polymorphic positions is selected from the group consisting of nucleotide position 1415 of SEQ ID NO:1, nucleotide position 1904 of SEQ ID NO:1 and combinations thereof.

The present invention further relates to a method of constructing haplotypes using the isolated nucleic acids disclosed in the Figures and/or referred to in the Sequence Listing and/or described herein, comprising the step of grouping at least two of the nucleic acids. The present invention also relates to a method of constructing the haplotypes further comprising the step of using the haplotypes to identify an individual for the presence of a given phenotype, and correlating the presence of the phenotype with the haplotype. In one example, the phenotype comprises a decreased risk for cardiovascular disease. In another example, the phenotype comprises elevated plasma HDL levels.

The present invention further relates to a method of identifying an individual having a decreased risk of developing a cardiovascular disorder and/or elevated HDL levels. In one embodiment, the method comprises the steps of: (a) obtaining a nucleic acid sample from an individual; (b) amplifying one or more sequences from the sample using appropriate PCR primers for amplifying across at least one polymorphic position; (c) comparing the at least one polymorphic position with a known data set; and (d) determining whether the result correlates with a given phenotype, such as a decreased risk for developing a disorder, for example a cardiovascular disorder, or elevated plasma HDL levels. In one embodiment, the appropriate PCR primers are selected from the group consisting of SEQ ID NOs:9-19.

The present invention further relates to a library of nucleic acids, each of which comprises one or more polymorphic positions within a gene encoding a human SREBP1 protein, wherein the polymorphic positions are selected from the polymorphic positions provided herein (e.g., nucleotide position 1415 of a human SREBP1 cDNA sequence (e.g., SEQ ID NO:1), nucleotide position 1904 of a human SREBP1 cDNA sequence (e.g., SEQ ID NO:1) and combinations thereof). The present invention further relates to a library of nucleic acids, wherein the sequence at the polymorphic position is selected from the sequences provided herein. The present invention also relates to a library of nucleic acids, wherein the library of isolated sequences represents the complimentary sequence of the nucleotide sequences described herein.

The present invention further relates to a kit for identifying a subject that has a decreased risk of developing a cardiovascular disease and/or has elevated plasma HDL levels. In one embodiment, the kit comprises: (a) one or more sequencing primers; and (b) one or more sequencing reagents, wherein the sequencing primers are primers that hybridize to at least one polymorphic position in a human SREBP1 gene. In another embodiment of the kit, the at least one polymorphic position is selected from the group consisting of nucleotide position 1415 of a human SREBP1 cDNA sequence, nucleotide position 1904 of a human SREBP1 cDNA sequence (e.g. SEQ ID NO:1) and combinations thereof. In yet another embodiment of the kit, the one or more sequencing primers comprise a nucleotide sequence selected from the group consisting of SEQ ID NOs:9-19, complements thereof and combinations thereof. In yet another embodiment of the kit, the one or more sequencing primers is labeled. A kit of the present invention can further comprise instructions for use in diagnosing a subject as having, or having a predisposition for developing, a cardiovascular disease or as having, or having a predisposition for developing, elevated plasma HDL levels, for example plasma HDL levels greater than about 35-40 mg/dL.

The present invention additionally relates to a method for predicting the likelihood that an individual will be diagnosed as being at decreased risk of developing a cardiovascular disorder and/or having elevated plasma HDL levels. In one embodiment, the method comprises the steps of: (a) obtaining a nucleic acid sample from an individual to be assessed; and (b) determining the nucleotide present at one or more polymorphic position(s) of a SREBP1 gene, wherein the presence of the reference nucleotide at the one or more polymorphic position(s) indicates that the individual has a lower likelihood of being diagnosed as being at risk of developing a cardiovascular disorder as compared to an individual having an alternate allele at the polymorphic position(s). In an embodiment of the method, the one or more polymorphic positions is selected from the group consisting of nucleotide position 1415 of SEQ ID NO:1, nucleotide position 1904 of SEQ ID NO:1 and combinations thereof.

The invention further relates to a method for predicting the likelihood that an individual will be diagnosed as being at risk of developing a cardiovascular disorder. In one embodiment, the method comprises the steps of: (a) obtaining a DNA sample from an individual to be assessed; and (b) determining the nucleotide present at a nucleotide position selected from the group consisting of nucleotide 1415 of SEQ ID NO:1, nucleotide position 1904 of SEQ ID NO:1 and combinations thereof, wherein the presence of an “A” (adenine) at a nucleotide position selected from the group consisting of 1415 of SEQ ID NO:1, 1904 of SEQ ID NO:1 and combinations thereof indicates that the individual has a lower likelihood of being diagnosed with a cardiovascular disorder than an individual having a “G” (guanine) at a nucleotide position selected from the group consisting of 1415 of SEQ ID NO:1, 1904 of SEQ ID NO:1 and combinations thereof. In one embodiment of the method, the determining comprises: (a) contacting the nucleic acid sample with a nucleic acid probe or primer selected from the group consisting of SEQ ID NO:9-19 to form a hybridized structure; and (b) detecting the presence of the hybridized structure.

In yet another embodiment of the method, the probe or primer is capable of specifically hybridizing to a nucleic acid sequence comprising a nucleotide selected from the group consisting of nucleotide 1415 of SEQ ID NO:1, nucleotide 1904 of SEQ ID NO:1 and combinations thereof. In another embodiment of the method, the nucleic acid probe or primer is capable of specifically hybridizing to a nucleic acid sample comprising an adenine at a position selected from the group consisting of nucleotide 1415 of a human SREBP1 cDNA sequence (e.g., SEQ ID NO:1), nucleotide 1904 of a human SREBP1 cDNA sequence (e.g., SEQ ID NO:1) and combinations thereof.

The present invention further relates to a method for genotyping an individual. In one embodiment, the method comprises the steps of: (a) obtaining a nucleic acid sample from the individual; (b) determining a nucleotide present at least one polymorphic position, and (c) comparing said at least one polymorphic position with a known data set, for example a reference nucleotide sequence, such as a reference allele. In one embodiment of the method, the at least one polymorphic position is selected from the group consisting of nucleotide position 1415 of SEQ ID NO:1, nucleotide position 1904 of SEQ ID NO:1 and combinations thereof.

Therefore, it is an object of the present invention to provide a method of predicting the likelihood that a subject will be diagnosed with a cardiovascular disorder and/or exhibit an elevated plasma HDL level. This and other objects are achieved by employing a polynucleotide and/or polypeptide sequence of the present invention, as disclosed herein.

An object of the present invention having been stated, other objects, as well as other advantages, will become evident as the description proceeds in connection with the Figures and the Examples below.

### BRIEF DESCRIPTION OF THE DRAWINGS

FIGS. 1A-1B depict the nucleotide sequence of a SREBP1 gene comprising guanine (“G”) nucleotides at polymorphic positions 1415 and 1904.

FIGS. 2A-2F depict the amino acid sequence of a SREBP1 polypeptide comprising valine residues at positions 417 and 580.

FIGS. 3A-3B depict the nucleotide sequence of a SREBP1 gene comprising an adenine (“A”) nucleotide at polymorphic position 1415 and 1904.

FIGS. 4A-4F depict the amino acid sequence of a SREBP1 polypeptide comprising a methionine residue at position 417 and a valine residue at position 580.

FIGS. 5A-5B depict the nucleotide sequence of a SREBP1 gene comprising a guanine (“G”) nucleotide at polymorphic position 1415 and an adenine (“A”) nucleotide at polymorphic position 1904

FIGS. 6A-6F depict the amino acid sequence of a SREBP1 polypeptide comprising a valine residue at position 417 and a methionine residue at position 580.

FIGS. 7A-7B depict the nucleotide sequence of a SREBP1 gene comprising an adenine (“A”) nucleotide at polymorphic position 1415 and an adenine (“A”) nucleotide at polymorphic position 1904.

FIGS. 8A-8F depict the amino acid sequence of a SREBP1 polypeptide comprising a methionine residue at position 417 and a methionine at position 580.

### BRIEF DESCRIPTION OF THE SEQUENCES IN THE SEQUENCE LISTING

SEQ ID NO:1 is a nucleic acid sequence of a human SREBP1 gene comprising a guanine (“G”) nucleotide at positions 1415 and 1904 and encoding a SREBP1 polypeptide comprising valine residues at positions 417 and 580, respectively, of the SREBP1 polypeptide sequence.

SEQ ID NO:2 is a polypeptide sequence of a human SREBP1 polypeptide comprising valine residues at positions 417 and 580 of the SREBP1 polypeptide sequence.

SEQ ID NO:3 is a nucleic acid sequence of a human SREBP1 gene comprising an adenine (“A”) nucleotide at position 1415 and a guanine (“G”) nucleotide at position 1904 and encoding a SREBP1 polypeptide comprising a methionine residue at position 417 and a valine residue at position 580 of the SREBP1 polypeptide sequence.

SEQ ID NO:4 is a polypeptide sequence of a human SREBP1 polypeptide comprising a methionine residue at position 417 and a valine residue at position 580 of the SREBP1 polypeptide sequence.

SEQ ID NO:5 is a nucleic acid sequence of a human SREBP1 gene comprising an a guanine (“G”) nucleotide at position 1415 and an adenine (“A”) nucleotide at position 1904 and encoding a SREBP1 polypeptide comprising a valine residue at position 417 and a methionine residue at position 580 of the SREBP1 polypeptide sequence.

SEQ ID NO:6 is a polypeptide sequence of a human SREBP1 polypeptide comprising a valine residue at position 417 and a methionine residue at position 580 of the SREBP1 polypeptide sequence.

SEQ ID NO:7 is a predicted nucleic acid sequence of a human SREBP1 gene comprising an adenine (“A”) nucleotide at positions 1415 and 1904 and encoding a SREBP1 polypeptide comprising methionine residues at positions 417 and 580 of the SREBP1 polypeptide sequence.

SEQ ID NO:8 is a polypeptide sequence of a human SREBP1 polypeptide comprising a methionine residue at positions 417 and 580 of the SREBP1 polypeptide sequence.

SEQ ID NO:9 is a nucleic acid sequence of a first probe for a M417V SREBP1 variant.

SEQ ID NO:10 is a nucleic acid sequence of a second probe for a M417V SREBP1 variant.

SEQ ID NO:11 is a nucleic acid sequence of a first primer for a M417V SREBP1 variant.

SEQ ID NO:12 is a nucleic acid sequence of a second primer for a M417V SREBP1 variant.

SEQ ID NO:13 is a nucleic acid sequence of a first probe for a M580V SREBP1 variant.

SEQ ID NO:14 is a nucleic acid sequence of a second probe for a M580V SREBP1 variant.

SEQ ID NO:15 is a nucleic acid sequence of a first primer for a M580V SREBP1 variant.

SEQ ID NO:16 is a nucleic acid sequence of a second primer for a M580V SREBP1 variant.

SEQ ID NO:17 is a nucleic acid comprising a M13 reverse sequencing primer.

SEQ ID NO:18 is a nucleic acid comprising a M13 forward sequencing primer.

SEQ ID NO:19 is a nucleic acid comprising a flanking primer.

### DETAILED DESCRIPTION OF THE INVENTION

The present invention relates to nucleic acid molecules that comprise a single nucleotide polymorphism (SNP) at one or more specific locations. The nucleic acid molecules, (e.g., genes) that comprise the SNP have two alleles, referred to herein as a reference allele and a variant allele. The reference allele (also referred to as a “prototypical” or “wildtype” allele) has been designated arbitrarily and typically corresponds to the nucleotide sequence of the native form of the nucleic acid molecule. The variant allele differs from the reference allele by at least one nucleotide at the site(s) identified herein (e.g., position 1415 and/or 1904 of the wildtype SREBP1 polypeptide sequence shown in SEQ ID NO:1). The present invention also relates to variant alleles of the described genes and to complements of the variant alleles. The present invention further relates to portions of the variant alleles and portions of complements of the variant alleles which comprise (encompass) the site of the SNP and can be of variable length. Portions can be, for example, 5-10, 5-15, 10-20, 5-25, 10-30, 10-50 or 10-100 bases long. For example, a portion of a variant allele can comprise a single nucleotide polymorphism (the nucleotide differs from the reference allele at that site) and a variable number of additional nucleotides that flank the site in the variant allele (e.g., twenty nucleotides). These additional nucleotides can be on one or both sides of the polymorphism. Polymorphisms, which are a subject of the present invention, are defined herein.

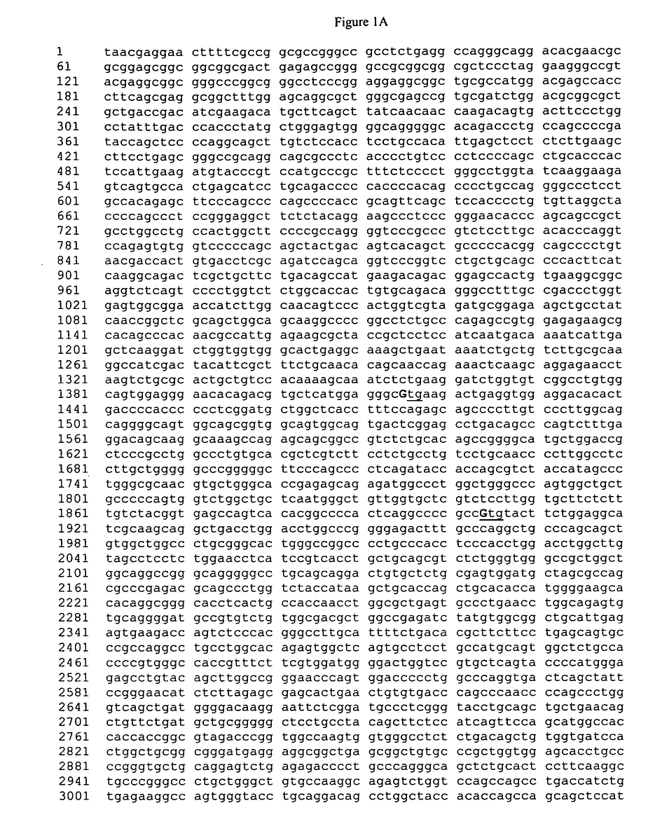

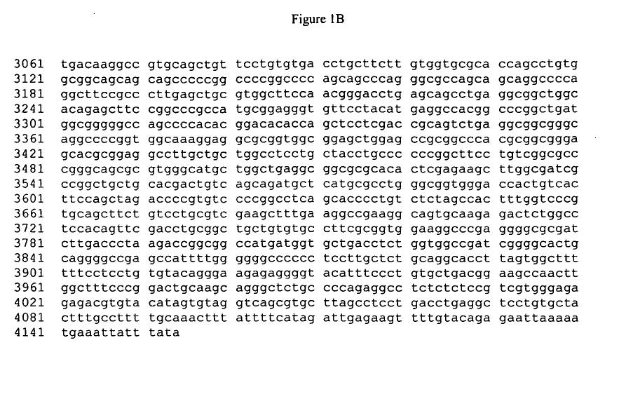

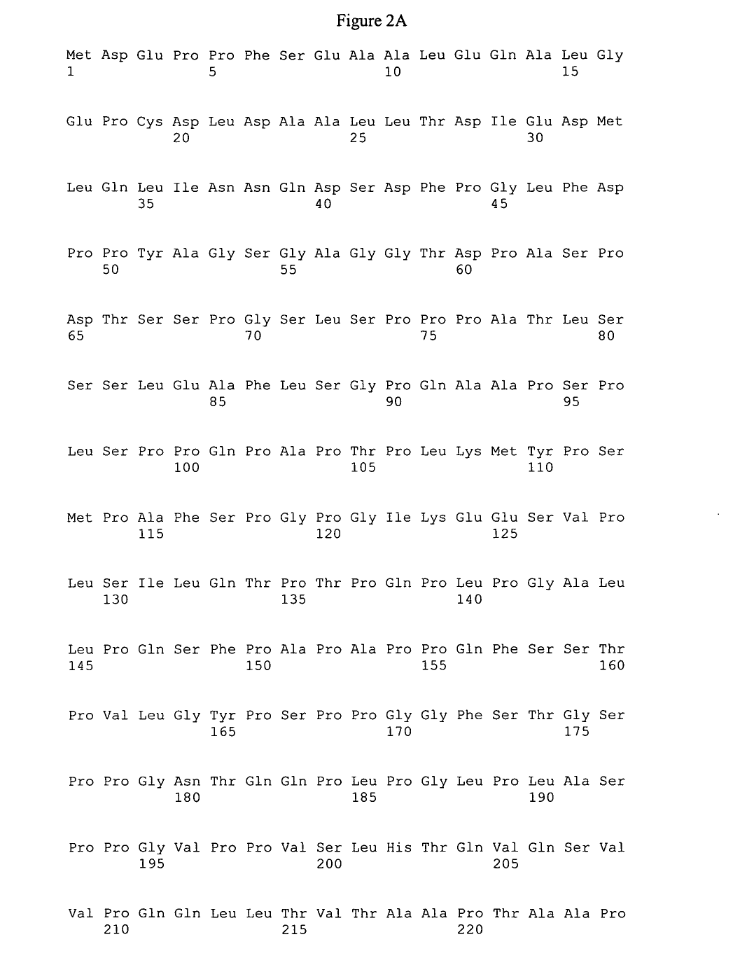

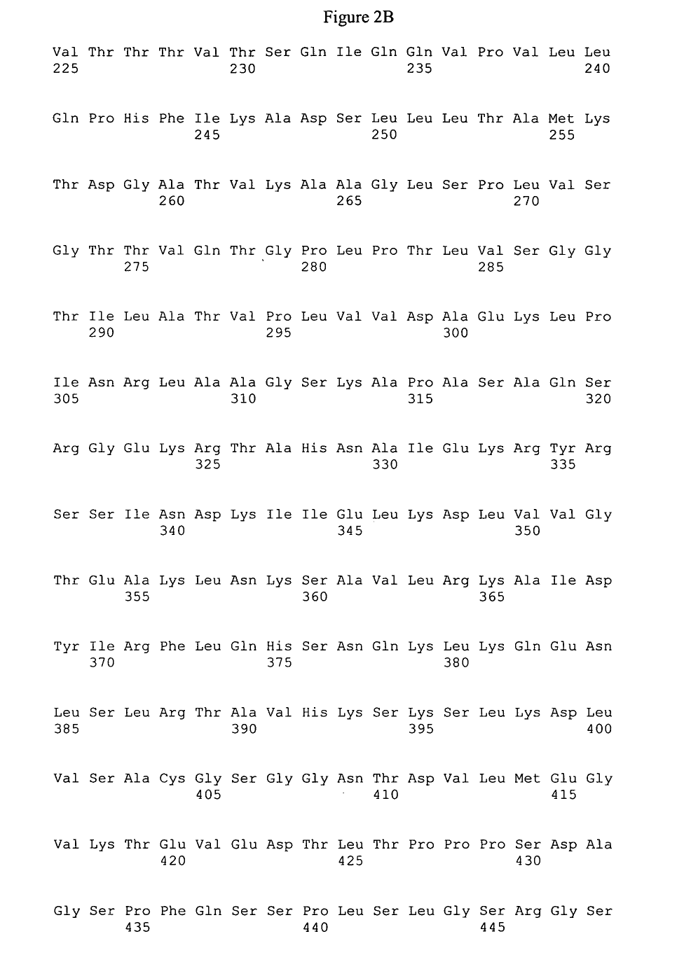

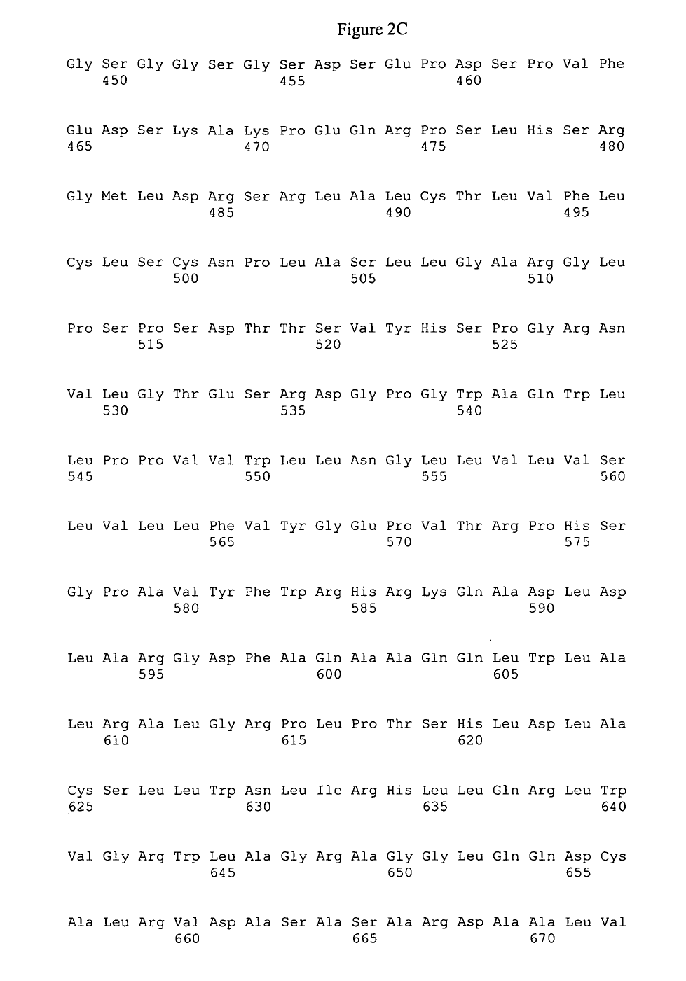

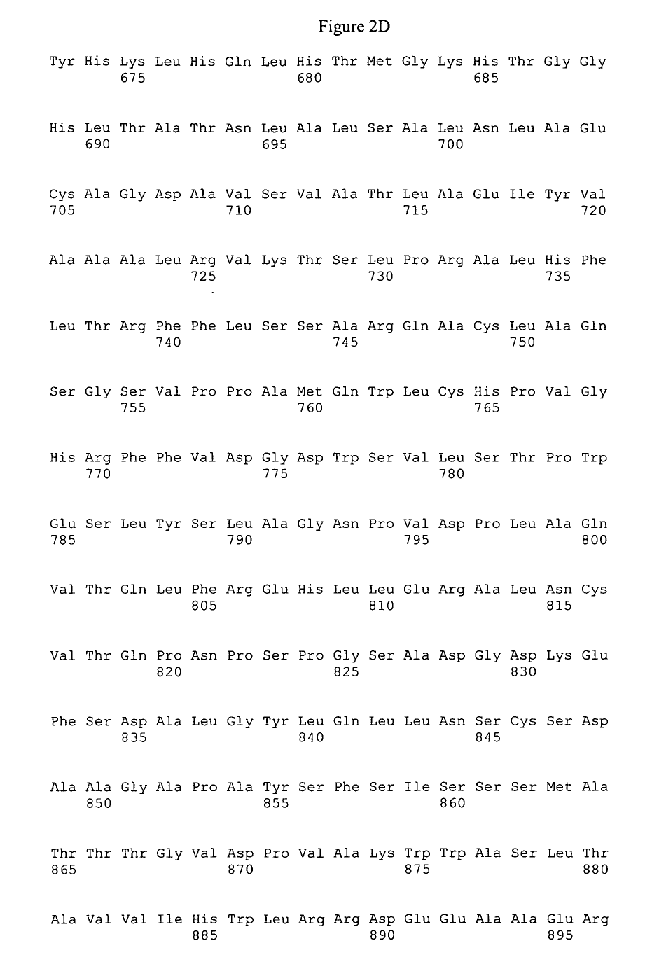

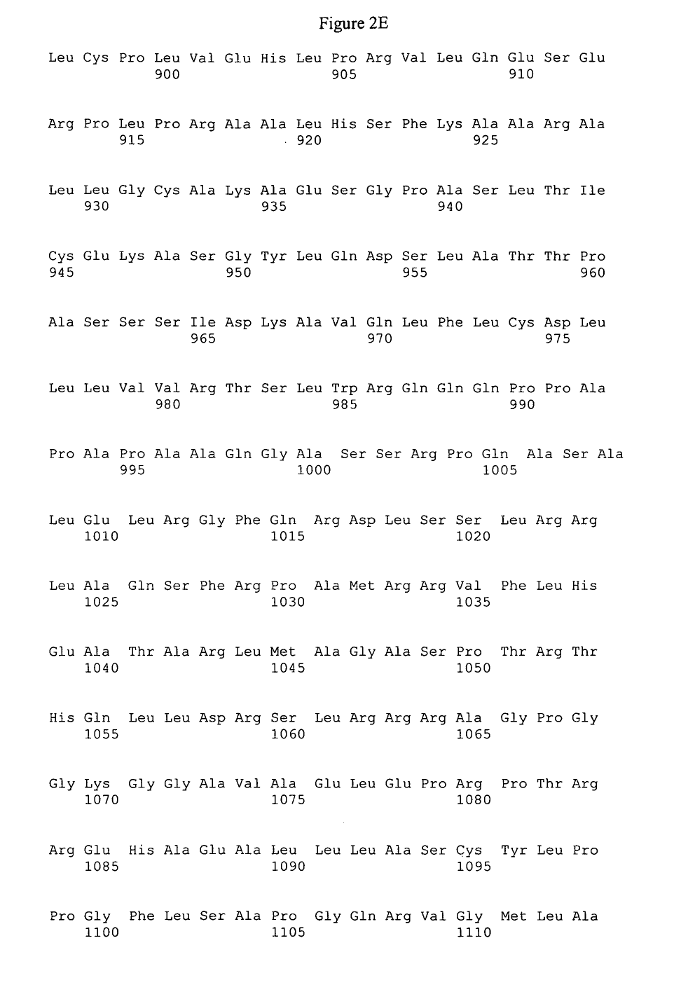

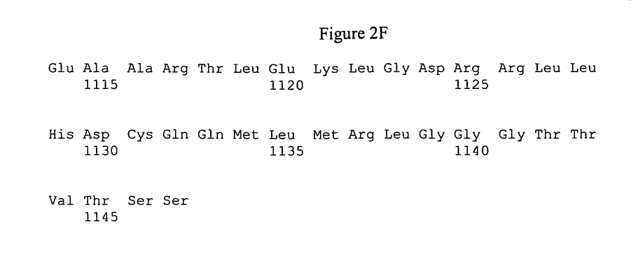

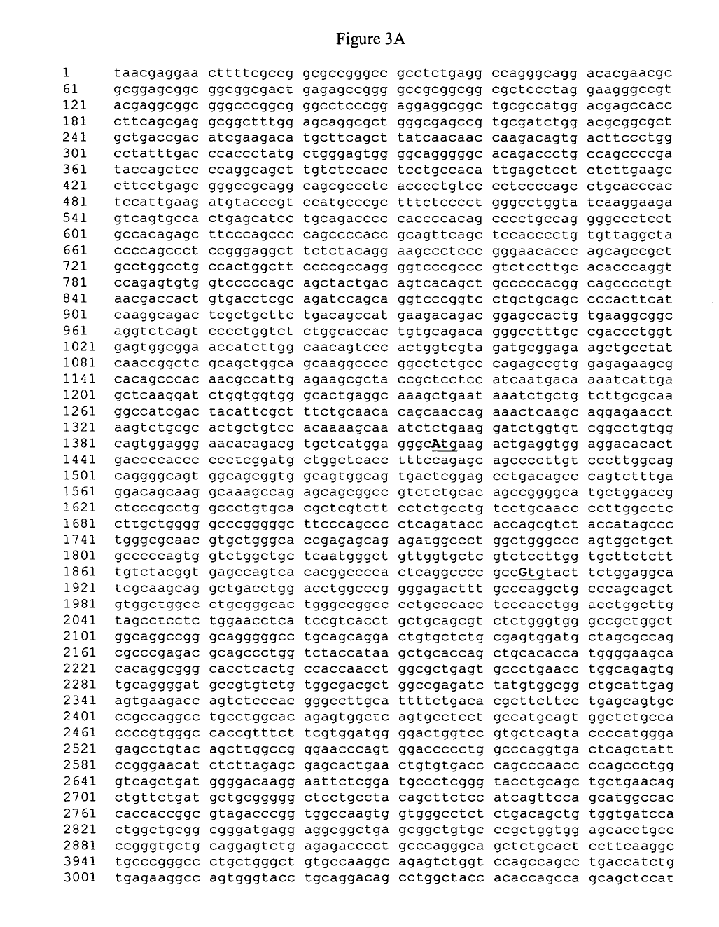

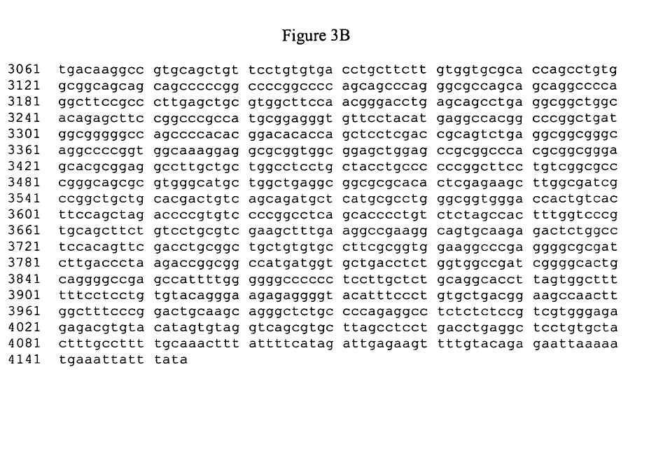

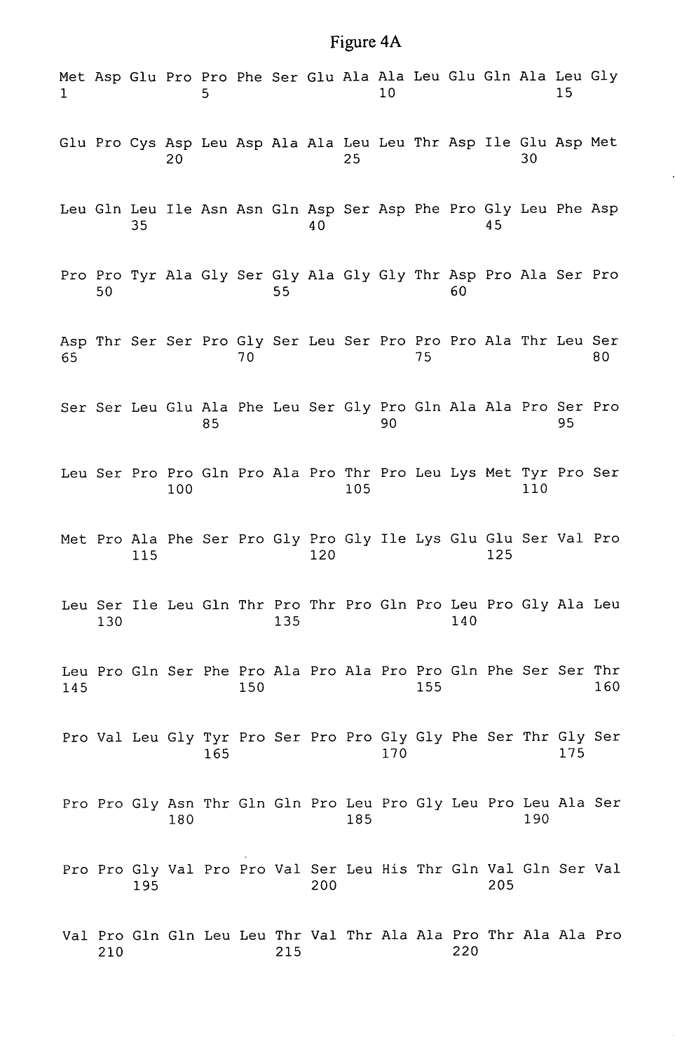

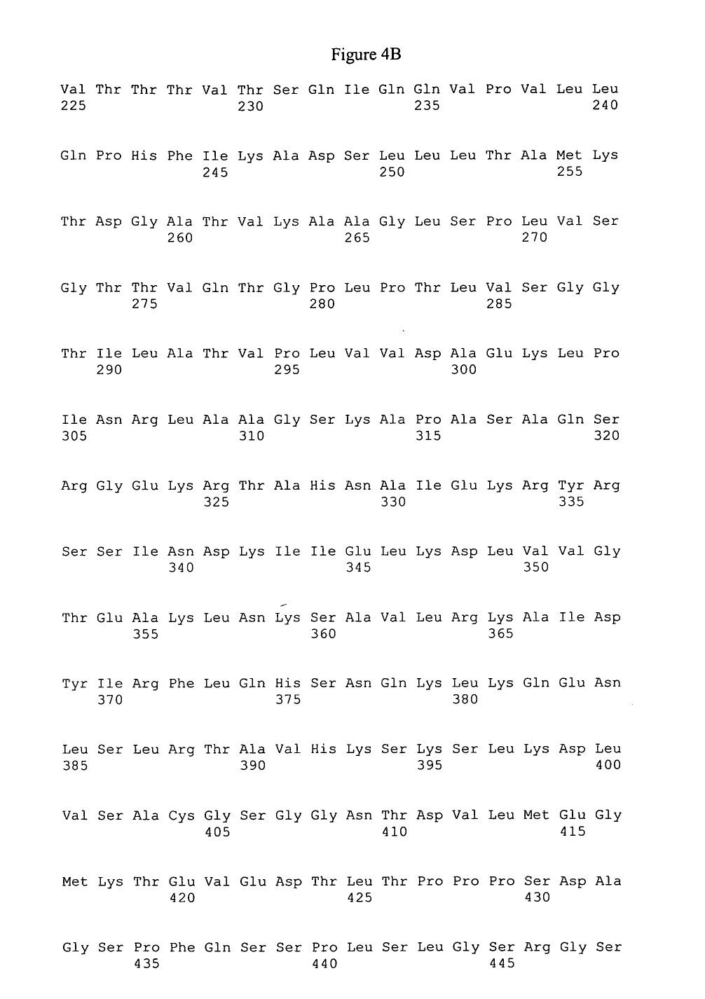

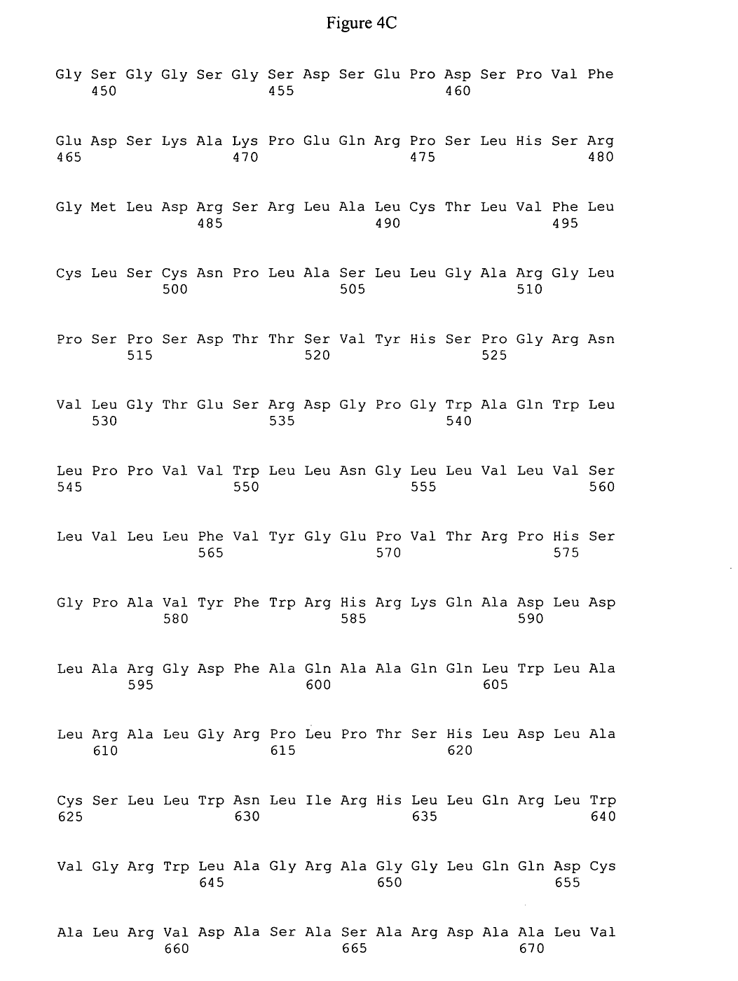

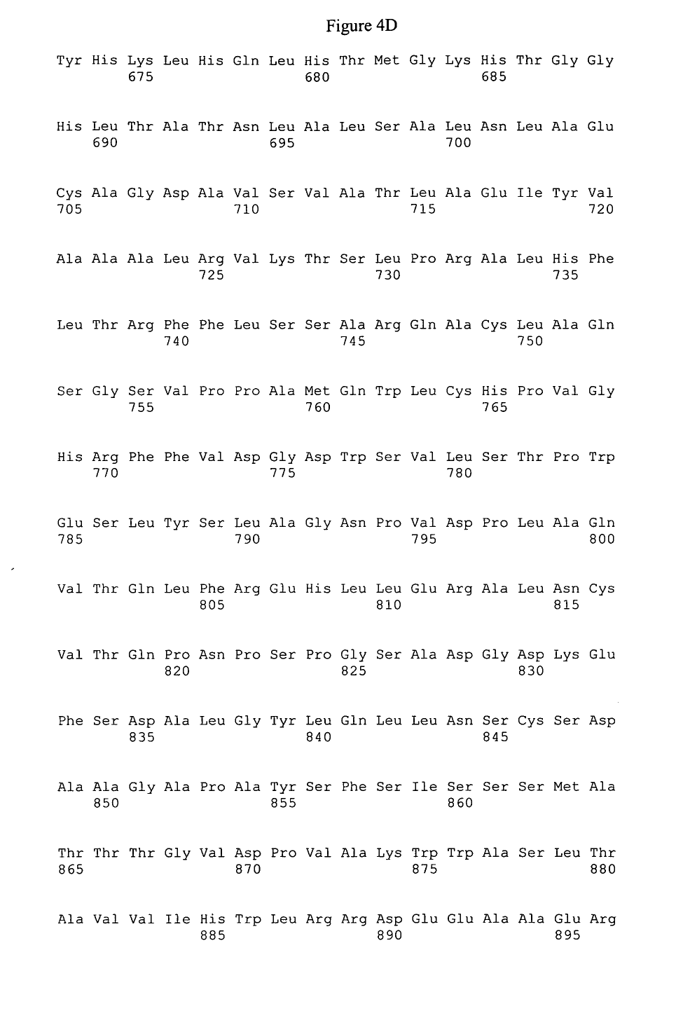

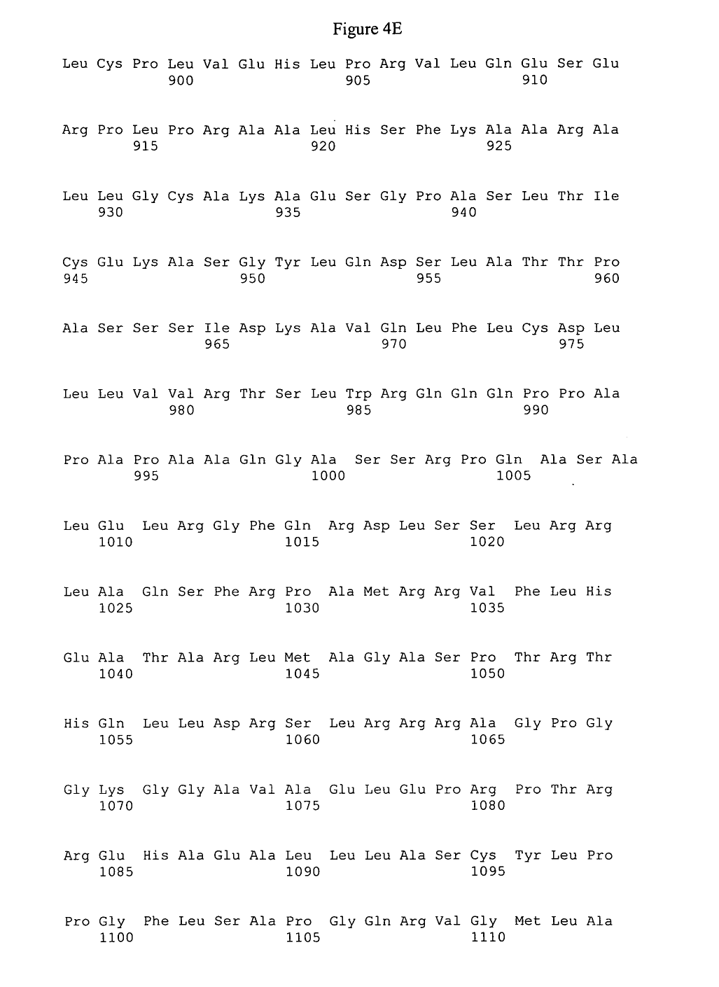

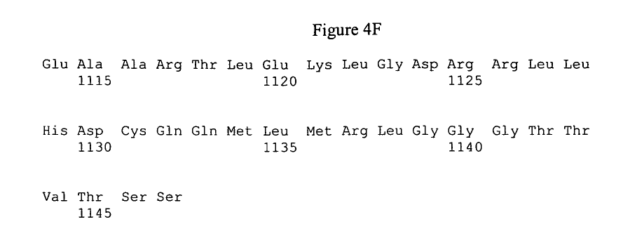

For example, the present invention relates to a portion of a human SREBP1 gene (FIGS. 1A-1B SEQ ID NO:1) having a nucleotide sequence according to FIGS. 3A-3B (SEQ ID NO:3), comprising a single nucleotide polymorphism at a specific position (e.g., nucleotide 1415 and/or 1904). The reference nucleotide for this polymorphic form of SREBP1 is shown in FIGS. 1A-1B (SEQ ID NO:1) and the variant nucleotide is shown in FIGS. 3A-3B (SEQ ID NO:3). The variant nucleotide encodes the polypeptide shown in FIGS. 4A-4F (SEQ ID NO:4). In one embodiment, a nucleic acid molecule of the present invention comprises the variant (alternate) nucleotide at the polymorphic position.

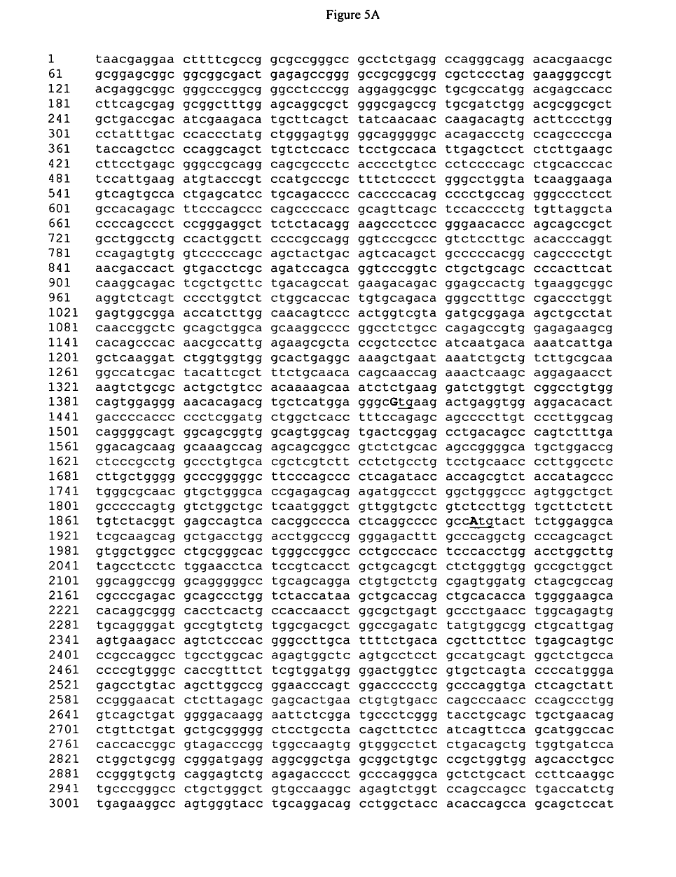

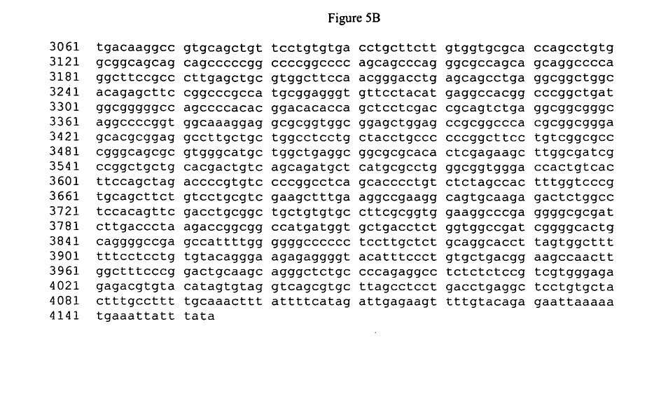

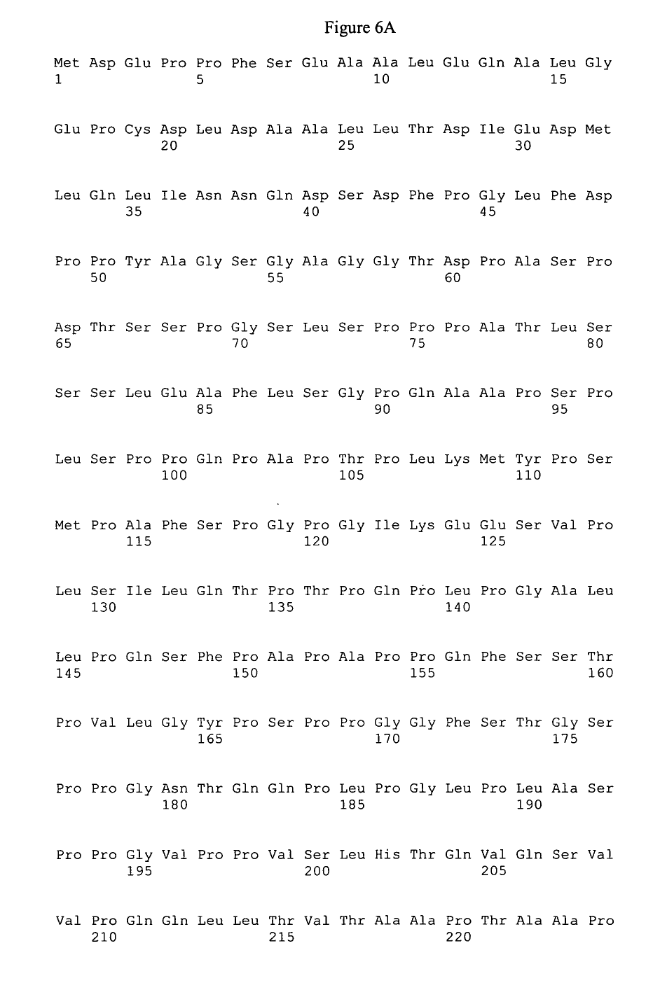

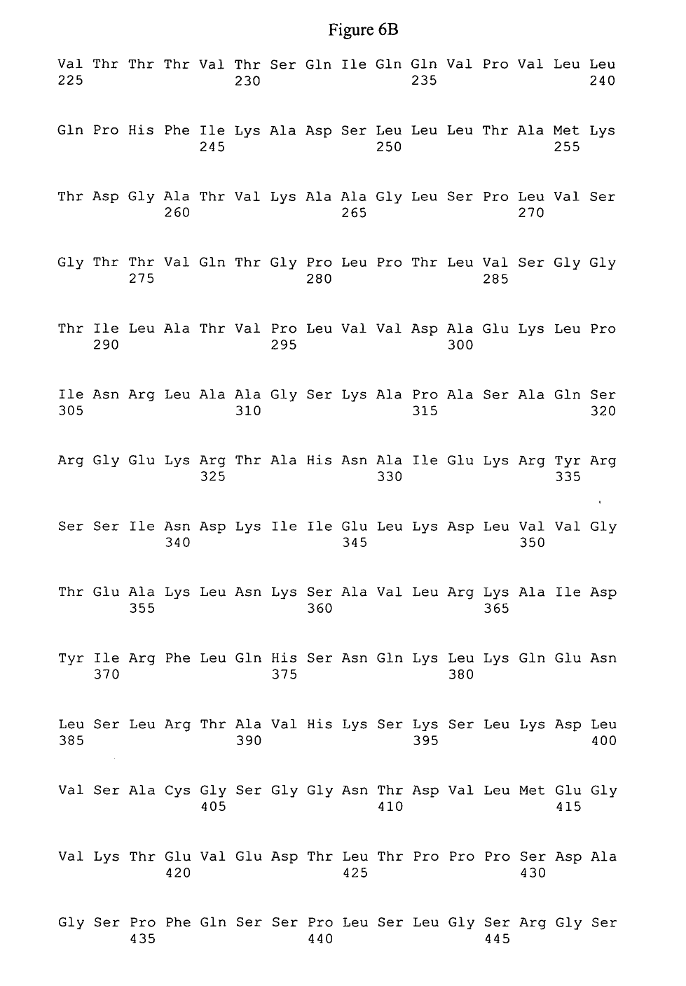

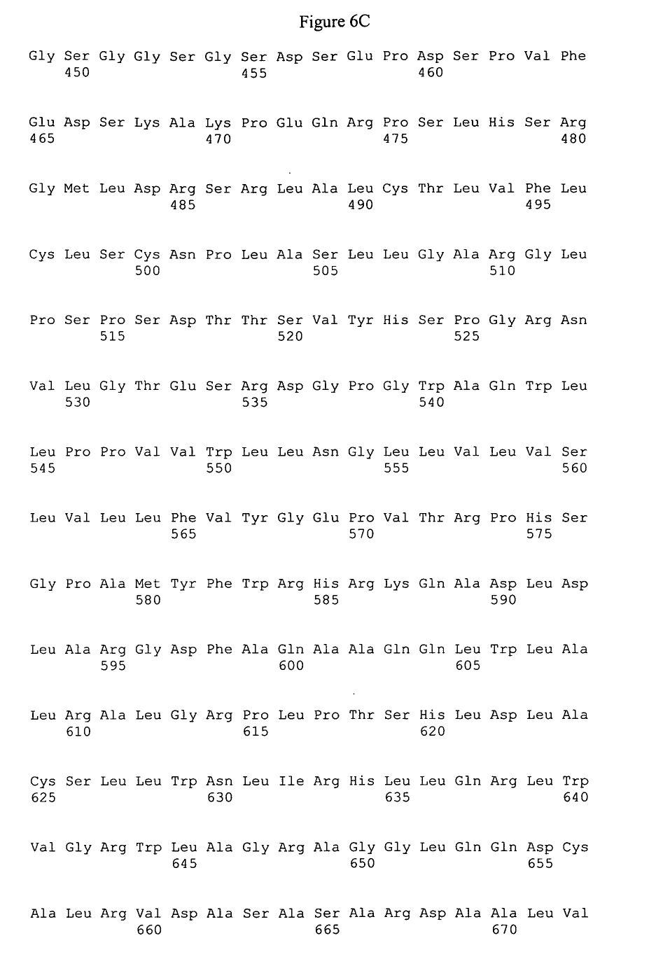

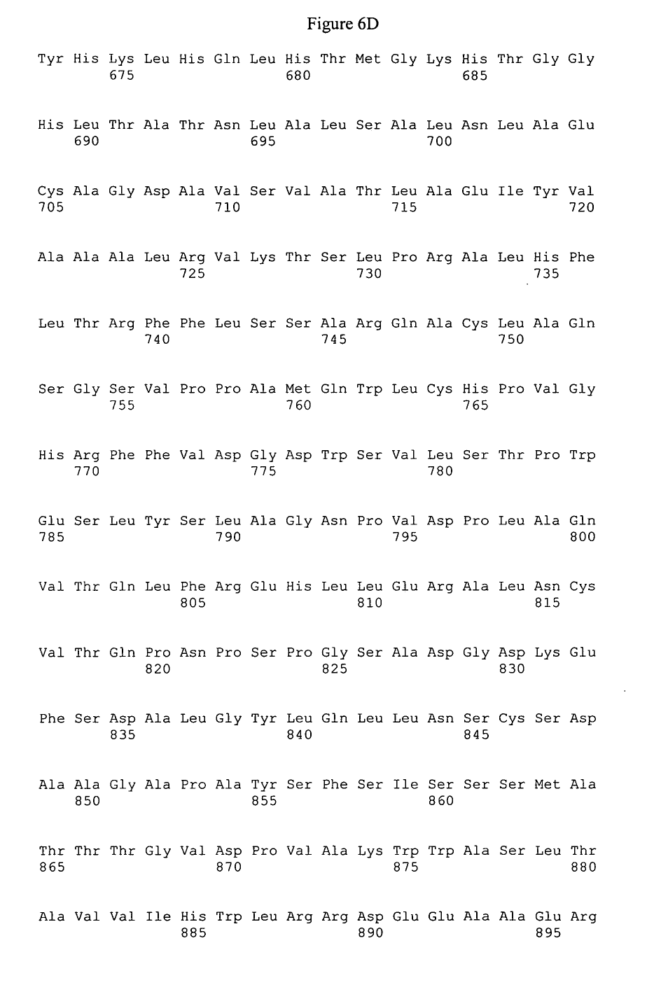

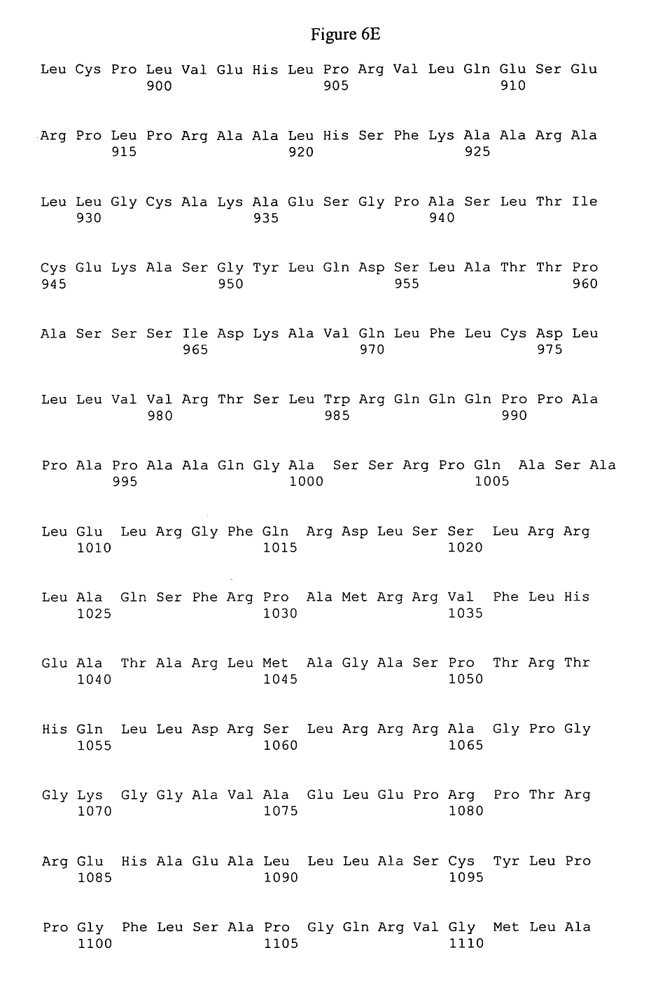

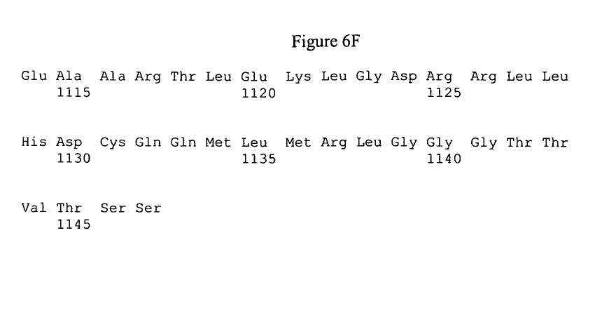

In another example, the present invention relates to a portion of a human SREBP1 gene (FIGS. 1A-1B; SEQ ID NO:1) having a nucleotide sequence according to FIGS. 5A-5B (SEQ ID NO:5) comprising a single nucleotide polymorphism at a specific position (e.g., nucleotide 1904). The reference nucleotide for this polymorphic form of SREBP1 is shown in FIGS. 1A-1B (SEQ ID NO:1) and the variant nucleotide is shown in FIG. 5A-5B (SEQ ID NO:5). The variant nucleotide encodes the polypeptide shown in FIGS. 6A-6F (SEQ ID NO:6). In one embodiment, a nucleic acid molecule of the present invention comprises the variant (alternate) nucleotide at the polymorphic position.

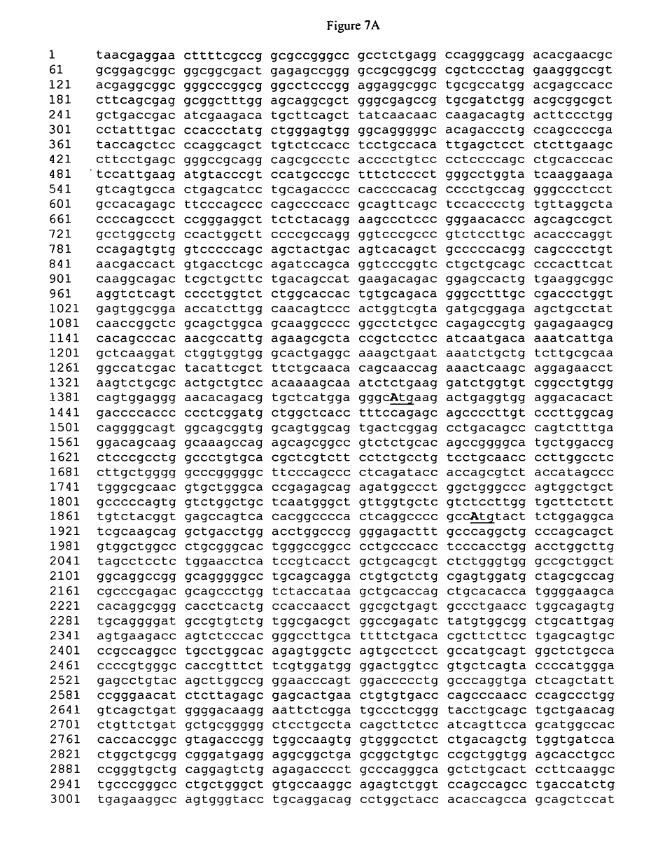

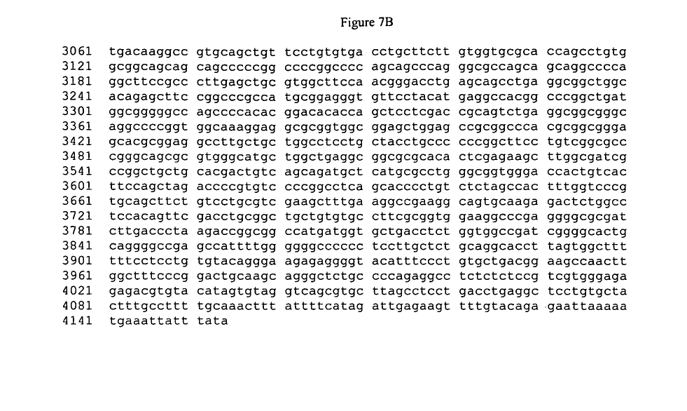

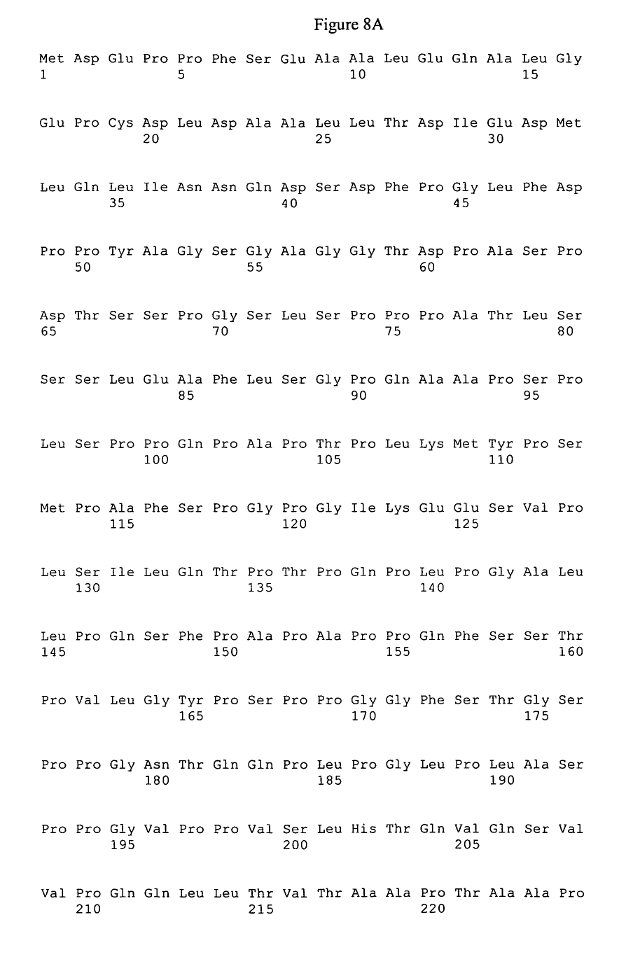

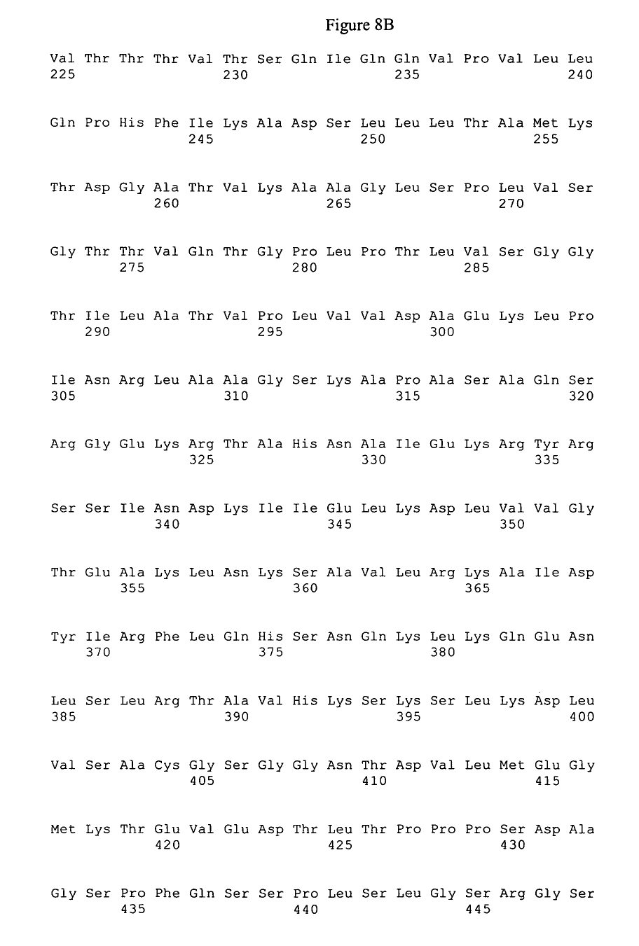

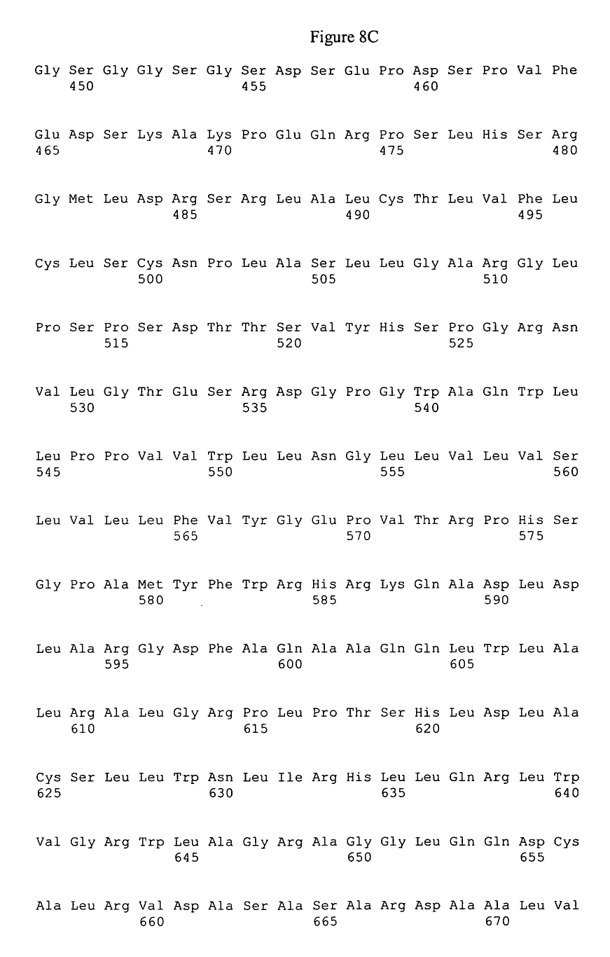

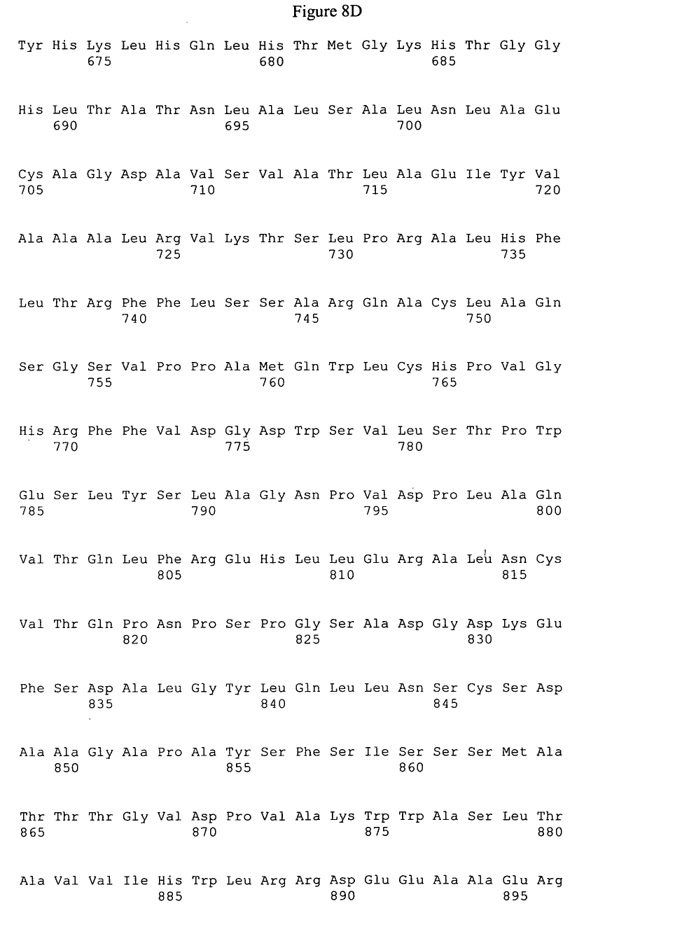

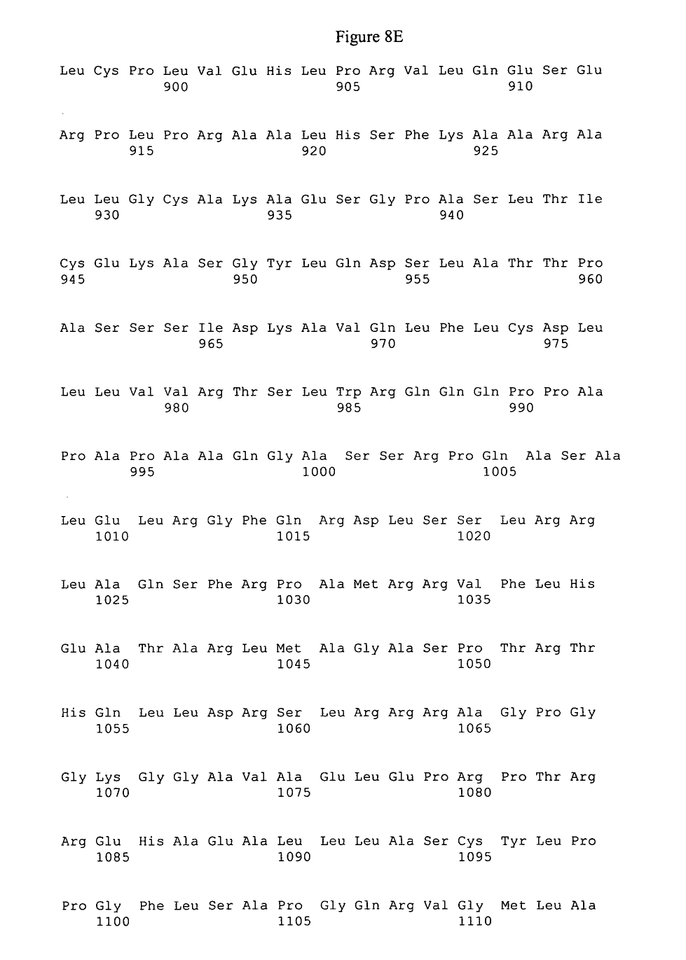

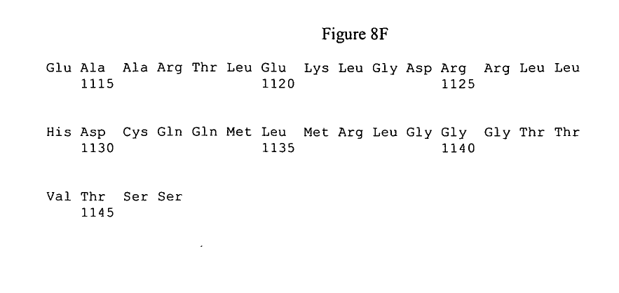

In yet another instance, the present invention relates to a portion of a human SREBP1 gene (FIGS. 1A-1B; SEQ ID NO:1) having a predicted nucleotide sequence according to FIGS. 7A-7B (SEQ ID NO:7) comprising single nucleotide polymorphisms at specific positions (e.g., nucleotide 1904 and nucleotide 1415). The reference nucleotide for this polymorphic form of SREBP1 is shown in FIGS. 1A-1B (SEQ ID NO:1) and the predicted variant nucleotide is shown in FIG. 7A-7B (SEQ ID NO:7). The variant nucleotide encodes the polypeptide shown in FIGS. 8A-8F (SEQ ID NO:8). In one embodiment, a nucleic acid molecule of the present invention comprises the variant (alternate) nucleotides at the polymorphic positions.

The single nucleotide polymorphisms described herein derive from genes that are associated with elevated plasma HDL levels and therefore the decreased incidence of cardiovascular disease. Specifically, the presence of a single nucleotide polymorphism of the gene described herein is associated with plasma HDL levels and may decrease a subject's susceptibility to acquiring cardiovascular disease.

The invention further provides allele-specific oligonucleotides that hybridize to a gene comprising a single nucleotide polymorphism or to the complement of the gene. Such oligonucleotides may hybridize to one polymorphic form of the nucleic acid molecules described herein but not to the other polymorphic form(s) of the sequence. Thus, such oligonucleotides can be used to determine the presence or absence of particular alleles of the polymorphic sequences described herein. These oligonucleotides can be probes or primers.

The invention further provides a method of analyzing a nucleic acid from an individual. The method determines which base is present at any one of the polymorphic sites disclosed herein. Optionally, a set of bases occupying a set of the polymorphic sites disclosed herein is determined. This type of analysis can be performed on a number of individuals, who are also tested (previously, concurrently or subsequently) for the presence of a given phenotype (e.g., elevated plasma HDL levels). The presence or absence of phenotype is then correlated with a base or set of bases present at the polymorphic site or sites in the subjects tested.

Thus, the invention further relates to a method of predicting the presence, absence, likelihood of the presence or absence, or severity of a particular phenotype or disorder (e.g., a cardiovascular disease, elevated plasma HDL levels) associated with a particular genotype (a SREBP1 SNP at position 1415, 1904 and combinations thereof, in a SREBP1 wildtype sequence (e.g., SEQ ID NO:1). The method comprises obtaining a nucleic acid sample from an individual and determining the identity of one or more bases (nucleotides) at polymorphic sites of nucleic acid molecules described herein, wherein the presence of a particular base is correlated with a specified phenotype or disorder, thereby predicting the presence, absence, likelihood of the presence or absence, or severity of the phenotype or disorder in the individual. The correlation between a particular polymorphic form of SREBP1 and a phenotype can thus be used in methods of diagnosis of that phenotype, as well as in the development of treatments for the phenotype.

### Definitions

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, more preferably ±5%, even more preferably ±1%, and still more preferably ±0.1% from the specified amount, as such variations are appropriate.

As used herein, the terms “agonist” and “activator” are synonymous and refer to an agent that supplements or potentiates the bioactivity of a functional SREBP1 gene or protein, or that supplements or potentiates the bioactivity of a naturally occurring or engineered non-functional SREBP1 gene or protein.

As used herein, the terms “amino acid” and “amino acid residue” are used interchangeably and mean any of the twenty naturally occurring amino acids. An amino acid is formed upon chemical digestion (hydrolysis) of a polypeptide at its 30 peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature abbreviations for amino acid residues are shown in tabular form presented herein above.

It is noted that all amino acid residue sequences represented herein by formulae have a left-to-right orientation in the conventional direction of amino terminus to carboxy terminus. In addition, the phrases “amino acid” and “amino acid residue” are broadly defined to include modified and unusual amino acids.

Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the term “antagonist” and “inhibitor” are synonymous and refer to an agent that decreases or inhibits the bioactivity of a functional SREBP1 gene or protein, or that decreases or inhibits the bioactivity of a naturally occurring or engineered non-functional SREBP1 gene or protein.

As used herein, the term “biological activity” means any observable effect flowing from a SREBP1 polypeptide. Representative, but non-limiting, examples of biological activity in the context of the present invention include modulation of plasma HDL levels in a subject and treatment and/or prevention of cardiovascular disease. The term “biological activity” also encompasses the ability to activate the expression of genes implicated in the synthesis and uptake of cholesterol, fatty acids, triglycerides, phospholipids and the NADPH co-factor required to synthesize these molecules.

As used herein, the terms “candidate substance” and “candidate compound” are used interchangeably and refer to a substance that is believed to interact with another moiety, for example a given ligand that is believed to interact with a complete, or a fragment of, a SREBP1 polypeptide, and which can be subsequently evaluated for such an interaction. Representative candidate substances or compounds include “xenobiotics”, such as drugs and other therapeutic agents, carcinogens and environmental pollutants, natural products and extracts, as well as “endobiotics”, such as steroids, fatty acids and prostaglandins. Other examples of candidate compounds that can be investigated using the methods of the present invention include, but are not restricted to, agonists and antagonists of a SREBP1 polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the terms “cardiovascular disease” and “cardiovascular condition” are used interchangeably and mean any known or unknown adverse or undesirable condition in a subject (e.g., a human) related to systemic or pulmonary circulation, or blood flow or composition that deviates from what is generally accepted as physiologically normal for a healthy subject (e.g., a healthy human). Examples of cardiovascular diseases include hyperlipidemia, hypercholesteremia, hypertension, arterial disease (intermittent claudication), venous disease (deep vein thrombosis), AMI, stroke, myocardio infarction, congestive heart failure, arrthymias, cardiomyopathy, atherosclerosis, arterialsclerosis, microvascular disease, embolism, thrombosis, pulmonary edema, palpitation, dyspnea, angina, hypotension, syncope, heart murmur, aberrant ECG, hypertrophic cardiomyopathy, the Marfan syndrome, sudden death, prolonged QT syndrome, congenital defects, cardiac viral infections, valvular heart disease, hypertension, abnormalities, such as arterio-arterial fistula, arteriovenous fistula, cerebral arteriovenous malformations, congenital heart defects, pulmonary atresia, and Scimitar Syndrome.

Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, cardiovascular syphilis, and cardiovascular tuberculosis.

Cardiovascular diseases further include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

As used herein, the terms “elevated HDL levels” and “elevated plasma HDL levels” are used interchangeably and mean a plasma HDL level that is above the HDL level in a normal healthy subject. Normal plasma HDL levels can vary with species and subject. For a human normal healthy subject, plasma HDL levels in the range of about 35 to about 40 mg/dL is generally accepted as physiologically normal. The term also encompasses a condition in which greater than 25 percent of a human subject's cholesterol is in the form of HDL, since in a healthy human subject about 25 percent or more of the cholesterol of the subject is in the form of HDL. Conditions in which plasma HDL levels are below normal levels for a subject, are included in the term “cardiovascular disease.” Therefore a condition in which plasma HDL levels fall below about 35 to about 40 mg/dL (e.g., in conditions such as Tangier disease, as well as others members of the above list) is an example of a cardiovascular disease.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “chimeric protein” and “fusion protein’ are used interchangeably and mean a fusion of a first amino acid sequence encoding a SREBP1 polypeptide with a second amino acid sequence defining a polypeptide domain foreign to, and not homologous with, a SREBP1 polypeptide. A chimeric protein can present a foreign domain that is found in an organism that also expresses the first protein, or it can be an “interspecies” or “intergenic” fusion of protein structures expressed by different kinds of organisms. In general, a fusion protein can be represented by the general formula X-SREBP1-Y, wherein SREBP1 represents a portion of the protein which is derived from a SREBP1 polypeptide, and X and Y are independently absent or represent amino acid sequences which are not related to a SREBP1 sequence in an organism, which includes naturally occurring mutants. Analogously, the term “chimeric gene” refers to a nucleic acid construct that encodes a “chimeric protein” or “fusion protein” as defined herein.

As used herein the term “complementary” means a nucleic acid sequence that is capable of base-pairing according to the standard Watson-Crick complementarity rules. These rules generally hold that the larger purines will always base pair with the smaller pyrimidines to form only combinations of Guanine paired with Cytosine (G:C) and Adenine paired with either Thymine (A:T) in the case of DNA, or Adenine paired with Uracil (A:U) in the case of RNA.

As used herein, the term “DNA segment” means a DNA molecule that has been isolated free of total genomic DNA of a particular species. In one embodiment, a DNA segment encoding a SREBP1 polypeptide refers to a DNA segment that comprises a sequence selected from the group consisting of SEQ ID NOs:1, 3, 5, 7, but can optionally comprise fewer or additional nucleic acids, yet is isolated away from, or purified free from, total genomic DNA of a source species. Included within the scope of the term “DNA segment” are DNA segments and smaller fragments of such segments, as well as recombinant vectors, including, for example, plasmids, cosmids, phages, viruses, and the like, and primers and probes, such as those represented in SEQ ID NOs:9-19.

As used herein, the terms “DNA sequence encoding a SREBP1 polypeptide” and “nucleotide encoding a SREBP1 polypeptide” are used interchangeably and can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the phrase “enhancer-promoter” means a composite unit that contains both enhancer and promoter elements. An enhancer-promoter is operatively linked to a coding sequence that encodes at least one gene product.

As used herein, the terms “expression” and “gene expression” are used interchangeably and generally refer to the cellular processes by which a polypeptide is produced from RNA.

As used herein, the term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As used herein, the term “hybridization” means the binding of a molecule (e.g., a probe molecule, such as a molecule to which a detectable moiety has been bound), to a target sample (e.g., a target nucleic acid).

As used herein, the term “hybridization techniques” refers to molecular biological techniques that involve the binding or hybridization of a probe to complementary sequences in a polynucleotide. Included among these techniques are northern blot analysis, Southern blot analysis, nuclease protection assay, etc.

As used herein, the terms “hybridization” and “binding” are used interchangeably in the context of probes and denatured DNA. Probes that are hybridized or bound to denatured DNA are aggregated to complementary sequences in the polynucleotide. Whether or not a particular probe remains aggregated with the polynucleotide depends on the degree of complementarity, the length of the probe, and the stringency of the binding conditions. The higher the stringency, the higher the degree of complementarity should be and/or the longer the probe.

As used herein, the term “intron” means a DNA sequence present in a given gene that is not translated into protein.

As used herein, the term “isolated” means a oligonucleotide sequence of interest that is substantially free of unwanted nucleic acids, proteins, lipids, carbohydrates or other materials with which the sequence of interest can be associated in vivo or in vitro, such association being either in cellular material or in a synthesis medium. The term can also be applied to polypeptides, in which case the polypeptide will be substantially free of nucleic acids, carbohydrates, lipids and other undesired polypeptides.

Thus, “isolated” material means that the material in question exists in a physical milieu distinct from that in which it occurs in nature, and thus is altered “by the hand of man” from its natural state. For example, an isolated nucleic acid of the present invention can be substantially isolated with respect to the complex cellular milieu in which it naturally occurs. In some instances, the isolated material can form a part of a composition (for example, a crude extract containing other substances), buffer system or reagent mix. In other circumstances, the material can be purified to essential homogeneity, for example as determined by PAGE or column chromatography such as HPLC.

As used herein, the term “linkage” means the tendency of genes, alleles, loci or genetic markers to be inherited together as a result of their location on the same chromosome. A degree of linkage can be measured by percent recombination between the two genes, alleles, loci or genetic markers.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein the terms “modulate” or “modulates,” and grammatical derivations thereof, refer to an increase or decrease in the amount, quality or effect of a particular activity, DNA, RNA, or protein. The definition of “modulate” or “modulates” encompasses agonists and/or antagonists of a particular activity, DNA, RNA, or protein. Thus, as used herein, the term “modulate”, and grammatical derivations thereof, means an increase, decrease, or other alteration of any or all chemical and biological activities or properties mediated by a nucleic acid sequence, peptide or other molecule. The term “modulation” as used herein refers to both upregulation (i.e., activation or stimulation) and downregulation (i.e. inhibition or suppression) of a response by any mode of action.

As used herein, the terms “nucleic acid,” “nucleic acid molecule,” are used interchangeably and mean any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally-occurring nucleotides (e.g., deoxyribonucleotides and ribonucleotides, also referred to herein as “nucleotides” or “bases”), or analogs of naturally-occurring nucleotides (e.g., α-enantiomeric forms of naturally-occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocylcic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids,” which comprise naturally-occurring or modified nucleic acid bases attached to a polyamide backbone. Nucleic acids can be single stranded or double stranded. Additionally, the terms “nucleotide sequence”, “nucleic acid sequence”, “nucleic acid molecule” and “nucleic acid segment” are used interchangeably and are equivalent.

By employing the disclosure presented herein, a nucleic acid molecule of the present invention encoding a polypeptide of the present invention can be obtained using standard cloning and screening procedures, such as those for cloning cDNAs using mRNA as starting material.

As used herein, the terms “oligonucleotide” and “polynucleotide” are used interchangeably and mean a single- or double-stranded DNA or RNA sequence. An oligonucleotide or a polynucleotide can be naturally occurring or synthetic, but are typically prepared by synthetic means. In the context of the present invention, an “oligonucleotide” and a “polynucleotide” includes segments of DNA, and/or their complements, including any one of the polymorphic sites disclosed and described herein. The segments can be, for example, between and 250 bases, and, in some embodiments, between 5-10, 5-20, 10-20, 10-50, 20-50 or 10-100 bases in length. The polymorphic site can occur within any position of the segment. The segments can be derived from any of the allelic forms of DNA disclosed and described herein.

Thus, the terms “oligonucleotide” and “polynucleotide” refer to a molecule comprising two or more nucleotides. For example, an “oligonucleotide” or a “polynucleotide” can comprise a nucleotide sequence of a full length cDNA sequence, including the 5′ and 3′ untranslated sequences, the coding region, with or without a signal sequence, the secreted protein coding region, as well as fragments, epitopes, domains, and variants of the nucleic acid sequence. An “oligonucleotide” or a “polynucleotide” of the present invention also includes those polynucleotides capable of hybridizing, under stringent hybridization conditions (described herein), to sequences described herein, or the complement thereof.

Thus, an “oligonucleotide” or a “polynucleotide” of the present invention can comprise any polyribonucleotide or polydeoxribonucleotide, and can comprise unmodified RNA or DNA or modified RNA or DNA. Additionally, an “oligonucleotide” or a “polynucleotide” can comprise single- and double-stranded DNA, DNA that is a mixture of single- and double-stranded regions, single- and double-stranded RNA, RNA that is mixture of single- and double-stranded regions, hybrid molecules comprising DNA and RNA that can be single-stranded or, more typically, double-stranded or a mixture of single- and double-stranded regions. In addition, an “oligonucleotide” or a “polynucleotide” can comprise triple-stranded regions comprising RNA or DNA or both RNA and DNA. An “oligonucleotide” or a “polynucleotide” can also contain one or more modified bases or DNA or RNA backbones modified for stability or for other reasons. “Modified” bases include, for example, tritylated bases and unusual bases such as inosine. A variety of modifications can be made to DNA and RNA; thus, the terms “oligonucleotide” and “polynucleotide” embraces chemically, enzymatically, or metabolically modified forms.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest can be dependent, inter alia, upon the specific nature of the enhancer-promoter.

As used herein, the terms “organism”, “subject” and “patient” are used interchangeably and mean any organism referenced herein, including prokaryotes, though the terms preferably refer to eukaryotic organisms, more preferably to mammals, and most preferably to humans.

As used herein, a “polymorphic marker” or “polymorphic site” is a locus at which divergence occurs. In one embodiment of the present invention, the markers have at least two alleles, each occurring at frequency of greater than 1%, and more preferably greater than 10% or 20% of a selected population. A polymorphic locus can be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as “alternative” or “variant” alleles. The allelic form occurring most frequently in a selected population is sometimes referred to as the wild type form. Diploid organisms can be homozygous or heterozygous for allelic forms. As noted, a diallelic or biallelic polymorphism has two forms; a triallelic polymorphism has three forms.

As used herein, the terms “polymorphic position”, “polymorphic site”, “polymorphic locus”, and “polymorphic allele” are interchangeable and equivalent; these terms mean the location of a sequence identified as having more than one nucleotide represented at that location in a population comprising at least one or more individuals, and/or chromosomes.

As used herein, the term “polymorphism” means the occurrence of two or more genetically determined alternative sequences or alleles in a population. The variant sequence and the “original” sequence co-exist in the species' population. In some instances, such co-existence is in stable or quasi-stable equilibrium. A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence can exist and the polymorphism is thus said to be di-allelic. In other cases, the species' population can contain multiple alleles and the polymorphism is termed tri-allelic, etc. A single gene can have multiple different unrelated polymorphisms. For example, it may have a di-allelic polymorphism at one site and a multi-allelic polymorphism at another site.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product or an amino acid sequence.

Thus, a polypeptide of the present invention can comprise amino acids joined to each other by peptide bonds or modified peptide bonds, i.e., peptide isosteres, and can contain amino acids other than the 20 gene-encoded amino acids. A polypeptide can be modified by either natural processes, such as by posttranslational processing, or by chemical modification techniques which are well known in the art. Such modifications are described in basic texts and in more detailed monographs, as well as in research literature known to those of ordinary skill in the art. Modifications can occur anywhere in a polypeptide, including the peptide backbone, the amino acid side-chains and the amino or carboxyl termini. The same type of modification can be present in the same or varying degrees at several sites in a given polypeptide. Also, a given polypeptide can contain many types of modifications. A polypeptide can be branched, for example, as a result of ubiquitination, or a polypeptide can be cyclic, with or without branching. Cyclic, branched, and branched cyclic polypeptides can result from posttranslation natural processes or can be made by synthetic methods. Representative modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, PEGylation, proteolytic processing, phosphorylation, prenylation, racemization, selenoylation, sulfation, transfer-RNA mediated addition of amino acids to proteins such as arginylation, and ubiquitination (see, e.g., Creighton, Proteins: Structures and Molecular Principles, (2^{nd} ed.) W.H. Freeman & Co., New York, (1993); Posttranslational Covalent Modification Of Proteins, (Johnson, ed.), Academic Press, New York, pp. 1-12 (1983); Seifter et al., (1990) Method Enzymol. 182:626-646; Rattan et al., (1992) Ann. N.Y. Acad. Sci. 663:48-62, incorporated herein by reference).

As used herein, “a polypeptide having biological activity” refers to a polypeptide exhibiting activity similar, but not necessarily identical to, an activity of a SREBP1 polypeptide of the present invention, including mature forms, as measured in a particular biological assay, with or without dose dependency. In a case where dose dependency does exist, it need not be identical to that of the SREBP1 polypeptide, but rather substantially similar to the dose-dependence in a given activity as compared to a SREBP1 polypeptide of the present invention (i.e., a candidate polypeptide will exhibit greater activity or not more than about 25-fold less and, preferably, not more than about tenfold less activity, and most preferably, not more than about three-fold less activity relative to a SREBP1 polypeptide of the present invention).

As used herein, the term “primer” means a single-stranded oligonucleotide sequence that acts as a point of initiation for template-directed DNA synthesis under appropriate conditions (e.g., in the presence of four different nucleoside triphosphates and an agent for polymerization, such as DNA or RNA polymerase or reverse transcriptase) in a suitable buffer and at a suitable temperature. The appropriate length of a primer can depend on the intended use of the primer, but typically ranges from to nucleotides. Short primer molecules generally require cooler temperatures to form sufficiently stable hybrid complexes with the template. A primer need not reflect the exact sequence of the template, but is preferably sufficiently complementary to hybridize with a template. The term “primer site” refers to the area of the target DNA to which a primer hybridizes. The term “primer pair” refers to a set of primers comprising a 5′ (upstream) primer that hybridizes with the 5′ end of the DNA sequence to be amplified and a 3′ (downstream) primer that hybridizes with the complement of the 3′ end of the sequence to be amplified. A primer can comprise, for example, two or more deoxyribonucleotides or ribonucleotides, more than three deoxyribonucleotides or ribonucleotides, more than eight deoxyribonucleotides or ribonucleotides or at least about 20 deoxyribonucleotides or ribonucleotides of an exonic or intronic region. Such oligonucleotides can be, for example, between ten and thirty bases in length. In the context of the present invention, representative primers include, for example, SEQ ID NOs:11-12 and 15-19.

As used herein, the term “probe” refers to an oligonucleotide or short fragment of DNA designed, known or suspected to be sufficiently complementary to a sequence in a denatured nucleic acid to be probed and to be bound under selected stringency conditions.

Continuing, in one embodiment a probe is a hybridization probe; such a probe can be an oligonucleotide that binds, in a base-specific manner, to a complementary strand of nucleic acid. Such probes include peptide nucleic acids, such as described for example in Nielsen et al., (1991) Science 254:1497-1500. A probe can be of any length suitable for specific hybridization to the target nucleic acid sequence. The most appropriate length of the probe can vary, depending upon the hybridization method in which it is being used; for example, particular lengths might be more appropriate for use in microfabricated arrays, while other lengths might be more suitable for use in classical hybridization methods. Such optimizations will be known to the skilled artisan upon consideration of the present disclosure. Representative probes and primers can range from about 5 nucleotides to about 40 nucleotides in length. For example, probes and primers can be 5, 6, 8, 10, 12, 14, 16, 18, 20, 22, 24, 25, 26, or 40 nucleotides in length. In some embodiments, the probe or primer overlaps at least one polymorphic site occupied by any of the possible variant nucleotides. The nucleotide sequence can correspond to the coding sequence of the allele or to the complement of the coding sequence of the allele.

As used herein, the term “recombination” and grammatical derivations thereof, means a re-assortment of genes or characters in combinations different from what they were in the parents, in the case of linked genes by crossing over.

As used herein, the term “sequencing” means determining the ordered linear sequence of nucleic acids or amino acids of a DNA, RNA or protein target sample, using manual or automated laboratory techniques. Unless otherwise indicated, the nucleotide sequence of all DNA sequences disclosed herein can be determined by employing an automated DNA sequencer (such as the Model 373 available from Applied Biosystems, Inc., Foster City, Calif., USA); all amino acid sequences of polypeptides encoded by DNA molecules disclosed herein can be predicted by translating a DNA sequence or by performing a chemical operation on the amino acid sequence (e.g., Edman degradation), which can be performed on an automated system.

As used herein, the terms “SREBP1,” “SREBP1 gene” and “recombinant SREBP1 gene” mean a nucleic acid molecule comprising an open reading frame encoding a SREBP1 polypeptide of the present invention, including both exon and (optionally) intron sequences. The terms include homologues, including vertebrate homologues.

As used herein, the terms “SREBP1,” “SREBP1 gene product,” “SREBP1 protein,” “SREBP1 polypeptide,” and “SREBP1 peptide” are used interchangeably and mean peptides having amino acid sequences that are substantially identical to native amino acid sequences from an organism of interest and which are biologically active in that they comprise all or a part of the amino acid sequence of a SREBP1 polypeptide, or cross-react with antibodies raised against a SREBP1 polypeptide, or retain all or some of the biological activity (e.g., modulation of plasma HDL levels in a subject or treatment and/or prevention of cardiovascular disease) of the native amino acid sequence or protein. Such biological activity can include immunogenicity.

The terms “SREBP1 gene product”, “SREBP1 protein”, “SREBP1 polypeptide”, and “SREBP1 peptide” also include analogs of a SREBP1 polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those of ordinary skill in the art will appreciate that other analogs as yet undisclosed or undiscovered can be used to design and/or construct SREBP1 analogs. There is no need for a “SREBP1 gene product”, “SREBP1 protein”, “SREBP1 polypeptide”, or “SREBP1 peptide” to comprise all or substantially all of the amino acid sequence of a SREBP1 polypeptide gene product. Shorter or longer sequences are anticipated to be of use in the invention; shorter sequences are herein referred to as “segments”. Thus, the terms “SREBP1 gene product”, “SREBP1 protein”, “SREBP1 polypeptide”, and “SREBP1 peptide” also include fusion, chimeric or recombinant SREBP1 polypeptides and proteins comprising sequences of the present invention. Methods of preparing such proteins are disclosed herein and/or are known in the art.

As used herein, the term “stringent hybridization conditions”, in the context of nucleic acid hybridization experiments such as Southern and northern blot analysis, means a set of conditions under which single stranded nucleic acid sequences are unlikely to hybridize to one another unless there is substantial complementarity between the sequences. Stringent hybridization conditions can be both sequence- and environment-dependent. Longer sequences hybridize specifically at higher temperatures. An extensive guide to the hybridization of nucleic acids is found in Tijssen, Laboratory Techniques in Biochemistry and Molecular Biology-Hybridization with Nucleic Acid Probes, Elsevier, N.Y., N.Y., USA, (1993), part I, chapter 2. Generally, highly stringent hybridization and wash conditions are selected to be about 5° C. lower than the thermal melting point (Tₘ) for the specific sequence at a defined ionic strength and pH. Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe will hybridize specifically to its target subsequence, but to no other sequences.

Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | | Hyridization | Wash |
|---|---|---|---|---|
| Stringency | Polynucleotide | Hybrid Length | Temperature and | Temperature |
| Condition | Hybrid ± | (bp) ‡ | Buffer † | and Buffer † |
| A | DNA:DNA | > or equal to 50 | 65° C.; 1 × SSC -or- | 65° C.; |
| | | | 42° C.; 1 × SSC, 50% | 0.3 × SSC |
| | | | formamide | |
| B | DNA:DNA | <50 | Tb*; 1 × SSC | Tb*; 1 × SSC |
| C | DNA:RNA | > or equal to 50 | 67° C.; 1 × SSC -or- | 67° C.; |
| | | | 45° C.; 1 × SSC, 50% | 0.3 × SSC |
| | | | formamide | |
| D | DNA:RNA | <50 | Td*; 1 × SSC | Td*; 1 × SSC |
| E | RNA:RNA | > or equal to 50 | 70° C.; 1 × SSC -or- | 70° C.; |
| | | | 50° C.; 1 × SSC, 50% | 0.3 × SSC |
| | | | formamide | |
| F | RNA:RNA | <50 | Tf*; 1 × SSC | Tf*; 1 × SSC |
| G | DNA:DNA | > or equal to 50 | 65° C.; 4 × SSC -or- | 65° C.; 1 × SSC |
| | | | 45° C.; 4 × SSC, 50% | |
| | | | formamide | |
| H | DNA:DNA | <50 | Th*; 4 × SSC | Th*; 4 × SSC |
| I | DNA:RNA | > or equal to 50 | 67° C.; 4 × SSC -or- | 67° C.; 1 × SSC |
| | | | 45° C.; 4 × SSC, 50% | |
| | | | formamide | |
| J | DNA:RNA | <50 | Tj*; 4 × SSC | Tj*; 4 × SSC |
| K | RNA:RNA | > or equal to 50 | 70° C.; 4 × SSC -or- | 67° C.; 1 × SSC |
| | | | 40° C.; 6 × SSC, 50% | |
| | | | formamide | |
| L | RNA:RNA | <50 | Tl*; 2 × SSC | Tl*; 2 × SSC |
| M | DNA:DNA | > or equal to 50 | 50° C.; 4 × SSC -or- | 50° C.; 2 × SSC |
| | | | 40° C. 6 × SSC, 50% | |
| | | | formamide | |
| N | DNA:DNA | <50 | Tn*; 6 × SSC | Tn*; 6 × SSC |
| O | DNA:RNA | > or equal to 50 | 55° C.; 4 × SSC -or- | 55° C.; 2 × SSC |
| | | | 42° C.; 6 × SSC, 50% | |
| | | | formamide | |
| P | DNA:RNA | <50 | Tp*; 6 × SSC | Tp*; 6 × SSC |
| Q | RNA:RNA | > or equal to 50 | 60° C.; 4 × SSC -or- | 60° C.; 2 × SSC |
| | | | 45° C.; 6 × SSC, 50% | |
| | | | formamide | |
| R | RNA:RNA | <50 | Tr*; 4 × SSC | Tr*; 4 × SSC |
| ‡: The “hybrid length” is the anticipated length for the hybridized region(s) of the hybridizing polynucleotides. When hybridizing a polynucletotide of unknown sequence, the hybrid is assumed to be that of the hybridizing polynucleotide of the present invention. When polynucleotides of known sequence are hybridized, the | | | | |
| # hybrid length can be determined by aligning the sequences of the polynucleotides and identifying the region or regions of optimal sequence complementarity. Methods of aligning two or more polynucleotide sequences and/or determining the percent identity between two polynucleotide sequences are well known in the art (e.g., MEGALIGN program of the DNA*Star suite of programs (DNAStar Inc., Madison, Wisconsin), etc). | | | | |
| †: SSPE (1 × SSPE is 0.15M NaCl, 10 mM NaH2PO4, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. The hydridizations and washes may additionally include | | | | |
| # 5 × Denhardt's reagent, .5-1.0% SDS, 100 μg/ml denatured, fragmented salmon sperm DNA, 0.5% sodium pyrophosphate, and up to 50% formamide. | | | | |
| *Tb-Tr: The hybridization temperature for hybrids anticipated to be less than 50 base pairs in length should be 5-10° C. less than the melting temperature Tm of the hybrids there Tm is determined according to the following equations. For hybrids less than 18 base pairs in length, Tm(° C.) = 2(# of A + T bases) + 4(# of G + C bases). For | | | | |
| # hybrids between 18 and 49 base pairs in length, Tm(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = .165 M). | | | | |

±: The present invention encompasses the substitution of any one, or more DNA or RNA hybrid partners with either a PNA, or a modified polynucleotide. Such modified polynucleotides are known in the art and are more particularly described elsewhere herein.

Additional examples of stringency conditions for polynucleotide hybridization are provided, for example, in Sambrook et al., Molecular Cloning: A Laboratory Manual, (3^{rd} ed.) Cold Spring Harbor Laboratory Press, Cold Spring Harbor, N.Y., USA (2001), chapters 9 and 11, and Current Protocols in Molecular Biology, (Ausubel et al., eds.), Greene Publishing Associates and Wiley-Interscience, New York (2002) sections 2.10 and 6.3-6.4, which references are hereby incorporated by reference herein in their entireties.

Preferably, hybridizing polynucleotides have at least 70% sequence identity (more preferably, at least 80% identity; and most preferably at least 90% or 95% identity) with the polynucleotide of the present invention to which they hybridize, where sequence identity is determined by comparing the sequences of the hybridizing polynucleotides when aligned so as to maximize overlap and identity while minimizing sequence gaps. The determination of identity is well known in the art, and discussed more specifically elsewhere herein.

As used herein, the term “substantially pure” means that a polynucleotide or polypeptide of interest is substantially free of the sequences and molecules with which it is associated in its natural state, as well as those molecules used in a given isolation or synthesis procedure. The term “substantially free” means that the sample is at least 50%, preferably at least 70%, more preferably 80% and most preferably 90% free of the materials and compounds with which is it associated in nature or in a medium in which the polynucleotide or polypeptide of interest is synthesized.

As used herein, the term “transcription” means a cellular process involving the interaction of an RNA polymerase with a gene that directs the expression as RNA of the structural information present in the coding sequences of the gene. The process comprises, but is not limited to, the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “variant” means a polynucleotide or polypeptide differing from a SREBP1 polynucleotide or polypeptide of the present invention by one or more amino acids or nucleotides, but retaining essential properties thereof. Generally, variants are overall closely similar, and, in many regions, identical to a SREBP1 polynucleotide or polypeptide of the present invention. For example, a variant can comprise a “conservative” change, wherein a substituted amino acid has similar structural or chemical properties, e.g., replacement of leucine with isoleucine. More rarely, a variant can have a “nonconservative” change, e.g., replacement of a glycine with a tryptophan. Similar minor variations can also include amino acid deletions or insertions, or both. Guidance in determining which amino acid residues can be substituted, inserted, or deleted without abolishing biological or immunological activity may be found using computer programs well known in the art, for example, DNASTAR software (DNAStar Inc., Madison, Wis.).

Table 2 discloses some representative, but non-limiting properties that can be used as a guide when selecting a conservative mutation follows:

TABLE 2
Representative Conservative Amino Acid Substitutions

| Amino Acid Property | Amino Acid |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

As used herein, the term “vector” means a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of an SREBP1 polypeptide, as described further herein. Some representative vectors include, but are not limited to, pCMV (Invitrogen, Carlsbad, Calif., USA) pBluescript (Stratagene, La Jolla, Calif., USA), pUC18, pBLCAT3 (Luckow and Schutz, (1987) Nucleic Acids Res 15: 5490), pLNTK (Gorman et al., (1996) Immunity 5: 241-252), and pBAD/gIII (Stratagene, La Jolla, Calif.). A representative host cell is a human embryonic kidney cell, such as HEK293.

### General Considerations

In one aspect, the present invention pertains to the identification of polymorphisms that can predispose an individual to certain abnormal genetic and phenotypic conditions, although such conditions might not necessarily be undesirable. For example, a polymorphism in one or more genes that are expressed in liver can predispose an individual to a disorder of the liver or the condition might positively affect liver function. In another example, a polymorphism in one or more genes that are expressed in cardiovascular tissue might predispose an individual to a disorder of the heart and/or circulatory system or the polymorphism might impart a beneficial effect.

By altering an amino acid sequence, a SNP can alter the function of the encoded protein. Thus, the discovery of a SNP facilitates biochemical analysis of the variants and the development of assays to characterize the variants and the ability to screen for pharmaceutical that would interact directly with on or another form of the protein. A SNP (including a silent SNP) can also alter the regulation of the gene at the transcriptional or post-transcriptional level. Therefore, a SNP (including silent a SNP) can also enable the development of specific DNA, RNA, or protein-based diagnostics that detect the presence or absence of a polymorphism associated with a particular condition.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1 in 100 or 1 in 1000 members of a population).

A single nucleotide polymorphism sometimes arises due to substitution of one nucleotide for another at the polymorphic site. A substitution can be, for example, a transition, which is the replacement of one purine by another purine or one pyrimidine by another pyrimidine. A substitution can also be a transversion, which is the replacement of a purine by a pyrimidine or vice versa. Single nucleotide polymorphisms can also arise from a deletion of a nucleotide or an insertion of a nucleotide relative to a reference allele. Typically, a polymorphic site is occupied by a base other than the reference base. For example, where the reference allele contains the base “T” at the polymorphic site, the altered allele can contain a “C”, “G” or “A” at the polymorphic site.

Hybridizations are usually performed under stringent conditions, as described herein. Equivalent conditions can be determined by varying one or more of the parameters given as an example, as known in the art, while maintaining a similar degree of identity or similarity between the target nucleotide sequence and the primer or probe used.

### Features of the Polypeptides Encoded by Polynucleotides of the Present Invention

The polypeptides encoded by the polynucleotides of the present invention comprise several unique features. Some of these features are described in the following sections.

### Features of the Polypeptide Encoded by SEQ ID NO:1

The present invention relates to isolated nucleic acid molecules comprising, or alternatively, consisting of all or a portion of a wildtype human SREBP1 gene (e.g., wherein reference or wildtype allele is exemplified by a “G” at nucleotide 1415 and a “G” at position 1904 of SEQ ID NO:1). Preferred portions are at least 10, preferably at least 20, preferably at least 40, preferably at least 100, contiguous polynucleotides and comprise the reference allele at the nucleotide position(s) provided in SEQ ID NO:1.

The reference or wildtype human SREBP1 gene comprises the nucleotide sequence of SEQ ID NO:1, and encodes a SREBP 1 polypeptide comprising the amino acid sequence of SEQ ID NO:2. The reference or wildtype SREBP1 sequence encodes a SREBP1 polypeptide comprising valine residues at positions 417 and 580.

### Features of the Polypeptide Encoded by SEQ ID NO:3

The present invention also relates to isolated nucleic acid molecules comprising, or alternatively, consisting of, all or a portion of one or more variant alleles of a human SREBP1 gene (e.g., wherein the reference or wildtype allele is exemplified by a “G” at nucleotide 1415 of SEQ ID NO:1 and a variant allele is exemplified by an “A” at nucleotide 1415 (i.e., SEQ ID NO:3)). Preferred portions are at least 10, preferably at least 20, preferably at least 40, preferably at least 100, contiguous polynucleotides and comprise one or more alternate (or variant) allele(s) at nucleotide position(s) 1415 and/or 1904 of SEQ ID NO:1. The corresponding reference polypeptide comprises a valine residue at position 417 of the encoded polypeptide, while the variant polypeptide comprises a methionine residue at position 417 of the encoded polypeptide. Thus, in one embodiment, a variant SREBP1 polypeptide comprises a substitution of a methionine for a valine residue at position 417 in a SREBP1 polypeptide.

The present invention further relates to isolated gene products, e.g., polypeptides and/or proteins, which are encoded by a nucleic acid molecule comprising all or a portion of at least one or more variant allele(s) (e.g., SEQ ID NO:3) of the gene.

As described further herein and in the Examples, an association was found between a val/met polymorphism at codon 417 (i.e. nucleotide position no. 1415) and plasma HDL levels. Plasma HDL levels play a role in cardiovascular disease.

In one embodiment, the present invention relates to a method for predicting the likelihood that an individual will have a disorder, particularly a cardiovascular disorder, associated with one or more variant allele(s) at nucleotide position 1415 of a SREBP1 coding sequence (or diagnosing or aiding in the diagnosis of such a disorder) comprising the steps of obtaining a DNA sample from an individual to be assessed and determining the nucleotide present at said nucleotide position. The presence of the an “A” at said position (i.e., a variant allele) indicates that the individual has a greater likelihood of having a disorder associated therewith than an individual having a “G” at said position (i.e., a reference allele), or a greater likelihood of having more severe symptoms.

The present invention further relates to isolated proteins or polypeptides comprising, or alternatively, consisting of all or a portion of the encoded variant amino acid sequence of a human SREBP1 polypeptide having an “M” at position 580 (SEQ ID NO:3), wherein reference or wildtype human SREBP1 polypeptide is exemplified by a “V” at amino acid 417; SEQ ID NO:1. Preferred portions are at least 10, preferably at least 20, preferably at least 40, or preferably at least 100 contiguous polypeptides. The present invention further relates to isolated nucleic acid molecules encoding such polypeptides or proteins, as well as to antibodies that bind to such proteins or polypeptides.

Diseases, disorders and conditions that may be detected, diagnosed, identified, treated, prevented, and/or ameliorated by the SNPs and methods of the present invention (such as the nucleic acid sequence SEQ ID NO:3, which encodes a polypeptide having the sequence of SEQ ID NO:4) include, the following, non-limiting diseases and disorders: hyperlipidemia, hypercholesteremia, hypertension, arterial disease (intermittent claudication), venous disease (deep vein thrombosis), AMI, stroke, myocardio infarction, congestive heart failure, arrhthymias, cardiomyopathy, atherosclerosis, arterialsclerosis, microvascular disease, embolism, thrombosis, pulmonary edema, palpitation, dyspnea, angina, hypotension, syncope, heart murmur, aberrant ECG, hypertrophic cardiomyopathy, the Marfan syndrome, sudden death, prolonged QT syndrome, congenital defects, cardiac viral infections, valvular heart disease, hypertension, abnormalities, such as arterio-arterial fistula, arteriovenous fistula, cerebral arteriovenous malformations, congenital heart defects, pulmonary atresia, Scimitar Syndrome, heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, cardiovascular syphilis, cardiovascular tuberculosis, aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

### Features of the Polypeptide Encoded by SEQ ID NO:5

The present invention relates to isolated nucleic acid molecules comprising, or alternatively, consisting of, all or a portion of one or more variant alleles of a wildtype human SREBP1 gene (e.g., wherein the reference or wildtype allele is exemplified by a “G” at nucleotide 1904 of SEQ ID NO:1 and a variant allele is exemplified by an “A” at nucleotide 1904 of SEQ ID NO:1 (i.e., SEQ ID NO:5). The corresponding reference polypeptide comprises a valine residue at position 580 of the encoded polypeptide, while the variant polypeptide comprises a methionine residue at position 580 (represented in SEQ ID NO:6). Thus, in one embodiment, the variant SREBP1 polypeptide comprises a substitution of a methionine for a valine residue at position 580 in a SREBP1 polypeptide.

The present invention further relates to isolated gene products, e.g., polypeptides and/or proteins, which are encoded by a nucleic acid molecule comprising all or a portion of at least one or more variant allele(s) (e.g., SEQ ID NO:5) of the gene.

In one embodiment, the invention relates to a method for predicting the likelihood that an individual will have a disorder, particularly a cardiovascular disorder, associated with one or more variant allele(s) at nucleotide position 1904 of a SREBP1 coding sequence (or diagnosing or aiding in the diagnosis of such a disorder) comprising the steps of obtaining a DNA sample from an individual to be assessed and determining the nucleotide present at said nucleotide position. The presence of the an “A” at said position (i.e., a variant allele) indicates that the individual has lesser likelihood of having a cardiovascular disorder associated therewith than an individual having a “G” at said position (i.e., a reference allele), or a lesser likelihood of having more severe symptoms.

The present invention further relates to isolated proteins or polypeptides comprising, or alternatively, consisting of all or a portion of an encoded variant amino acid sequence of the human SREBP1 polypeptide having a “M” at amino acid 580 (SEQ ID NO:6), wherein reference or wildtype human SREBP1 polypeptide is exemplified by a “V” at amino acid 580 (SEQ ID NO:1). Preferred portions are at least 10, preferably at least 20, preferably at least 40, or preferably at least 100 contiguous polypeptides. The invention further relates to isolated nucleic acid molecules encoding such polypeptides or proteins, as well as to antibodies that bind to such proteins or polypeptides.

Diseases, disorders and conditions that may be detected, diagnosed, identified, treated, prevented, and/or ameliorated by the SNPs and methods of the present invention (such as the nucleic acid sequence SEQ ID NO:5, which encodes a olypeptide having the sequence of SEQ ID NO:6) include, the following, non-limiting diseases and disorders: hyperlipidemia, hypercholesteremia, hypertension, arterial disease (intermittent claudication), venous disease (deep vein thrombosis), AMI, stroke, myocardio infarction, congestive heart failure, arrhythmias, cardiomyopathy, atherosclerosis, arterialsclerosis, microvascular disease, embolism, thromobosis, pulmonary edema, palpitation, dyspnea, angina, hypotension, syncope, heart murmur, aberrant ECG, hypertrophic cardiomyopathy, the Marfan syndrome, sudden death, prolonged QT syndrome, congenital defects, cardiac viral infections, valvular heart disease, hypertension, abnormalities, such as arterio-arterial fistula, arteriovenous fistula, cerebral arteriovenous malformations, congenital heart defects, pulmonary atresia, Scimitar Syndrome, heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, cardiovascular syphilis, cardiovascular tuberculosis, aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

### Features of the Predicted Polypeptide Encoded by SEQ ID NO:7

The present invention relates to isolated nucleic acid molecules comprising, or alternatively, consisting of, all or a portion of one or more predicted variant alleles of a wildtype human SREBP1 gene, wherein the reference or wildtype allele is exemplified by a “G” at nucleotides 1415 and 1904 of SEQ ID NO:1 and a variant allele is exemplified by an “A” at nucleotides 1415 and 1904 of SEQ ID NO:1 (i.e., SEQ ID NO:7). The corresponding reference polypeptide comprises valine residues at positions 417 and 580 of the encoded polypeptide, while the predicted variant polypeptide comprises a methionine residue at positions 417 and 580 (represented in SEQ ID NO:8). Thus, in one embodiment, the predicted variant SREBP1 polypeptide comprises a substitution of a methionine for a valine residue at positions 417 and 580 in a SREBP1 polypeptide.

The present invention further relates to isolated gene products, e.g., polypeptides and/or proteins, which are encoded by a nucleic acid molecule comprising all or a portion of at least one or more predicted variant allele(s) (e.g., SEQ ID NO:7) of the gene.

In one embodiment, the present invention relates to a method for predicting the likelihood that an individual will have a disorder, particularly a cardiovascular disorder, associated with one or more predicted variant allele(s) at nucleotide positions 1415 and 1904 of a SREBP1 coding sequence (or diagnosing or aiding in the diagnosis of such a disorder) comprising the steps of obtaining a DNA sample from an individual to be assessed and determining the nucleotide present at said nucleotide position. The presence of the an “A” at said positions (i.e., a variant allele) indicates that the individual has a lesser likelihood of having a cardiovascular disorder associated therewith than an individual having a “G” at said positions (i.e., a reference allele), or a lesser likelihood of having more severe symptoms.

The present invention further relates to isolated proteins or polypeptides comprising, or alternatively, consisting of all or a portion of the encoded predicted variant amino acid sequence of the human SREBP1 polypeptide having an “M” at positions 417 and 580 (SEQ ID NO:8), wherein reference or wildtype human SREBP1 polypeptide is exemplified by a “V” at amino acids 417 and 580; SEQ ID NO:1. Preferred portions are at least 10, preferably at least 20, preferably at least 40, or preferably at least 100 contiguous polypeptides. The invention further relates to isolated nucleic acid molecules encoding such polypeptides or proteins, as well as to antibodies that bind to such proteins or polypeptides.

Diseases, disorders and conditions that might be detected, diagnosed, identified, treated, prevented, and/or ameliorated by the SNPs and methods of the present invention (such as the nucleic acid sequence SEQ ID NO:7, which encodes a polypeptide having the sequence of SEQ ID NO:8) include the following, non-limiting diseases and disorders: hyperlipidemia, hypercholesteremia, hypertension, arterial disease (intermittent claudication), venous disease (deep vein thrombosis), AMI, stroke, myocardio infarction, congestive heart failure, arrhythmias, cardiomyopathy, atherosclerosis, arterialsclerosis, microvascular disease, embolism, thromobosis, pulmonary edema, palpitation, dyspnea, angina, hypotension, syncope, heart murmur, aberrant ECG, hypertrophic cardiomyopathy, the Marfan syndrome, sudden death, prolonged QT syndrome, congenital defects, cardiac viral infections, valvular heart disease, hypertension, abnormalities, such as arterio-arterial fistula, arteriovenous fistula, cerebral arteriovenous malformations, congenital heart defects, pulmonary atresia, Scimitar Syndrome, heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, cardiovascular syphilis, cardiovascular tuberculosis, aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

### Production of the Polynucleotides and Polypeptides of the Present Invention

The following paragraphs describe some of methods and techniques that can be employed in the production of the various polynucleotides and polypeptides of the present invention.

### Production of a SREBP1 Polypeptide

The native and mutated SREBP1 polypeptides, and fragments thereof, of the present invention can be chemically synthesized in whole or part using techniques that are known in the art (see, e.g., Creighton, Proteins: Structures and Molecular Principles, (2^{nd} ed.) W.H. Freeman & Co., New York, (1993), incorporated herein by reference). Alternatively, methods that are known to those of ordinary skill in the art can be used to construct expression vectors comprising a partial or the entire native or mutated SREBP1 polypeptide coding sequence and appropriate transcriptional/translational control signals. These methods include in vitro recombinant DNA techniques, as described herein, synthetic techniques and in vivo recombination/genetic recombination (see, e.g., the techniques described in Sambrook et al., Molecular Cloning: A Laboratory Manual, (3^{rd} ed.) Cold Spring Harbor Laboratory Press, Cold Spring Harbor, N.Y., USA (2001) and Current Protocols in Molecular Biology, (Ausubel et al., eds.), Greene Publishing Associates and Wiley-Interscience, New York (2002), both of which are incorporated herein by reference.

A variety of host-expression vector systems can be utilized to express a SREBP1 coding sequence. These include, but are not limited to, microorganisms such as bacteria transformed with recombinant bacteriophage DNA, plasmid DNA or cosmid DNA expression vectors containing SREBP1 coding sequence; yeast transformed with recombinant yeast expression vectors containing a SREBP1 coding sequence; insect cell systems infected with recombinant virus expression vectors (e.g., baculovirus) containing a SREBP1 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a SREBP1 coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedrin promoter can be used. When cloning in mammalian cell systems, promoters derived from the genome of mammalian cells (e.g., metallothionein promoter) or from mammalian viruses (e.g., the adenovirus late promoter or the vaccinia virus 7.5K promoter) can be used. When generating cell lines that contain multiple copies of the tyrosine kinase domain DNA, SV40-, BPV- and EBV-based vectors can be used with an appropriate selectable marker. Representative methods of producing SREBP1 polypeptides are also disclosed herein.

In addition, polypeptides of the invention can be chemically synthesized using techniques known in the art (e.g., see Creighton, Proteins: Structures and Molecular Principles, (2^{nd} ed.) W.H. Freeman & Co., New York, (1993), and Hunkapiller et al., (1984) Nature 310:105-111, both of which are incorporated herein by reference). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

The present invention encompasses polypeptides that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications can be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides can also be modified with a detectable label, such as an enzymatic, fluorescent, isotopic or affinity label to allow for detection and isolation of the protein, the addition of epitope tagged peptide fragments (e.g., FLAG®, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Also provided by the present invention are chemically modified derivatives of the polypeptides of the present invention that can provide additional advantages such as increased solubility, stability and circulating time of the polypeptide, or decreased immunogenicity (see U.S. Pat. No. 4,179,337, incorporated herein by reference). The chemical moieties for derivitization can be selected from water soluble polymers such as polyethylene glycol, ethylene glycol/propylene glycol copolymers, carboxymethylcellulose, dextran, polyvinyl alcohol and the like. The polypeptides can be modified at random positions within the molecule, or at predetermined positions within the molecule and can include one, two, three or more attached chemical moieties.

The invention further encompasses chemical derivitization of the polypeptides of the present invention, for example where the chemical is a hydrophilic polymer residue. Exemplary hydrophilic polymers, including derivatives, can be those that include polymers in which the repeating units contain one or more hydroxy groups (polyhydroxy polymers), including, for example, poly (vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers can vary, and is generally about 50 to about 5,000,000, with polymers having a molecular weight of about 100 to about 50,000 being preferred in some embodiments. The polymers can be branched or unbranched. In other embodiments polymers have a molecular weight of about 150 to about 10,000, and in yet other embodiments, the polymers can have a molecular weights of about 200 to about 8,000.

For polyethylene glycol, a representative molecular weight is between about 1 kDa and about 100 kDa (the term “about,” as defined herein, indicating that in preparations of polyethylene glycol, some molecules will weigh more, some less, than the stated molecular weight) for ease in handling and manufacturing. Other sizes can be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

Additional representative polymers that can be used to derivitize polypeptides of the present invention, include, for example, poly (ethylene glycol) (PEG), poly (vinylpyrrolidine), polyoxomers, polysorbate and poly (vinyl alcohol). Among the various PEG polymers, those PEG polymers having a molecular weight of from about 100 to about 10,000 (e.g., from about 200 to about 8,000, with PEG 2,000, PEG 5,000 and PEG 8,000, which have molecular weights of 2,000, 5,000 and 8,000, respectively) can be particularly useful in some embodiments. Other suitable hydrophilic polymers, in addition to those exemplified above, will be readily apparent to one of ordinary skill in the art upon considering the present disclosure. Generally, the polymers used can include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art (see, e.g., EP 0 401 384 (coupling PEG to G-CSF) and Malik et al., (1992) Exp. Hematol. 20:1028-1035 (reporting PEGylation of GM-CSF using tresyl chloride), which are incorporated herein by reference). For example, polyethylene glycol can be covalently bound through amino acid residues via a reactive group, such as, a free amino or carboxyl group. Reactive groups are those to which an activated polyethylene glycol molecule can be bound. The amino acid residues having a free amino group can include lysine residues and the N-terminal amino acid residues; those having a free carboxyl group can include aspartic acid residues, glutamic acid residues and the C-terminal amino acid residue. Sulfhydryl groups can also be used as a reactive group for attaching the polyethylene glycol molecules. For therapeutic purposes attachment at an amino group, such as attachment at the N-terminus or lysine group, can be desirable.

One can specifically desire proteins chemically modified at the N-terminus. Using polyethylene glycol as an illustration of the present composition, one can select from a variety of polyethylene glycol molecules (by molecular weight, branching, etc.), the proportion of polyethylene glycol molecules to protein (polypeptide) molecules in the reaction mix, the type of PEGylation reaction to be performed, and the method of obtaining the selected N-terminally PEGylated protein. The method of obtaining the N-terminally PEGylated preparation (i.e., separating this moiety from other monoPEGylated moieties if necessary) can be by purification of the N-terminally PEGylated material from a population of PEGylated protein molecules. Selective proteins chemically modified at the N-terminus modification can be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved.

As with the various polymers exemplified above, it is contemplated that the polymeric residues can comprise functional groups in addition, for example, to those typically involved in linking the polymeric residues to the polypeptides of the present invention. Such functionalities include, for example, carboxyl, amine, hydroxy and thiol groups. These functional groups on the polymeric residues can be further reacted, if desired, with materials that are generally reactive with such functional groups and which can assist in targeting specific tissues in the body including, for example, diseased tissue. Exemplary materials which can be reacted with the additional functional groups include, for example, proteins, including antibodies, carbohydrates, peptides, glycopeptides, glycolipids, lectins, and nucleosides.

In addition to residues of hydrophilic polymers, a chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one of ordinary skill in the art, upon consideration of the present disclosure. Generally, saccharides that can be used for derivitization include saccharides that can be attached to the polypeptides of the invention via alkylation or acylation reactions.

Moreover, the invention also encompasses derivitization of the polypeptides of the present invention, for example, with a lipid (including cationic, anionic, polymerized, charged, synthetic, saturated, unsaturated, and any combination of the above, etc.) and/or a stabilizing agent.

The present invention encompasses derivitization of the polypeptides of the present invention, for example, with compounds that can serve a stabilizing function (e.g., to increase the polypeptides half-life in solution, to make the polypeptides more water soluble, to increase the polypeptides hydrophilic or hydrophobic character, etc.). Polymers useful as stabilizing materials can be of natural, semi-synthetic (modified natural) or synthetic origin.

Exemplary natural polymers include naturally occurring polysaccharides, such as, for exampje, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof Accordingly, suitable polymers include, for example, proteins, such as albumin, polyalginates, and polylactide-coglycolide polymers.

Exemplary semi-synthetic polymers include carboxymethylcellulose, hydroxymethylcellulose, hydroxypropylmethylcellulose, methylcellulose, and methoxycellulose.

Exemplary synthetic polymers include polyphosphazenes, hydroxyapatites, fluoroapatite polymers, polyethylenes (such as, for example, polyethylene glycol (including for example, the class of compounds referred to as Pluronics®, commercially available from BASF, Parsippany, N.J., USA), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof. Methods for the preparation of derivatized polypeptides of the invention which employ polymers as stabilizing compounds will be readily apparent to one of ordinary skill in the art, in view of the present disclosure, when coupled with information known in the art, such as that described and referred to in U.S. Pat. No. 5,205,290, incorporated herein by reference.

Moreover, the present invention encompasses additional modifications of the polypeptides of the present invention. Such additional modifications are known in the art, and are specifically provided, in addition to methods of derivitization, etc., in U.S. Pat. No. 6,028,066, incorporated herein by reference.

The polypeptides of the present invention can be in monomers or multimers (i.e., dimers, trimers, tetramers and higher multimers). Accordingly, the present invention relates to monomers and multimers of the polypeptides of the present invention, their preparation, and compositions (e.g., therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention can be homomers or heteromers. As used herein, the term “homomer” refers to a multimer containing only polypeptides corresponding to the amino acid sequence of SEQ ID NOs:2, 4, 6 and 8 (including fragments, variants, splice variants, and fusion proteins, corresponding to these polypeptides as described herein). These homomers can contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term “heteromer” refers to a multimer containing one or more heterologous polypeptides (i.e., polypeptides of different proteins) in addition to the polypeptides of the present invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of a polypeptide of the present invention can be the result of hydrophobic, hydrophilic, ionic and/or covalent associations and/or can be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations can involve one or more amino acid residues contained in the polypeptide sequence (e.g., a sequence recited in the Sequence Listing). In one instance, the covalent associations comprise cross-linking between cysteine residues located within the polypeptide sequences which interact in the native (i.e., naturally occurring) polypeptide. In another instance, the covalent associations are the consequence of chemical or recombinant manipulation. Alternatively, such covalent associations can involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, covalent associations of fusion proteins of the invention are between heterologous polypeptide sequence from another protein that is capable of forming covalently associated multimers, such as for example, osteoprotegerin (see, e.g., PCT Publication WO 98/49305). In another embodiment, two or more polypeptides of the invention are joined through peptide linkers. Examples include suitable peptide linkers described in U.S. Pat. No. 5,073,627, incorporated herein by reference. Proteins comprising multiple polypeptides of the invention separated by peptide linkers can be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., (1988) Science 240:1759), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT Publication WO 94/10308, incorporated herein by reference. In one example, a recombinant fusion protein comprising a polypeptide of the invention fused to a polypeptide sequence that dimerizes or trimerizes in solution is expressed in a suitable host cell, and the resulting soluble multimeric fusion protein is recovered from the culture supernatant using techniques known in the art.

In some cases, trimeric polypeptides of the invention might offer the advantage of enhanced biological activity. In that case, preferred leucine zipper moieties and isoleucine moieties are those that preferentially form trimers. One example is a leucine zipper derived from lung surfactant protein D (SPD), as described in Hoppe et al., (1994) FEBS Letters 344:191. Other peptides derived from naturally-occurring trimeric proteins can be employed in preparing trimeric polypeptides of the present invention.

In another example, proteins of the present invention can be associated by interactions involving a FLAG® (Sigma, St. Louis, Mo., USA) polypeptide sequence contained in a fusion protein of the present invention containing FLAG® polypeptide sequence. In a further embodiment, associations between proteins of the present invention are via interactions between heterologous polypeptide sequences contained in FLAG® fusion proteins of the present invention and anti-FLAG® antibody.

Multimers of the present invention can be generated using chemical techniques known in the art. For example, multimers of the present invention can be formed by chemically cross-linking polypeptides of the present invention using linker molecules and linker molecule length optimization techniques known in the art (see, e.g., U.S. Pat. No. 5,478,925, incorporated herein by reference). Additionally, multimers of the present invention can be generated using techniques known in the art to form one or more inter-molecule cross-links between the cysteine residues located within the sequence of the polypeptides desired to be contained in the multimer (see, e.g., U.S. Pat. No. 5,478,925). Further, polypeptides of the present invention can be modified by adding cysteine or biotin to the C terminus or N-terminus of the polypeptide, and techniques known in the art can be employed to generate multimers containing one or more of these modified polypeptides (see, e.g., U.S. Pat. No. 5,478,925). Additionally, techniques known in the art can be applied to generate liposomes containing the polypeptide components desired to be contained in the multimer of the invention (see, e.g., U.S. Pat. No. 5,478,925).

Alternatively, multimers of the present invention can be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925). In a specific embodiment, polynucleotides coding for a homodimer of the invention are generated by ligating a polynucleotide sequence encoding a polypeptide of the present invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No 5,478,925). In another embodiment, recombinant techniques described herein or otherwise known in the art are applied to generate recombinant polypeptides of the invention which contain a transmembrane domain (or hydrophobic or signal peptide) and can be incorporated by membrane reconstitution techniques into liposomes (see, e.g., U.S. Pat. No. 5,478,925).

A polypeptide of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Particularly, high performance liquid chromatography (“HPLC”) can be employed for purification.

Polypeptides of the present invention, including their secreted forms, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention can be glycosylated or can be non-glycosylated. In addition, polypeptides of the invention can also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

### Production of Nucleic Acids of the Present Invention

Nucleic acids of the present invention can be cloned, synthesized, recombinantly altered, mutagenized, or combinations thereof. Standard recombinant DNA and molecular cloning techniques used to isolate nucleic acids are well known in the art. Exemplary, non-limiting methods are described, for example, by Sambrook et al., Molecular Cloning: A Laboratory Manual, (3^{rd} ed.) Cold Spring Harbor Laboratory Press, Cold Spring Harbor, N.Y., (2001); by Silhavy et al., (1984) Experiments with Gene Fusions, Cold Spring Harbor Laboratory Press, Cold Spring Harbor, N.Y.,; by Current Protocols in Molecular Biology, (Ausubel et al., eds.), Greene Publishing Associates and Wiley-Interscience, New York (2002); and by Glover, (ed.) (1985) DNA Cloning: A Practical Approach, MRL Press, Ltd., Oxford, U.K, all of which are incorporated herein by reference. Site-specific mutagenesis to create base pair changes, deletions, or small insertions are also known in the art (see, e.g., Adelman et al., (1983) DNA 2:183; Sambrook et al., Molecular Cloning: A Laboratory Manual, (3^{rd} ed.) Cold Spring Harbor Laboratory Press, Cold Spring Harbor, N.Y., (2001)).

Sequences disclosed or detected by methods of the invention can be detected, subcloned, sequenced, and further evaluated by any measure well known in the art using any method usually applied to the detection of a specific DNA sequence including but not limited to dideoxy sequencing, PCR, oligomer restriction (Saiki et al., (1985) Bio/Technology 3:1008-1012, incorporated herein by reference), allele-specific oligonucleotide (ASO) probe analysis (Conner et al., (1983) Proc. Natl. Acad. Sci. U.S.A. 80:278, incorporated herein by reference), and oligonucleotide ligation assays (OLAs) (Landgren et. al., (1988) Science 241:1007, incorporated herein by reference). Molecular techniques for DNA analysis have been reviewed (Landgren et. al., (1988) Science 242:229-237, incorporated herein by reference) and can be employed in the present invention.

In other aspects, the present invention also relates to vectors comprising the polynucleotides of the present invention, host cells, and the production of polypeptides by recombinant techniques. The vector can be, for example, a phage, plasmid, viral, or retroviral vector. Retroviral vectors can be replication competent or replication defective. In the latter case, viral propagation generally will occur only in complementing host cells.

Polynucleotides can be joined with a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The polynucleotide insert should be operatively linked to an appropriate promoter, such as the phage λ PL promoter, the E. coli lac, trp, phoA and tac promoters, the SV40 early and late promoters and promoters of retroviral LTRs, to name a few. Other suitable promoters will be known to those of ordinary skill in the art. The expression constructs can further comprise sites for transcription initiation, termination, and, in the transcribed region, a ribosome binding site for translation. The coding portion of the transcripts expressed by the constructs can include a translation initiating codon at the beginning and a termination codon (UAA, UGA or UAG) appropriately positioned at the end of the polypeptide to be translated.

The expression vectors can include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Vectors preferred for use in bacteria include pQE70, pQE60 and pQE-9, (available from QIAGEN, Inc., Chatsworth, Calif., USA); pBluescript vectors, Phagescript vectors, pNH8A, pNH16a, pNH18A, pNH46A (available from Stratagene Cloning Systems, Inc., La Jolla, Calif., USA); and ptrc99a, pKK223-3, pKK233-3, pDR540, pRIT5 (available from Pharmacia, Piscataway, N.J., USA). Representative eukaryotic vectors include pWLNEO, pSV2CAT, pOG44, pXT1 and pSG available from Stratagene; and pSVK3, pBPV, pMSG and pSVL available from Pharmacia (Piscataway, N.J., USA). Representative expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlsbad, Calif., USA). Other suitable vectors will be readily apparent to one of ordinary skill in the art.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology, (2^{nd} ed.) Appleton & Lange, Norwalk, Conn. (1994). It is specifically contemplated that the polypeptides of the present invention can be expressed by a host cell lacking a recombinant vector.

In one embodiment, the yeast Pichia pastoris is used to express a polypeptide of the present invention in a eukaryotic system. Pichia pastoris is a methylotrophic yeast that can metabolize methanol as its sole carbon source. A main step in the methanol metabolization pathway is the oxidation of methanol to formaldehyde using 02. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris (see, Ellis et al., (1985) Mol. Cell. Biol. 5:1111-21; Koutz et al., (1989) Yeast 5:167-77; Tschopp et al., (1987) Nucl. Acids Res. 15:3859-76). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the present invention, as set forth herein, in a Pichia yeast system essentially as described in Pichia Protocols: Methods in Molecular Biology, (Higgins & Cregg, eds.), Humana Press, Totowa, N.J., USA (1998), incorporated herein by reference. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one of ordinary skill in the art will appreciate, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, can be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the present invention, and which activates, alters, and/or amplifies endogenous polynucleotides. For example, techniques known in the art can be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670; U.S. Pat. No. 5,733,761; PCT Publication No. WO 96/29411; PCT Publication No. WO 94/12650; Koller et al., (1989) Proc. Natl. Acad. Sci. USA 86:8932-8935; and Zijlstra et al., (1989) Nature 342:435-438, all of which are incorporated herein by reference).

### Preparation of SREBP1 Mutants

Throughout the present disclosure it is intended that the term “mutant” encompass not only mutants of a SREBP1 polypeptide but chimeric proteins generated using a SREBP1 as well. The generation of chimeric SREBP1 polypeptides is an aspect of the present invention. Such a chimeric polypeptide can comprise a SREBP1 polypeptide or a portion of a SREBP1, which is fused to a candidate polypeptide or a suitable region of the candidate polypeptide, for example a SREBP1 expressed in a species other than human. Thus, it is intended that the following discussion of mutant SREBP1 polypeptides apply mutatis mutandis to chimeric SREBP1 polypeptides and to structural equivalents thereof.

In accordance with the present invention, a mutation can be directed to a particular site or combination of sites of a wild-type SREBP1. For example, a residue having a location on, at or near the surface of the polypeptide can be replaced, resulting in an altered surface charge of one or more charge units, as compared to the wild-type SREBP1. Alternatively, an amino acid residue in a SREBP1 can be chosen for replacement based on its hydrophilic or hydrophobic characteristics.

Such mutants can be characterized by any one of several different properties as compared with the wild-type SREBP1. For example, such mutants can have an altered surface charge of one or more charge units, or can have an increase in overall stability. Other mutants can have altered substrate specificity in comparison with, or a higher specific activity than, a wild-type SREBP 1.

SREBP1 mutants of the present invention can be generated in a number of ways. For example, the wild-type sequence of a SREBP1 can be mutated at those sites identified using this invention as desirable for mutation (e.g., polymorphic sites), by means of oligonucleotide-directed mutagenesis or other conventional methods, such as deletion. Alternatively, mutants of a SREBP1 can be generated by the site-specific replacement of a particular amino acid with an unnaturally occurring amino acid. In addition, SREBP1 mutants can be generated through replacement of an amino acid residue, for example, a particular cysteine or methionine residue, with selenocysteine or selenomethionine. This can be achieved by growing a host organism capable of expressing either the wildtype or mutant polypeptide on a growth medium depleted of either natural cysteine or methionine (or both) but enriched in selenocysteine or selenomethionine (or both).

A mutation can be introduced into a DNA sequence coding for a SREBP1 using synthetic oligonucleotides. These oligonucleotides contain nucleotide sequences flanking the desired mutation sites. A mutation can be generated in the full-length DNA sequence of a SREBP1 or in any sequence coding for polypeptide fragments of a SREBP1.

According to the present invention, a mutated SREBP1 DNA sequence produced by the methods described herein, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired SREBP1 mutant coding sequence, an expression vector can also include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to a SREBP1 mutant coding sequence. For expression under the direction of the control sequences, a desired DNA sequence must be operatively linked to the control sequences; that is, the sequence must have an appropriate start signal in front of the DNA sequence encoding the SREBP1 mutant, and the correct reading frame to permit expression of that sequence under the control of the control sequences and production of the desired product encoded by that SREBP1 sequence must be maintained.

Any of a wide variety of well-known available expression vectors can be useful in the expression of a mutated SREBP1 coding sequence of the present invention. These expression vectors can be used in the techniques disclosed in the Examples and can include, for example, vectors comprising segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In one embodiment of this invention, the E. coli vector pRSETA, including a T7-based expression system, is employed.

In addition, any of a wide variety of expression control sequences—sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated SREBP1 polypeptides according to this invention. These hosts include, for example, bacteria, such as E. coli, Bacillus and Streptomyces, fungi, such as yeasts, plant cells, insect cells, such as Sf9 and Sf21cells, and transgenic host cells.

It is noted that not all expression vectors and expression systems function in the same way to express mutated DNA sequences of the present invention, and to produce modified SREBP1 polypeptides or SREBP1 mutants. Neither do all hosts function equally well with the same expression system. One of ordinary skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, can also be considered.

When selecting an expression control sequence, a variety of factors should also be considered. These include, for example, the relative strength of the system, its controllability and its compatibility with the DNA sequence encoding a modified SREBP1 polypeptide of this invention, with particular regard to the formation of potential secondary and tertiary structures.

Hosts should be selected by consideration of their compatibility with the chosen vector, the toxicity of a modified SREBP1 to them, their ability to express mature products, their ability to fold proteins correctly, their fermentation requirements, the ease of purification of a modified SREBP1 and safety. Within these parameters, one of skill in the art can select various vector/expression control system/host combinations that will produce useful amounts of a mutant SREBP1. A mutant SREBP1 produced in these systems can be purified by a variety of conventional steps and strategies, including those used to purify the wild-type SREBP1.

Once a SREBP1 mutation(s) has been generated in the desired location, such as a ligand binding site, the mutants can be tested for any one of several properties of interest. For example, mutants can be screened for an altered charge at physiological pH. This can be determined by measuring the mutant SREBP1 isoelectric point (pI) and comparing the observed value with that of the wild-type parent. Isoelectric point can be measured by gel-electrophoresis according to the method of Wellner (Wellner, (1971) Anal. Chem. 43: 597), for example. A mutant SREBP1 polypeptide containing a replacement amino acid located at the surface of the enzyme, as provided by the structural information of this invention, can lead to an altered surface charge and an altered pI.

In addition, a polynucleotide insert of the present invention can be operatively linked to “artificial” or chimeric promoters and transcription factors. Specifically, the artificial promoter can comprise, or alternatively consist of, any combination of cis-acting DNA sequence elements that are recognized by trans-acting transcription factors. In one embodiment, the cis acting DNA sequence elements and trans-acting transcription factors are operable in mammals. Further, the trans-acting transcription factors of such “artificial” promoters can also be “artificial” or chimeric in design themselves and can act as activators or repressors to the “artificial” promoter.

### Design and Preparation of SREBP1 Variants

The present invention encompasses variants (e.g., allelic variants, orthologs, etc.) of the polynucleotide sequences disclosed herein in SEQ ID NOs:1, 3, 5, and 7, and the complementary strand thereto. The present invention also encompasses variants of the polypeptide sequences, and/or fragments therein, disclosed in SEQ ID NOs:2, 4, 6 and 8, a polypeptide encoded by the polynucleotide sequences in SEQ ID NOs: 1, 3, 5 and 7.

Thus, one aspect of the invention provides an isolated nucleic acid molecule comprising, or alternatively consisting of, a polynucleotide having a nucleotide sequence selected from the group consisting of: (a) a nucleotide sequence encoding a related polypeptide of the present invention having an amino acid sequence as shown in the Figures and/or in the Sequence Listing (described in SEQ ID NOs:2, 4, 6 and 8); (b) a nucleotide sequence encoding a mature related polypeptide of the present invention having the amino acid sequence as shown in the Figures and/or in the Sequence Listing (described in SEQ ID NO:2, 4, 6 and 8); (c) a nucleotide sequence encoding a biologically active fragment of a related polypeptide of the present invention having an amino acid sequence shown in the Figures and/or in the Sequence Listing (described in SEQ ID NO:2, 4, 6 and 8); (d) a nucleotide sequence encoding an antigenic fragment of a related polypeptide of the present invention having an amino acid sequence shown in the Figures and in the Sequence Listing (described in SEQ ID NO: 2, 4, 6 and 8); (e) a nucleotide sequence complimentary to any of the nucleotide sequences in (a), (b), (c), (d) and (e), above.

The present invention is also directed to polynucleotide sequences which comprise, or alternatively consist of, a polynucleotide sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d) and (e), above. Polypeptides encoded by these nucleic acid molecules are also encompassed by the invention. In another embodiment, the present invention encompasses nucleic acid molecules which comprise, or alternatively, consist of a polynucleotide which hybridizes under stringent conditions, or alternatively, under lower stringency conditions, to a polynucleotide in (a), (b), (c), (d), and (e) above. Polynucleotides that hybridize to the complement of these nucleic acid molecules under stringent hybridization conditions or alternatively, under lower stringency conditions, are also encompassed by the present invention, as are polypeptides encoded by these polypeptides.

Another aspect of the present invention provides an isolated nucleic acid molecule comprising, or alternatively, consisting of, a polynucleotide having a nucleotide sequence selected from the group consisting of: (a) a nucleotide sequence encoding a related polypeptide of the present invention having an amino acid sequence as shown in the Figures and/or in the Sequence Listing and described herein; (b) a nucleotide sequence encoding a mature related polypeptide of the present invention having the amino acid sequence as shown in the Sequence Listing and described herein; (c) a nucleotide sequence encoding a biologically active fragment of a related polypeptide of the present invention having an amino acid sequence as shown in the Sequence Listing and described herein; (d) a nucleotide sequence encoding an antigenic fragment of a related polypeptide of the present invention having an amino acid sequence as shown in the Figures and/or in the Sequence Listing and descried herein; (e) a nucleotide sequence complimentary to any of the nucleotide sequences in (a), (b), (c), (d), or (e) above.

The present invention is also directed to nucleic acid molecules that comprise, or alternatively, consist of, a nucleotide sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), or (e) above.

The present invention is also directed to polypeptides that comprise, or alternatively consist of, an amino acid sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequences shown in SEQ ID NOs:2, 4, 6 and 8 of the Sequence Listing, a polypeptide sequence encoded by the nucleotide sequences shown in SEQ ID NOs:1, 3, 5 and 7, and/or polypeptide fragments of any of these polypeptides (e.g., those fragments described herein). Polynucleotides that hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence can include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. The query sequence can be an entire sequence referenced herein, the ORF (open reading frame), or any fragment specified as described herein.

As a practical matter, whether any particular nucleic acid molecule or polypeptide is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A representative method for determining the best overall match between a query sequence (a sequence of the present invention) and a subject sequence, also referred to as a global sequence alignment, can be determined using the CLUSTALW computer program (Thompson et al., (1994) Nucleic Acids Research 2 (22):4673-4680), which is based on the algorithm of Higgins et al., (1992) Computer Applications in the Biosciences (CABIOS) 8(2):189-191. In a sequence alignment the query and subject sequences are both DNA sequences. An RNA sequence can be compared by converting U's to T's. The result of said global sequence alignment is in percent identity. Representative parameters used in a CLUSTALW alignment of DNA sequences to calculate percent identify are: Matrix=BLOSUM, k-tuple=1, Number of Top Diagonals=5, Gap Penalty=3, Gap Open Penalty 10, Gap Extension Penalty=0, Scoring Method=Percent, Window Size=5 or the length of the subject nucleotide sequence, whichever is shorter.

If a subject sequence is shorter than a query sequence because of 5′ or 3′ deletions, not because of internal deletions, a manual correction can be made to the results. This is because the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score can be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence that are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence that is at least, for example, 95% “identical” to a query amino acid sequence of the present invention, it is intended that the amino acid sequence of the subject polypeptide is identical to the query sequence except that the subject polypeptide sequence can include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence can be inserted, deleted, or substituted with another amino acid. These alterations of the reference sequence can occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced herein (e.g., as shown in the Figures and/or SEQ ID NOs:2, 4, 6 and 8 of the Sequence Listing) can be determined conventionally using known computer programs. A preferred method for determining the best overall match between a query sequence (a sequence of the present invention) and a subject sequence, also referred to as a global sequence alignment, can be determined using the CLUSTALW computer program (Thompson et al., (1994) Nucleic Acids Research 2 (22):4673-4680), which is based on the algorithm of Higgins et al., (1992) Computer Applications in the Biosciences (CABIOS) 8(2):189-191. The result of said global sequence alignment is in percent identity.

If the subject sequence is shorter than the query sequence due to N- or C-terminal deletions, not because of internal deletions, a manual correction can be made to the results. This is because the CLUSTALW program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what can be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

The variants disclosed in, or identified or generated by the methods of, the present invention can contain alterations in the coding regions, non-coding regions, or both. In some embodiments of the present invention it can be desirable that polynucleotide variants containing alterations produce silent substitutions, additions, or deletions, but do not alter the properties or activities of the encoded polypeptide. Nucleotide variants produced by silent substitutions due to the degeneracy of the genetic code can also be desirable. Moreover, variants in which 5-10, 1-5, or 1-2 amino acids are substituted, deleted, or added in any combination are also desirable in some situations. Polynucleotide variants can be produced for a variety of reasons, e.g., to optimize codon expression for a particular host (change codons in the mRNA to those preferred by a bacterial host such as E. coli).

Naturally occurring variants are called “allelic variants”, and refer to one of several alternate forms of a gene occupying a given locus on a chromosome of an organism (Lewin, Genes VII, Oxford University Press, New York, USA (2000), incorporated herein by reference). These allelic variants can vary at either the polynucleotide and/or polypeptide level and are included in the present invention. Alternatively, non-naturally occurring variants can be produced by mutagenesis techniques or by direct synthesis.

Thus, the invention further includes polypeptide variants that show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., (1990) Science 247:1306-1310 (incorporated herein by reference), wherein it is indicated that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids can be important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used (Cunningham & Wells, (1989) Science 244:1081-1085). The resulting mutant molecules can then be tested for biological activity.

As stated by Cunningham & Wells, these two strategies have revealed that proteins are surprisingly tolerant of amino acid substitutions. It is further indicated which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly. Table 2 presented herein above discloses some representative, but non-limiting properties that can be used as a guide when selecting a conservative mutation.

Besides conservative amino acid substitution, variants of the present invention include, but are not limited to, the following: (i) substitutions with one or more of the non-conserved amino acid residues, where the substituted amino acid residues might or might not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fc fusion region peptide, or leader or secretory sequence, or a sequence facilitating purification. Such variant polypeptides will be within the scope of those of ordinary skill in the art, upon consideration of the present disclosure.

For example, polypeptide variants containing amino acid substitutions of charged amino acids with other charged or neutral amino acids can produce proteins with improved characteristics, such as less aggregation. Aggregation of pharmaceutical formulations both reduces activity and increases clearance due to the aggregate's immunogenic activity. (see, e.g., Pinckard et al., (1967) Clin. Exp. Immunol. 2:331-340; Robbins et al., (1987) Diabetes 36: 838-845; Cleland et al., (1993) Crit. Rev. Therapeutic Drug Carrier Systems 10:307-377.)

Moreover, the invention further comprises polypeptide variants created through the application of molecular evolution (“DNA shuffling”) methodology to the polynucleotides disclosed in SEQ ID NOs:1, 3, 5, and 7, and/or the cDNA encoding the polypeptides disclosed as SEQ ID NOs:2, 4, 6 and 8. Such DNA shuffling technology is known in the art (see, e.g., Stemmer, (1994) Proc. Natl. Acad. Sci. 91:10747, incorporated herein by reference).

A further embodiment of the present invention relates to a polypeptide which comprises the amino acid sequence of the present invention having an amino acid sequence which contains, for example, at least one amino acid substitution, but not more than 50 amino acid substitutions, for example, or, in another embodiment, not more than 40 amino acid substitutions, or, in a further embodiment, not more than 30 amino acid substitutions, or, in yet another embodiment, not more than 20 amino acid substitutions. In some situations, it can be desirable for a peptide or polypeptide to comprise an amino acid sequence of the present invention, which comprises at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), can be, for example, 1-5, 5-10, 5-25, 5-50, 10-50 or 50-150, conservative amino acid substitutions.

The present invention further provides modified forms of nucleic acid sequences and corresponding proteins. Starting nucleic acid sequences can comprise one of the sequences described in Figures and/or the Sequence Listing, and the polynucleotides encoding the polypeptides described in the Figures and/or Sequence Listing. Some nucleic acids encode full-length modified forms of proteins. Modified genes can be expressed in an expression vector in which a variant gene is operably linked to a native or other promoter. Commonly, the promoter is a eukaryotic promoter for expression in a mammalian cell. The transcription regulation sequences typically include a heterologous promoter and optionally an enhancer that is recognized by the host. The selection of an appropriate promoter, for example trp, lac, phage promoters, glycolytic enzyme promoters and tRNA promoters, depends in part on the host selected. Commercially available expression vectors can be used. Vectors can include host-recognized replication systems, amplifiable genes, selectable markers, host sequences useful for insertion into the host genome, and the like.

The technique employed in introducing the expression construct into a host cell can vary with the particular construction and the target host. Suitable techniques include fusion, conjugation, transfection, transduction, electroporation or injection; such techniques are known in the art. A wide variety of host cells can be employed for expression of a modified gene, both prokaryotic and eukaryotic. Representative host cells include bacteria such as E. coli, yeast, filamentous fungi, insect cells, mammalian cells, typically immortalized, e.g., mouse, CHO, human and monkey cell lines and derivatives thereof. Preferably, but not necessarily, host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

A protein so produced can be isolated by conventional means of protein biochemistry and purification to obtain a substantially pure product via a number of techniques and/or protocols for example those described in Jacoby, Method Enzymol. volume 104, Academic Press, New York, N.Y., USA (1984); Scopes, Protein Purification, Principles and Practice, (2^{nd} ed.), Springer-Verlag, New York, N.Y., USA (1987); and Guide to Protein Purification, (Deutscher, ed.), Method Enzymol. vol. 182 (1990), all of which are incorporated herein by reference. If the protein is secreted, it can be isolated from the supernatant in which the host cell is grown. If not secreted, the protein can be isolated from a lysate of the host cells.

The present invention further provides transgenic nonhuman animals capable of expressing an exogenous variant gene and/or having one or both alleles of an endogenous variant gene inactivated. Expression of an exogenous variant gene is often achieved by operably linking the gene to a promoter and optionally an enhancer, and microinjecting the construct into a zygote (see Hogan et al., Manipulating the Mouse Embryo, A Laboratory Manual, (2^{nd} ed.) Cold Spring Harbor Laboratory Press, Plainview, N.Y., USA (1994), incorporated herein by reference). Inactivation of endogenous variant genes can be achieved by forming a transgene in which a cloned variant gene is inactivated by insertion of a positive selection marker (see Capecchi, (1989) Science 244: 1288-1292). The transgene is then introduced into an embryonic stem cell, where it undergoes homologous recombination with an endogenous variant gene. Mice and other rodents are representative animals. Such animals provide useful drug screening systems.

In addition to substantially full-length polypeptides expressed by variant genes, the present invention includes biologically active fragments of the polypeptides, or analogs thereof, including organic molecules that simulate the interactions of the peptides. Biologically active fragments include any portion of the full-length polypeptide that confers a biological function on the variant gene product, including ligand binding, and antibody binding. Ligand binding includes binding by nucleic acids, proteins or polypeptides, small biologically active molecules, or large cellular structures.

Polyclonal and/or monoclonal antibodies that specifically bind to variant gene products but not to corresponding prototypical gene products are also provided. As described further herein, antibodies can be made by injecting mice or other animals with the variant gene product or synthetic peptide fragments thereof. Monoclonal antibodies are screened as are described, for example, in Harlow & Lane, Antibodies: A Laboratory Manual, Cold Spring Harbor Press, Cold Spring Harbor, N.Y., USA (1988); Goding, Monoclonal Antibodies: Principles and Practice: Production and Application of Monoclonal Antibodies in Cell Biology, Biochemistry and Immunology, (3^{rd} ed.) Academic Press, San Diego, Calif., USA (1996), all of which are incorporated herein. Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

### Polynucleotide and Polypeptide Fragments

The present invention is directed to polynucleotide fragments of the polynucleotides of the invention, in addition to polypeptides encoded therein by said polynucleotides and/or fragments.

In the present disclosure, a “polynucleotide fragment” means a short polynucleotide having a nucleic acid sequence which: is a portion of those sequences shown in SEQ ID NOs:1, 3, 5, 7 and 9-19 or the complementary strand thereto, or is a portion of a polynucleotide sequence encoding a polypeptide of SEQ ID NOs:2, 4, 6 and 8. The nucleotide fragments of the invention can comprise, for example, at least about 15 nt, at least about 20 nt, at least about 30 nt, at least about 40 nt, at least about 50 nt, at least about 75 nt, or at least about 150 nt in length. A fragment “at least 20 nt in length,” for example, comprises 20 or more contiguous bases from the nucleotide sequences shown in SEQ ID NOs:1, 3, 5, and 7. Consistent with the definition provided herein, in this context the term “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. In some cases, larger fragments (e.g., 50, 150, 500, 600, 2000 nucleotides) can be desirable.

Moreover, representative examples of polynucleotide fragments of the present invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of any of SEQ ID NOs:1, 3, 5 and 7, or the complementary strand thereto. Again, consistent with the definition provided herein, in this context the term “about” includes the particularly recited ranges, and ranges larger or smaller by several (5, 4, 3, 2, or 1) nucleotides, at either terminus or at both termini. In one embodiment, these fragments encode a polypeptide that has biological activity. In another embodiment, these polynucleotides can be used as probes or primers as disclosed herein. Also encompassed by the present invention are polynucleotides that hybridize to these nucleic acid molecules under stringent hybridization conditions or lower stringency conditions, as are the polypeptides encoded by these polynucleotides.

In the present invention, a “polypeptide fragment” refers to an amino acid sequence that is a portion of that described in SEQ ID NOs:2, 4, 6 and 8. Protein (polypeptide) fragments can be “free-standing,” or a component of a larger polypeptide of which the fragment forms a part or region, most preferably as a single continuous region. Representative examples of polypeptide fragments of the present invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. Polynucleotides encoding these polypeptides are also encompassed by the invention.

A polypeptide fragment can comprise the full-length protein. Other representative polypeptide fragments include the full-length protein having a continuous series of deleted residues from the amino or the carboxy terminus, or both. For example, any number of amino acids, for example ranging from 1 to about 60, can be deleted from the amino terminus of the full-length polypeptide. Similarly, any number of amino acids, for example ranging from 1 to about 30, can be deleted from the carboxy terminus of the full-length protein. Furthermore, any combination of the above amino and carboxy terminus deletions can be made. Similarly, polynucleotides encoding these polypeptide fragments are also within the scope of the present invention.

In other examples, polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions can be made and are within the scope of the present invention. Polypeptide fragments of SEQ ID NOs:2, 4, 6 and 8 falling within conserved domains are specifically contemplated by the present invention. Moreover, polynucleotides encoding these domains are also contemplated.

Other exemplary polypeptide fragments comprise biologically active fragments. Biologically active fragments are those exhibiting activity similar, but not necessarily identical, to an activity of the polypeptide of the present invention. The biological activity of the fragments can include an improved desired activity, or a decreased abnormal activity. Polynucleotides encoding these polypeptide fragments are also encompassed by the invention.

In one embodiment, the functional activity displayed by a polypeptide encoded by a polynucleotide fragment of the invention can be one or more biological activities typically associated with the full-length polypeptide of the invention. Some representative biological activities are: the fragment's ability to bind to at least one of the same antibodies which bind to the full-length protein, the fragment's ability to interact with at lease one of the same proteins which associate with the full-length protein, the fragment's ability to elicit at least one of the same immune responses as the full-length protein (i.e., to cause the immune system to create antibodies specific to the same epitope, etc.), the fragment's ability to associate with at least one of the same polynucleotides as the full-length protein, the fragment's ability to associate with a receptor of the full-length protein, the fragment's ability to associate with a ligand of the full-length protein, and the fragment's ability to multimerize with the full-length protein. One of ordinary skill in the art will appreciate, however, that some fragments can have biological activities that are desirable and inapposite to the biological activity of the full-length protein. The functional activity of polypeptides of the invention, including fragments, variants, derivatives, and analogs thereof can be determined by numerous methods available to those of ordinary skill in the art, some of which are described herein.

The present invention further encompasses polypeptides comprising, or alternatively consisting of, an epitope of the polypeptide having an amino acid sequence of SEQ ID NOs:2, 4, 6 and 8, or encoded by a polynucleotide that hybridizes to the complement of the sequence of SEQ ID NO:1, 3, 5, and 7 under stringent hybridization conditions or lower stringency hybridization conditions as defined herein. The present invention further encompasses polynucleotide sequences encoding an epitope of a polypeptide sequence of the present invention, polynucleotide sequences of the complementary strand of a polynucleotide sequence encoding an epitope of the invention, and polynucleotide sequences that hybridize to the complementary strand under stringent hybridization conditions or lower stringency hybridization conditions defined herein.

As used herein, the term “epitopes” refers to portions of a polypeptide having antigenic or immunogenic activity in an animal, for example a mammal, such as a human. In one embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described herein (see, e.g., Geysen et al., (1983) Proc. Natl. Acad. Sci. USA 81:3998-4002).

The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments that function as epitopes can be produced by any conventional means (see, e.g., Houghten, (1985) Proc. Natl. Acad. Sci. USA 82:5131-5135, further described in U.S. Pat. No. 4,631,211, incorporated herein by reference).

In some embodiments of the present invention, antigenic epitopes contain a sequence of at least 4, at least 5, at least 6, at least 7, at least 8, at least 9, at least 10, at least 11, at least 12, at least 13, at least 14, at least 15, at least 20, at least 25, at least 30, at least 40, at least 50, or between about 15 to about 30 amino acids. Representative polypeptides comprising immunogenic or antigenic epitopes can be at least 10, 15, 20, 25, 30, 35, 40, 45, 50, 55, 60, 65, 70, 75, 80, 85, 90, 95, or 100 amino acid residues in length or longer. Additional non-exclusive representative antigenic epitopes include the antigenic epitopes disclosed herein, as well as portions thereof. Antigenic epitopes are useful, for example, to raise antibodies, including monoclonal antibodies, which specifically bind the epitope. Exemplary antigenic epitopes include the antigenic epitopes disclosed herein, as well as any combination of two, three, four, five or more of these antigenic epitopes. Antigenic epitopes can be used as the target molecules in immunoassays (see, e.g., Wilson et al., (1984) Cell 37:767-778; Sutcliffe et al., (1983) Science 219:660-666).

Similarly, immunogenic epitopes can be used, for example, to induce antibodies according to methods well known in the art (see, e.g., Sutcliffe et al., (1983) Science 219:660-666; Wilson et al., (1984) Cell 37:767-778; and Bittle et al., (1985) J. Gen. Virol. 66:2347-2354). Representative immunogenic epitopes include the immunogenic epitopes disclosed herein, as well as any combination of multiple epitopes, such as two, three, four, five or more of these immunogenic epitopes. Polypeptides comprising one or more immunogenic epitopes can be presented for eliciting an antibody response together with a carrier protein, such as an albumin, to an animal system (such as rabbit or mouse), or, if the polypeptide is of sufficient length (e.g., at least about 25 amino acids), the polypeptide can be presented without a carrier. However, immunogenic epitopes comprising as few as 8 to 10 amino acids have been shown to be sufficient to raise antibodies capable of binding to, at the very least, linear epitopes in a denatured polypeptide (e.g., in western blotting).

Epitope-bearing polypeptides of the present invention can be employed to induce antibodies according to methods known in the art including, but not limited to, in vivo immunization, in vitro immunization, and phage display methods (see, e.g., Sutcliffe et al., (1983) Science 219:660-666; Wilson et al., (1984) Cell 37:767-778; and Bittle et al., (1985) J. Gen. Virol. 66:2347-2354). If in vivo immunization is used, animals can be immunized with free peptide; however, anti-peptide antibody titer can be boosted by coupling the peptide to a macromolecular carrier, such as keyhole limpet hemacyanin (KLH) or tetanus toxoid. For instance, peptides containing cysteine residues can be coupled to a carrier using a linker such as maleimidobenzoyl-N-hydroxysuccinimide ester (MBS), while other peptides can be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections might be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal can be increased by selection of anti-peptide antibodies, for instance, by adsorption to the peptide on a solid support and elution of the selected antibodies according to methods well known in the art.

As one of ordinary skill in the art will appreciate, and as discussed herein, the polypeptides of the present invention comprising an immunogenic or antigenic epitope can be fused to other polypeptide sequences. For example, the polypeptides of the present invention can be fused with the constant domain of immunoglobulins (IgA, IgE, IgG, IgM), or portions thereof (CH1, CH2, CH3, or any combination thereof and portions thereof) resulting in chimeric polypeptides. Such fusion proteins can facilitate purification and can increase half-life in vivo. This has been shown for chimeric proteins consisting of the first two domains of the human CD4-polypeptide and various domains of the constant regions of the heavy or light chains of mammalian immunoglobulins (see, e.g., EP 394,827; Traunecker et al., (1988) Nature 331:84-86). Enhanced delivery of an antigen across the epithelial barrier to the immune system has been demonstrated for antigens (e.g., insulin) conjugated to an FcRn binding partner such as IgG or Fc fragments (see, e.g., PCT Publication WO 96/22024 and PCT Publication WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone (see, e.g., Fountoulakis et al., (1995) J. Biochem. 270:3958-3964). Nucleic acids encoding the above epitopes can also be recombined with a gene of interest as an epitope tag (e.g., the hemagglutinin (HA) tag or FLAG® tag) to aid in detection and purification of the expressed polypeptide. For example, a system described by Janknecht et al. allows for the ready purification of non-denatured fusion proteins expressed in human cell lines (Janknecht et al., (1991) Proc. Natl. Acad. Sci. USA 88:8972-8976). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers.

### Antibodies

In one embodiment the present invention comprises an isolated antibody that binds specifically to a polypeptide comprising an amino acid sequence comprising one or more polymorphic positions and is derived from, or corresponds to, a sequence selected from the group consisting of: an amino acid sequence of SEQ ID NOs:2, 4, 6, and 8.

Another aspect of the present invention relates to antibodies and T-cell antigen receptors (TCR) which immunospecifically bind a polypeptide, polypeptide fragment, or variant of SEQ ID NOs:2, 4, 6 and 8, and/or an epitope, of the present invention (as determined by immunoassays known in the art for assaying specific antibody-antigen binding). Representative antibodies of the present invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody”, as used herein, refers to immunoglobulin molecules and immunologically active portions of immunoglobulin molecules, i.e., molecules that contain an antigen binding site that immunospecifically binds an a antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F (ab′)2 fragments) which are capable of specifically binding to protein. Fab and F (ab′)2 fragments lack the Fc fragment of intact antibody, clear more rapidly from the circulation of the animal or plant, and can have less non-specific tissue binding than an intact antibody (Wahl et al., (1983) J. Nucl. Med. 24:316-325). Thus, these fragments can be desirable in some circumstances, as well as the products of a Fab or other immunoglobulin expression library. Moreover, antibodies of the present invention include chimeric, single chain, and humanized antibodies.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected for its specificity for a particular protein. For example, antibodies raised to a protein with an amino acid sequence encoded by any of the nucleic acid sequences of the invention can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art (see, e.g., U.S. Pat. No. 5,260,203). For this approach, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially similar to the binding specificity and affinity of the light and heavy chain aggregate variable region of an antibody described herein.

In one example, the antibodies are human antigen-binding antibody fragments of the present invention include, but are not limited to, Fab, Fab′ and F (ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, can comprise the variable region(s) alone or in combination with the entirety or a portion of the following: hinge region, CH1, CH2, and CH3 domains. Also included in the invention are antigen-binding fragments further comprising any combination of variable region(s) with a hinge region, CH1, CH2, and CH3 domains. The antibodies of the present invention can be from any animal origin including birds and mammals. For example, antibodies can be derived from human, murine (e.g., mouse and rat), donkey, sheep rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described herein and, for example in, U.S. Pat. No. 5,939,598.

The antibodies of the present invention can be monospecific, bispecific, trispecific or of greater multispecificity. Multispecific antibodies can be specific for different epitopes of a polypeptide of the present invention or can be specific for both a polypeptide of the present invention as well as for a heterologous epitope, such as a heterologous polypeptide or solid support material (see, e.g., PCT Publications WO 93/17715; WO 92/08802; WO 91/00360; WO 92/05793; U.S. Pat. Nos. 4,474,893; 4,714,681; 4,925,648; 5,573,920; 5,601,819; Tutt et al., (1991) J. Immunol. 147:60-69; Kostelny et al., (1992) J. Immunol. 148:1547-1553).

Antibodies of the present invention can be described or specified in terms of the epitope(s) or portion(s) of a polypeptide of the present invention that they recognize or specifically bind. The epitope(s) or polypeptide portion(s) can be specified as described herein, e.g., by N-terminal and C-terminal positions, by size in contiguous amino acid residues, or listed in the Sequence Listing and/or Figures. Antibodies that specifically bind any epitope or polypeptide of the present invention can also be excluded. Therefore, the present invention comprises antibodies that specifically bind polypeptides of the present invention, and allows for the exclusion of the same.

Antibodies of the present invention can also be described or specified in terms of their cross-reactivity. Antibodies that do not bind any other analog, ortholog, or homologue of a polypeptide of the present invention form other aspects of the present invention. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention also form aspects of the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are aspects of the present invention. In one embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further aspects of the present invention comprise antibodies that bind polypeptides encoded by polynucleotides that hybridize to a polynucleotide of the present invention under stringent hybridization conditions (as described herein). Antibodies of the present invention can also be described or specified in terms of their binding affinity to a polypeptide of the invention. Representative binding affinities include those with a dissociation constant (or K_{d}) of less than about 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹1 M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The invention also provides antibodies that competitively inhibit binding of an antibody to an epitope of the invention as determined by any method known in the art for determining competitive binding, for example, the immunoassays described herein. In representative embodiments, the antibody competitively inhibits binding to the epitope by at least 95%, at least 90%, at least 85%, at least 80%, at least 75%, at least 70%, at least 60%, or at least 50%.

Antibodies of the present invention can act as agonists or antagonists of a polypeptide of the present invention. For example, the antibodies of the present invention can be specific for a single nucleotide polymorphism of any one of the polypeptides encoded by the polynucleotides of the present invention. In other examples, antibodies that are capable of specifically distinguishing between the variant and reference forms of a polypeptide of the present invention are disclosed. Such antibodies can form an aspect of a kit to identify variant or normal forms of a polypeptide, and hence determine whether a particular individual is at a higher or lower risk of being susceptible to cardiovascular disease.

Antibodies of the present invention can be used, for example, to purify, detect, and/or target a polypeptide of the present invention, in either or both in vitro and in vivo diagnostic and therapeutic methods. For example, the antibodies can be employed in immunoassays for qualitatively and quantitatively measuring levels of a polypeptide of the present invention in a biological sample (see, e.g., Harlow & Lane, Antibodies: A Laboratory Manual, Cold Spring Harbor Press, Cold Spring Harbor, N.Y., USA (1988)).

As described herein, the antibodies of the present invention can be used either alone or in combination with other compositions. The antibodies can further be recombinantly fused to a heterologous polypeptide at the N- or C-terminus or chemically conjugated (including covalently and non-covalently conjugations) to polypeptides or other compositions. For example, antibodies of the present invention can be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc. Additionally, the derivative can contain one or more non-classical amino acids.

The antibodies of the present invention can be generated by any suitable method known in the art.

The antibodies of the present invention can also comprise polyclonal antibodies. Methods of preparing polyclonal antibodies are (Harlow & Lane, Antibodies: A Laboratory Manual, Cold Spring Harbor Press, Cold Spring Harbor, N.Y., USA (1988)). For example, a polypeptide of the present invention can be administered to various host animals including, but not limited to, rabbits, mice, rats, etc. to induce the production of sera containing polyclonal antibodies specific for the antigen. The administration of the polypeptides of the present invention can entail one or more injections of an immunizing agent and, if desired, an adjuvant. Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also known in the art. For the purposes of the invention, “immunizing agent” is defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they can also be given intramuscularly, and/or intravenously). The immunizing agent can include polypeptides of the present invention or a fusion protein or variants thereof. Depending on the nature of the polypeptides (i.e., percent hydrophobicity, percent hydrophilicity, stability, net charge, isoelectric point etc.), it can be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to those of ordinary skill in the art. Examples of such immunogenic proteins include, but are not limited to keyhole limpet hemocyanin, serum albumin, bovine thyroglobulin, and soybean trypsin inhibitor. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which can be employed includes the MPL-TDM adjuvant (monophosphoryl lipid A, synthetic trehalose dicorynomycolate). The immunization protocol can be selected by one of ordinary skill in the art without undue experimentation.

The antibodies of the present invention can comprise monoclonal antibodies. Monoclonal antibodies can be prepared using hybridoma methods, such as those described by Kohler & Milstein (Kohler & Milstein, (1975) Nature, 256:495), in U.S. Pat. No. 4,376,110, in Harlow & Lane, Antibodies: A Laboratory Manual, Cold Spring Harbor Press, Cold Spring Harbor, N.Y., USA (1988), by Hammerling et al., Monoclonal Antibodies and T-Cell Hybridomas, Elsevier, New York, N.Y., USA (1981) (all of which are incorporated by reference), or other methods known in the art. Other examples of methods that can be employed for producing monoclonal antibodies include, but are not limited to, the human B-cell hybridoma technique (Kosbor et al., (1983) Immunology Today 4:72; Cole et al., (1983) Proc. Natl. Acad. Sci. USA 80:2026-2030), and the EBV-hybridoma technique (Cole et al., Monoclonal Antibodies And Cancer Therapy, Alan R. Liss, Inc., pp. 77-96, (1985); Nickoloff, Animal Cell Electroporation and Electrofusion Protocols, Humana Press, Totowa, N.J., (1995)). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include polypeptides of the present invention or a fusion protein thereof. Generally, peripheral blood lymphocytes (PBLs) can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (Godin, Monoclonal Antibodies: Principles and Practice, (3^{rd} ed.) Academic Press, San Diego, Calif., (1996), incorporated herein by reference). Immortalized cell lines are usually transformed mammalian cells, particularly myeloma cells of rodent, bovine and human origin. Rat or mouse myeloma cell lines can be employed. The hybridoma cells can be cultured in a suitable culture medium comprising one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some embodiments, immortalized cell lines are those that fuse efficiently, support stable high level expression of antibody by the selected antibody-producing cells, and are sensitive to a medium such as HAT medium. Immortalized cell lines can also be murine myeloma lines, which can be obtained, for instance, from the Salk Institute Cell Distribution Center, San Diego, Calif. and the American Type Culture Collection, Manassas, Va. As noted or implied throughout the specification, human myeloma and mouse-human heteromyeloma cell lines also have been described for the production of human monoclonal antibodies (Kozbor, (1984) J. Immunol. 133:3001; Brodeur et al., Monoclonal Antibody Production Techniques and Applications, Marcel Dekker, Inc., New York, N.Y., USA, pp. 51-63 (1987)).

The culture medium in which the hybridoma cells are cultured can then be assayed for the presence of monoclonal antibodies directed against the polypeptides of the present invention. The binding specificity of monoclonal antibodies produced by the hybridoma cells can then be determined by immunoprecipitation or by an in vitro binding assay, such as radioimmunoassay (RIA) or enzyme-linked immunoadsorbant assay (ELISA). Such techniques are known in the art. The binding affinity of the monoclonal antibody can, for example, be determined by the Scatchard analysis of Munson & Pollart (Munson & Pollart, (1980) Anal. Biochem. 107:220).

After the desired hybridoma cells are identified, the clones can be subcloned by limiting dilution procedures and grown by standard methods (Goding, Monoclonal Antibodies: Principles and Practice, (3^{rd} ed.) Academic Press, San Diego, Calif. (1996)). Suitable culture media for this purpose include, for example, Dulbecco's Modified Eagle's Medium and RPMI-1640. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

A variety of methods exist in the art for the production of monoclonal antibodies and thus, the present invention is not limited to their sole production in hydridomas. For example, the monoclonal antibodies can be made by recombinant DNA methods, such as those described in U.S. Pat. No. 4,816,567, incorporated herein by reference. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hybridoma cells of the invention can serve as one source of such DNA. Once isolated, the DNA can be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow & Lane, Antibodies: A Laboratory Manual, Cold Spring Harbor Press, Cold Spring Harbor, N.Y., USA (1988), and in Hammerling et al., Monoclonal Antibodies and T-Cell Hybridomas, Elsevier, New York, N.Y., USA (1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are known in the art and are discussed herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the American Type Culture Collection. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments that recognize specific epitopes can be generated by known techniques. For example, Fab and F(ab′)2 fragments of the present invention can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles that carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., (1995) J. Immunol. Methods 182:41-50; Ames et al., (1995) J. Immunol. Methods 184:177-186; Kettleborough et al., (1994) Eur. J. Immunol. 24:952-958; Persic et al., (1997) Gene 187 9-18; Burton et al., (1994) Advances in Immunology 57:191-280; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, incorporated herein by reference.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in: PCT Publication WO 92/22324; Mullinax et al., (1992) BioTechniques 12 (6):864-869; Sawai et al., (1995) AJRI 34:26-34; and Better et al., (1988) Science 240:1041-1043. Examples of techniques that can be used to produce single-chain Fvs and antibodies include those described in U.S. Pat. Nos. 4,946,778 and 5,258,498; Huston et al., (1991) Method Enzymol. 203:46-88; Shu et al., (1993) Proc. Natl. Acad. Sci. 90:7995-7999; and Skerra et al., (1988) Science 240:1038-1040.

For some uses, including in vivo use of antibodies in humans and in vitro detection assays, it might be desirable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, (1985) Science 229:1202; Oi et al., (1986) BioTechniques 4:214; Gillies et al., (1989) J. Immunol. Methods 125:191-202; U.S. Pat. Nos. 5,807,715; 4,816,567; and 4,816397.

Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions can be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., U.S. Pat. No. 5,585,089 and Riechmann et al., (1988) Nature 332:323). Antibodies can be humanized using a variety of techniques known in the art including, for example, CDR-grafting (EP 239,400; PCT Publication WO 91/09967; U.S. Pat. Nos. 5,225,539; 5,530,101; and 5,585,089), veneering or resurfacing (EP 592,106; EP 519,596; Padlan, (1991) Molecular Immunology 28 (4/5):489-498; Studnicka et al., (1994) Protein Engineering 7 (6):805-814; Roguska et al., (1994) Proc. Natl. Acad. Sci. USA 91:969-973), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., (1986) Nature 321:522-525; Reichmann et al., (1988) Nature 332:323-327; Verhoeyen et al., (1988) Science 239:1534-1536, incorporated herein by reference), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally can also comprise at least a portion of an immunoglobulin constant region (Fc), typically that of a human immunoglobulin (Jones et al., (1986) Nature 321:522-525; Riechmann et al., (1988) Nature 332:323-329 and Presta, (1992) Curr. Opin. Struct. Biol. 2:593-596).

Human antibodies can be made by a variety of methods known in the art including phage display methods described above using antibody libraries derived from human immunoglobulin sequences. See also, U.S. Pat. Nos. 4,444,887 and 4,716,111; and PCT Publications WO 98/46645, WO 98/50433, WO 98/24893, WO 98/16654, WO 96/34096, WO 96/33735, and WO 91/10741. The techniques of Cole et al. and Boerder et al. are also available for the preparation of human monoclonal antibodies (Cole et al., Monoclonal Antibodies and Cancer Therapy, Alan R. Liss, (1985); and Boerner et al., (1991) J. Immunol. 147 (1):86-95).

Human antibodies can also be produced using transgenic mice that are incapable of expressing functional endogenous immunoglobulins, but can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg & Huszar, (1995) Int. Rev. Immunol. 13:65-93. For a detailed discussion of this technology for producing human antibodies and human monoclonal antibodies and protocols for producing such antibodies, see, e.g., PCT Publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, incorporated herein by reference. In addition, companies such as Abgenix, Inc. (Fremont, Calif., USA), Genpharm (San Jose, Calif., USA), and Medarex, Inc. (Princeton, N.J., USA) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., (1992) Biotechnol. 10:779-783; Lonberg et al., (1994) Nature 368:856-859; Fishwild et al., (1996) Nature Biotechnol. 14:845-51; Neuberger, (1996) Nature Biotechnol. 14:826; Lonberg & Huszer, (1995) Int. Rev. Immunol. 13:65-93.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., (1988) Bio/technology 12:899-903).

Further, antibodies to the polypeptides of the invention can, in turn, be utilized to generate anti-idiotype antibodies that “mimic” polypeptides of the invention using techniques known in the art (see, e.g., Greenspan & Bona, (1989) FASEB J. 7 (5):437-444; and Nissinoff, (1991) J. Immunol. 147 (8):2429-2438). For example, antibodies that bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention can be bispecific antibodies. Bispecific antibodies are monoclonal, preferably human or humanized, antibodies that have binding specificities for at least two different antigens. In the present invention, one of the binding specificities can be directed towards a polypeptide of the present invention, the other can be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein & Cuello, (1983) Nature 305:537-539). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in PCT Publication WO 93/08829, and in Traunecker et al., (1991) EMBO J. 10:3655-3659.

Antibody variable domains with the desired binding specificities (antibody-antigen combining sites) can be fused to immunoglobulin constant domain sequences. The fusion can be with an immunoglobulin heavy-chain constant domain, comprising at least part of the hinge, CH2, and CH3 regions. It is can be desirable to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. Additional details of generating bispecific antibodies are disclosed in the art, for example, in Suresh et al., (1986) Method Enzymol. 121:210.

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (PCT Publications WO 91/00360; WO 92/20373; and in EP 03089). It is contemplated that the antibodies can be prepared in vitro using known methods in synthetic protein chemistry, including those involving crosslinking agents. For example, immunotoxins can be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Methods of Producing Antibodies

The antibodies of the present invention can be produced by any method known in the art for the synthesis of antibodies, in particular, by chemical synthesis or by recombinant expression techniques.

Recombinant expression of an antibody of the invention, or fragment, derivative or analog thereof, (e.g., a heavy or light chain of an antibody of the invention or a single chain antibody of the invention), can involve the construction of an expression vector containing a polynucleotide that encodes the antibody. Once a polynucleotide encoding an antibody molecule or a heavy or light chain of an antibody, or portion thereof (for example, containing the heavy or light chain variable domain), of the present invention has been obtained, a vector for the production of the antibody molecule can be produced by recombinant DNA technology using techniques known in the art. Thus, methods for preparing a protein by expressing a polynucleotide comprising an antibody encoding nucleotide sequence are described herein. Methods known to those of ordinary skill in the art can be used to construct expression vectors comprising antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors can include the nucleotide sequence encoding the constant region of the antibody molecule (see, e.g., PCT Publication WO 86/05807; PCT Publication WO 89/01036; and U.S. Pat. No. 5,122,464) and the variable domain of the antibody can be cloned into such a vector for expression of the entire heavy or light chain.

The expression vector can be transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In some embodiments for the expression of double-chained antibodies, vectors encoding both the heavy and light chains can be co-expressed in the host cell for expression of the entire immunoglobulin molecule, as detailed herein.

A variety of host-expression vector systems can be utilized to express the antibody molecules of the invention. Such host-expression systems represent vehicles by which coding sequences of interest can be produced and subsequently purified, and also represent cells which can, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or eukaryotic cells, such as human embryonic kidney cells can be employed in the expression of whole recombinant antibody molecule, are used for the expression of a recombinant antibody molecule. In one example, mammalian cells such as Chinese hamster ovary cells (CHO), in conjunction with a vector such as the major intermediate early gene promoter element from human cytomegalovirus is an effective expression system for antibodies (Foecking et al., (1986) Gene 45:101; Cockett et al., (1990) Bio/Technology 8:662).

In bacterial systems, a number of expression vectors can be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified can be desirable. Such vectors include, but are not limited, to the E. coli expression vector pUR278 (Ruther et al., (1983) EMBO J. 2:1791), in which the antibody coding sequence can be ligated individually into the vector in frame with the lac Z coding region so that a fusion protein is produced; pIN vectors (Inouye & Inouve, (1985) Nucleic Acids Res. 13:3101-3109; Van Heeke & Schuster, (1989) J. Biol. Chem. 24:5503-5509); and the like. pGEX vectors can also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (see, e.g., Logan & Shenk, (1984) Proc. Natl. Acad. Sci. USA 81:355-359). Specific initiation signals might also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., (1987) Method Enzymol. 153:51-544).

In addition, a host cell strain can be chosen that modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines that express the antibody molecule. Such engineered cell lines can be particularly useful in screening and evaluation of compounds that interact directly or indirectly with the antibody molecule.

A number of selection systems can be used, including but not limited to the herpes simplex virus thymidine kinase (Wigler et al., (1977) Cell 11:223), hypoxanthine-guanine phosphoribosyltransferase (Szybalska & Szybalski, (1962) Proc. Natl. Acad. Sci. USA 48:2026-2034), and adenine phosphoribosyltransferase (Lowy et al., (1980) Cell 22:817) genes can be employed in tk-, hgprt- or aprt- cells, respectively. Also, antimetabolite resistance can be used as the basis of selection for the following genes: dhfr, which confers resistance to methotrexate (Wigler et al., (1980) Proc. Natl. Acad. Sci. USA 77:357; O'Hare et al., (1981) Proc. Natl. Acad. Sci. USA 78:1527); gpt, which confers resistance to mycophenolic acid (Mulligan & Berg, (1981) Proc. Natl. Acad. Sci. USA 78:2072); neo, which confers resistance to the aminoglycoside G-418 (Clinical Pharmacy 12:488-505; Wu & Wu, (1991) Biotherapy 3:87-95; Tolstoshev, (1993) Ann. Rev. Pharmacol. Toxicol. 32:573-596; Mulligan, (1993) Science 260:926-932; and Morgan & Anderson, (1993) Ann. Rev. Biochem. 62:191-217; TIB TECH 11(5):155-215); and hygro, which confers resistance to hygromycin (Santerre et al., (1984) Gene 30:147). Methods commonly known in the art of recombinant DNA technology can be routinely applied to select the desired recombinant clone, and such methods are described, for example, in Current Protocols in Molecular Biology, (Ausubel et al., eds.), Greene Publishing Associates and Wiley-Interscience, New York (2002); Kriegler, Gene Transfer and Expression, A Laboratory Manual, W.H. Freeman, New York, N.Y., USA (1991); Current Protocols in Human Genetics, (Dracopoli et al., eds.) John Wiley & Sons, New York, N.Y., USA (2001); and Colberre-Garapin et al., (1981) J. Mol. Biol. 150:1.

The expression levels of an antibody molecule can be increased by vector amplification (see generally Bebbington & Hentschel, The Use of Vectors Based on Gene Amplification for the Expression of Cloned Genes in Mammalian Cells in DNA Cloning, vol. 3., Academic Press, New York, N.Y., USA (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., (1983) Mol. Cell. Biol. 3:257).

The host cell can be co-transfected with two expression vectors of the invention, the first vector encoding a heavy chain derived polypeptide and the second vector encoding a light chain derived polypeptide. The two vectors can contain identical selectable markers that enable equal expression of heavy and light chain polypeptides. Alternatively, a single vector can be used which encodes, and is capable of expressing, both heavy and light chain polypeptides. In such situations, the light chain should be placed before the heavy chain to avoid an excess of toxic free heavy chain (Proudfoot, (1986) Nature 322:52; Kohler, (1980) Proc. Natl. Acad. Sci. USA 77:2197). The coding sequences for the heavy and light chains can comprise cDNA or genomic DNA.

Once an antibody molecule of the invention has been produced by an animal, chemically synthesized, or recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, for example at least 10, 20, 30, 40, 50, 60, 70, 80, 90 or 100 amino acids of the polypeptide) of the present invention to generate fusion proteins. The fusion does not necessarily need to be direct, but can occur through linker sequences. The antibodies can be specific for antigens other than polypeptides (or portion thereof, preferably at least 10, 20, 30, 40, 50, 60, 70, 80, 90 or 100 amino acids of the polypeptide) of the present invention. For example, antibodies can be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention can also be used in vitro immunoassays and purification methods using methods known in the art. See e.g., PCT Publication WO 93/21232; EP 439,095; Naramura et al., (1994) Immunol. Lett. 39:91-99; U.S. Pat. No. 5,474,981; Gillies et al., (1992) Proc. Natl. Acad. Sci. USA 89:1428-1432; Fell et al., (1991) J. Immunol. 146:2446-2452.

The present invention further includes compositions comprising the polypeptides of the present invention fused or conjugated to antibody domains other than the variable regions. For example, the polypeptides of the present invention can be fused or conjugated to an antibody Fc region, or portion thereof. The antibody portion fused to a polypeptide of the present invention can comprise the constant region, hinge region, CH1 domain, CH2 domain, and CH3 domain or any combination of whole domains or portions thereof. The polypeptides can also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT Publications WO 96/04388 and WO 91/06570; Ashkenazi et al., (1991) Proc. Natl. Acad. Sci. USA 88:10535-10539; Zheng et al., (1995) J. Immunol. 154:5590-5600; and Vil et al., (1992) Proc. Natl. Acad. Sci. USA 89:11337-11341, incorporated herein by reference.

As discussed herein, the polypeptides corresponding to a polypeptide, polypeptide fragment, or a variant of SEQ ID NOs:2, 4, 6 and 8 can be fused or conjugated to the above antibody portions to increase the in vivo half life of the polypeptides or for use in immunoassays using methods known in the art. Further, polypeptides corresponding to SEQ ID NO: 2, 4, 6 and 8 can be fused, or conjugated, to the noted antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., (1988) Nature 331:84-86). The polypeptides of the present invention fused or conjugated to an antibody having disulfide-linked dimeric structures (due to the IgG) can also be more efficient in binding and neutralizing other molecules, than the monomeric secreted protein or protein fragment alone. (Fountoulakis et al., (1995) J. Biochem. 270:3958-3964). In many cases, the Fc part in a fusion protein is beneficial in therapy and diagnosis, and thus can result in, for example, improved pharmacokinetic properties. Alternatively, deleting the Fc part after the fusion protein has been expressed, detected, and purified, might be desired. For example, the Fc portion might hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (see, Bennett et al., (1995) J. Molecular Recognition 8:52-58; Johanson et al., (1995) J. Biol. Chem. 270:9459-94714.

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., Chatsworth, Calif., USA), among others, many of which are commercially available. As described in Gentz et al., (1989) Proc. Natl. Acad. Sci. USA 86:821-824, for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., (1984) Cell 37:767) and the FLAG® tag (Sigma, St. Louis, Mo., USA).

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or a fragment thereof) or indirectly, through an intermediate (such as, for example, a linker known in the art) using techniques known in the art (see, for example, U.S. Pat. No. 4,741,900 for metal ions that can be conjugated to antibodies for use as diagnostics according to the present invention). Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Further, an antibody or fragment thereof can be conjugated to a therapeutic moiety such as a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibodies can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are known (see, e.g., Arnon et al., in Monoclonal Antibodies And Cancer Therapy, (Reisfeld et al., eds.), Alan R. Liss, Inc., (1985) pp. 243-56; Hellstrom et al., in Controlled Drug Delivery, (2^{nd} ed.) (Robinson et al., eds.), Marcel Dekker, Inc. (1987) pp. 623-53; Thorpe, in Monoclonal Antibodies '84: Biological And Clinical Applications, (Pinchera et al., eds.), (1985) pp. 475-506; Monoclonal Antibodies For Cancer Detection And Therapy, (Baldwin et al., eds.), Academic Press, New York (1985) pp. 303-16; and Thorpe et al., (1982) Immunol. Rev. 62:119-58).

Alternatively, an antibody can be conjugated to a second antibody to form an antibody heteroconjugate as described in U.S. Pat. No. 4,676,980.

An antibody, with or without a therapeutic moiety conjugated to it, administered alone or in combination with cytotoxic factor(s) and/or cytokine(s) can be used as a therapeutic.

### Assays for Antibody Binding

The antibodies of the present invention can be assayed for immunospecific binding by any method known in the art. Some immunoassays that can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are known in the art (see, e.g., Current Protocols in Molecular Biology, (Ausubel et al., eds.), Greene Publishing Associates and Wiley-Interscience, New York (2002). Seveal exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of ordinary skill in the art will be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Current Protocols in Molecular Biology, (Ausubel et al., eds.), Greene Publishing Associates and Wiley-Interscience, New York (2002) at 10.16.1, incorporated herein by reference.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of ordinary skill in the art will be knowledgeable as to the parameters that can be modified to increase the signal detected and to reduce the background noise. For further discussion regarding western blot protocols see, e.g., Current Protocols in Molecular Biology, (Ausubel et al., eds.), Greene Publishing Associates and Wiley-Interscience, New York (2002) at 10.8.1, incorporated herein by reference.

ELISA procedures generally comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In an ELISA, the antibody of interest does not have to be conjugated to a detectable compound; instead, a second antibody (which recognizes the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well. One of ordinary skill in the art will be knowledgeable as to the parameters that can be modified to increase the signal detected as well as other variations of ELISAs known in the art. For further discussion regarding ELISAs see, e.g., Current Protocols in Molecular Biology, (Ausubel et al., eds.), Greene Publishing Associates and Wiley-Interscience, New York (2002) at 11.2.1, incorporated herein by reference.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

### Diagnosis and Imaging with Antibodies

Labeled antibodies, and derivatives and analogs thereof, that specifically bind to a variant or reference allele of a polypeptide of interest can be used for diagnostic purposes to detect, diagnose, or monitor diseases, disorders, and/or conditions associated with the aberrant (although not necessarily undesirable) expression and/or activity of a polypeptide of the present invention.

The present invention provides for the detection of aberrant expression of a polypeptide of interest, comprising (a) assaying the expression of a SREBP1 polypeptide comprising a nucleic acid sequence selected from the group consisting of SEQ ID NOs:3, 5 and 7 in cells or body fluid of an individual using one or more antibodies specific to the polypeptide interest; and (b) comparing the level of gene expression with a standard gene expression level, whereby an increase or decrease in the assayed polypeptide gene expression level compared to the standard expression level is indicative of aberrant expression.

The present invention therefore provides a diagnostic assay for diagnosing a condition, comprising (a) assaying the expression of a SREBP1 polypeptide comprising a nucleic acid sequence selected from the group consisting of SEQ ID NOs:3, 5 and 7 in cells or body fluid of an individual using one or more antibodies specific to the polypeptide interest; and (b) comparing the level of gene expression with a standard gene expression level, whereby an increase or decrease in the assayed polypeptide gene expression level compared to the standard expression level is indicative of a particular condition. With respect to cancer and/or cardiovascular disease, the presence of a relatively high amount of transcript in biopsied tissue from an individual can indicate a predisposition for the development of the disease, or can provide a means for detecting the disease prior to the appearance of actual clinical symptoms. A more definitive diagnosis of this type can allow health professionals to employ preventative measures or aggressive treatment earlier thereby preventing the development or further progression of the cancer and/or cardiovascular disease.

Antibodies of the invention can be used to assay protein levels in a biological sample using classical immunohistological methods known to those of skill in the art (see, e.g., Jalkanen et al., (1985) J. Cell. Biol. 101:976-985; Jalkanen et al., (1987) J. Cell. Biol. 105:3087-3096). Other antibody-based methods useful for detecting protein gene expression include immunoassays, such as the ELISA and the RIA. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin.

One aspect of the invention is the detection and diagnosis of a disease, disorder or condition associated with aberrant expression of a SREBP1 polypeptide comprising a nucleic acid sequence selected from the group consisting of SEQ ID NOs:3, 5 and 7 in an animal, for example a mammal (particularly a human). In one embodiment, a diagnosis comprises: (a) administering (for example, parenterally, subcutaneously, or intraperitoneally) to a subject an effective amount of a labeled molecule which specifically binds to of a SREBP1 polypeptide comprising a nucleic acid sequence selected from the group consisting of SEQ ID NOs:3, 5 and 7; (b) waiting for a time interval following the administering for permitting the labeled molecule to preferentially concentrate at sites in the subject where the polypeptide is expressed (and for unbound labeled molecule to be cleared to background level); (c) determining background level; and (d) detecting the labeled molecule in the subject, such that detection of labeled molecule above the background level indicates that the subject has a particular disease or disorder associated with aberrant expression of a SREBP1 polypeptide encoded by a nucleic acid sequence selected from the group consisting of SEQ ID NOs:3, 5 and 7. Background level can be determined by various methods including, comparing the amount of labeled molecule detected to a standard value previously determined for a particular system.

Those of ordinary skill in the art will recognize that the size of the subject and the imaging system used can affect the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody or antibody fragment will then preferentially accumulate at the location of cells which contain the specific protein. In vivo tumor imaging is described in Tumor Imaging: Radioimmunochemical Detection of Cancer, (Burchiel & Rhodes, eds.) Masson Publishing Inc., New York, N.Y., USA (1982), Chapter 13.

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In another embodiment, monitoring of a disease, disorder or condition is carried out by repeating the method for diagnosing the disease, disorder or condition; for example, one month after initial diagnosis, six months after initial diagnosis, one year after initial diagnosis, etc.

The presence of the labeled molecule can be detected in the patient using methods known in the art for in vivo scanning. These methods depend upon the type of label used. One of ordinary skill in the art will be able to determine the appropriate method for detecting a particular label. Methods and devices that can be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (U.S. Pat. No. 5,441,050). In another embodiment, the molecule is labeled with a fluorescent compound and is detected in the subject using a fluorescence responsive scanning instrument. In yet another embodiment, the molecule is labeled with a positron emitting metal and is detected in the patent using positron emission-tomography. In a further embodiment, the molecule is labeled with a paramagnetic label and is detected in a patient using magnetic resonance imaging (MRI).

### Kits Comprising Antibodies

The present invention provides kits comprising antibodies that can be used in the above methods. In one embodiment, a kit comprises an antibody of the invention, preferably a purified antibody, in one or more containers. In a specific embodiment, a kit of the present invention contains a substantially isolated polypeptide comprising an epitope that is specifically immunoreactive with an antibody included in the kit. A kit of the present invention can further comprise a control antibody that does not react with a SREBP1 polypeptide comprising a nucleic acid sequence selected from the group consisting of SEQ ID NOs:3, 5 and 7. In another specific embodiment, a kit of the present invention contains a means for detecting the binding of an antibody to a SREBP1 polypeptide comprising a nucleic acid sequence selected from the group consisting of SEQ ID NOs:3, 5 and 7 (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate).

In another specific embodiment of the present invention, a kit is a diagnostic kit for use in screening serum containing antibodies specific against proliferative and/or cancerous polynucleotides and polypeptides. Such a kit can include a control antibody that does not react with the polypeptide of interest. Such a kit can include a substantially isolated polypeptide antigen comprising an epitope that is specifically immunoreactive with at least one anti-polypeptide antigen antibody. Further, such a kit includes means for detecting the binding of the antibody to the antigen (e.g., the antibody can be conjugated to a fluorescent compound such as fluorescein or rhodamine, which can be detected by flow cytometry). In specific embodiments, a kit can include a recombinantly produced or chemically synthesized polypeptide antigen. The polypeptide antigen of the kit can also be attached to a solid support (e.g., a multiwelled plate or plastic pins).

In a more specific embodiment the detecting means of the above-described kit comprises a solid support to which the polypeptide antigen is attached. Such a kit can also include a non-attached reporter-labeled anti-human antibody. In this embodiment, binding of the antibody to the polypeptide antigen can be detected by binding of the reporter-labeled antibody.

In an additional embodiment, the present invention comprises a diagnostic kit for use in screening serum containing antigens of the polypeptide of the invention. The diagnostic kit comprises a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody can be a monoclonal antibody. The detecting means of the kit can include a second, labeled monoclonal antibody. Alternatively, or in addition, the detecting means can include a labeled, competing antigen.

In one diagnostic configuration, a test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme that is detected by incubating the solid phase in the presence of a suitable fluorometric, luminescent or colorimetric substrate (commercially available from, for example, Sigma, St. Louis, Mo., USA).

The solid surface reagent in the above assay can be prepared by employing known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with a biotinylated antigen(s).

Thus, the present invention provides an assay system or kit for carrying out this diagnostic method. The kit generally comprises a support with surface-bound recombinant antigens, and a reporter-labeled anti-human antibody for detecting surface-bound anti-antigen antibody.

### Representative Uses for Antibodies Directed Against a SREBP1 Polypeptide of the Present Invention

An antibody of the present invention has various utilities. For example, such antibodies can be used in a diagnostic assay to detect the presence of, or to quantify the amount of, a variant or reference form of a polypeptide of the present invention in a sample. A representative diagnostic assay can comprise at least two steps. In the first step, a sample is contacted with an antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (see, e.g., Arenkov et al., (2000) Anal. Biochem. 278(2):123-131), or a chromatography column, etc. In a second step, the amount of antibody bound to the substrate is quantitated. In another embodiment, the method can additionally comprise a first step of attaching an antibody, for example covalently, electrostatically, or reversibly, to a solid support, and a second step of subjecting the bound antibody to the sample, as described herein.

Various diagnostic assay techniques are known in the art, such as competitive binding assays, direct or indirect sandwich assays and immunoprecipitation assays conducted in either heterogeneous or homogenous phases (see, e.g., Zola, Monoclonal Antibodies: A Manual of Techniques, CRC Press, Inc., Boca Raton, Fla., USA (1987), pp. 147-158, incorporated herein by reference in its entirety). The antibodies used in the diagnostic assays can also be labeled with a detectable moiety. The detectable moiety is preferably adapted to produce, either directly or indirectly, a detectable signal. For example, a detectable moiety can be a radioisotope, such as ²H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety can be employed, including those methods described by Hunter et al., (1962) Nature 144:945; Dafvid et al., (1974) Biochem. 13:1014; Pain et al., (1981) J. Immunol. Method 40:219; and Nygren, (1982) J. Histochem. Cytochem. 30:407.

### Therapeutic/Prophylactic Administration and Compositions

The present invention provides methods of treatment, inhibition and prophylaxis by administration to a subject of an effective amount of a compound or pharmaceutical composition of the invention, for example an antibody of the present invention. In one embodiment, the compound is substantially purified (e.g., substantially free from substances that limit its effect or produce undesired side-effects). The subject is can be an animal, such as a mammal, including but not limited to animals such as cows, pigs, horses, chickens, cats, dogs, rabbits, mice, rats, etc. In one embodiment, a subject is a human.

Formulations and methods of administration that can be employed when the compound comprises a nucleic acid or an immunoglobulin are described above; additional appropriate formulations and routes of administration can be selected from among those described herein below. Further formulations and routes of administration will be known to those of ordinary skill in the art upon consideration of the present disclosure.

Various delivery systems are known and can be used to administer a compound of the present invention, regardless of whether the compound comprises an antibody or other moiety, e.g., encapsulation in liposomes, microparticles, microcapsules, recombinant cells capable of expressing the compound, receptor-mediated endocytosis (see, e.g., Wu & Wu, (1987) J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local. In addition, it can sometimes be desirable to introduce the pharmaceutical compounds or compositions of the invention into the central nervous system by any suitable route, including intraventricular and intrathecal injection; intraventricular injection can be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it might be desirable to administer the pharmaceutical compounds or compositions of the invention locally to the area in need of treatment; this can be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. When administering a protein, including an antibody, of the invention, care should be taken to use materials to which the protein does not absorb.

In another embodiment, a compound or composition can be delivered in a vesicle, in particular a liposome (see, e.g., Langer, (1990) Science 249:1527-1533; Treat et al., in Liposomes in the Therapy of Infectious Disease and Cancer, (Lopez-Berestein and Fidler, eds.), Alfred R. Liss, New York, N.Y., USA, (1989) pp. 353-365).

In yet another embodiment, the compound or composition can be delivered in a controlled release system. In one embodiment, a pump can be used (see Langer, (1990) Science 249:1527-1533; Sefton, (1987) CRC Crit. Ref Biomed. Eng. 14:201; Buchwald et al., (1980) Surgery 88:507; Saudek et al., (1989) N. Engl. J. Med. 321:574). In another embodiment, polymeric materials can be used (see Medical Applications of Controlled Release, (Langer & Wise, eds.), CRC Pres., Boca Raton, Fla., USA (1984); Controlled Drug Bioavailability, Drug Product Design and Performance, (Smolen and Ball, eds.), Wiley, New York, N.Y., USA (1984); Ranger & Peppas, (1983) J. Macromol. Sci. Rev. Macromol. Chem. 23:61; see also Levy et al., (1985) Science 228:190; During et al., (1989) Ann. Neurol. 25:351; Howard et al., (1989) J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, vol. 2, (Langer & Wise, eds.), CRC Pres., Boca Raton, Fla., USA (1984), pp. 115-138).

Other controlled release systems are discussed in the review by Langer (Langer, (1990) Science 249:1527-1533).

In a specific embodiment in which a compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; BIOLISTIC®, Dupont, Wilmington, Del., USA), or coating with lipids or cell-surface receptors or transfecting agents, or by administering it in linkage to a homeobox-like peptide which is known to enter the nucleus (see, e.g., Joliot et al., (1991) Proc. Natl. Acad. Sci. USA 88:1864-1868, etc.). Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be a desirable carrier or carrier component when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, (20^{th} ed.), Lippincott, Williams & Wilkins, Baltimore, Md., USA (2001), incorporated herein by reference.

Such compositions can contain a therapeutically effective amount of the compound, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration comprise solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a compound of the invention that will be effective in the treatment, inhibition and prevention of a disease or disorder associated with aberrant expression and/or activity of a polypeptide of the invention can be determined by standard clinical techniques. In addition, in vitro assays can optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses can be extrapolated from dose-response curves derived from in vitro or animal model test systems.

For antibodies, the dosage administered to a patient is typically 0.1 mg/kg to 100 mg/kg of the patient's body weight. In some cases, the dosage administered to a patient can be between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention can be reduced by enhancing uptake and tissue penetration (e.g., in the liver, kidney, etc.) of the antibodies by modifications such as, for example, lipidation.

The present invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Sequence Similarity and Identity and Functional Equivalents

As used herein, the term “substantially similar” means that a particular sequence varies from nucleic acid sequences of SEQ ID NO:1, 3, 5, 7 and 9-19 or the amino acid sequence of SEQ ID NOs:2, 4, 6, and 8 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

### Sequences that are Substantially Identical to a SREBP1 Mutant Sequence of the Present Invention

Nucleic acids that are substantially identical to a nucleic acid sequence of a SREBP1 mutant of the present invention, e.g. allelic variants, genetically altered versions of the gene, etc., bind to a SREBP1 mutant sequence under stringent hybridization conditions. By using probes, particularly labeled probes of DNA sequences, one of ordinary skill in the art can isolate homologous or related genes. The source of homologous genes can be any species.

Between various species, homologues can have substantial sequence similarity, i.e. at least 85% sequence identity between nucleotide sequences. Sequence similarity is calculated based on a reference sequence, which can be a subset of a larger sequence, such as a conserved motif, coding region, flanking region, etc. A reference sequence can be, for example, at least about 18 nucleotides (nt) long, or in another example, at least about 30 nucleotides long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) that are similar, divided by the total number of symbols in the shorter of the two sequences. Parameters for the GAP program can be, for example, the default parameters, which do not impose a penalty for end gaps. See, e.g., Schwartz & Dayhoff, in Atlas of Protein Structure 5 (Suppl. 3), National Biomedical Research Foundation, Washington D.C., USA (1978), pp. 353-358, and Gribskov et al., (1986) Nucl. Acid Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs:1, 3, 5, and 7; or (b) the DNA analog sequence is capable of hybridization with DNA sequences of (a) under stringent conditions and which encode a biologically active SREBP1 gene product; or (c) the DNA sequences are degenerate as a result of alternative genetic code to the DNA analog sequences defined in (a) and/or (b). Substantially identical analog proteins and nucleic acids can have, for example, between about 70% and 80%, or about 81% to about 90% or about 91% and 99% sequence identity with the corresponding sequence of the native protein or nucleic acid. Sequences having lesser degrees of identity but comparable biological activity are considered to be equivalents.

As described herein above, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, conditions can comprise, for example, a salt concentration of about 200 mM and temperature of about 45° C. One example of such stringent conditions is hybridization at 4×SSC, at 65° C., followed by washing in 0.1×SSC at 65° C. for one hour. Another example of a stringent hybridization scheme uses 50% formamide, 4×SSC at 42° C.

In contrast, nucleic acids having sequence similarity can be detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

### Complementarity and Hybridization to a SREBP1 Mutant Sequence

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As described herein, nucleic acid hybridization can be affected by such conditions as salt concentration, temperature, or organic solvents, in addition to the base composition, length of the complementary strands, and the number of nucleotide base mismatches between the hybridizing nucleic acids, as will be appreciated by those of ordinary skill in the art. Stringent temperature conditions generally comprise temperatures in excess of about 30° C., typically in excess of about 37° C., and or temperatures in excess of about 45° C. Stringent salt conditions will ordinarily comprise less than about 1,000 mM, less than about 500 mM, or less than about 200 mM. However, the combination of parameters can be more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., Molecular Cloning: A Laboratory Manual, (3^{rd} ed.) Cold Spring Harbor Laboratory Press, Cold Spring Harbor, N.Y., USA (2001).

### Functional Equivalents of a SREBP1 Mutant Nucleic Acid Sequence of the Present Invention

As used herein, the term “functionally equivalent codon” is used to refer to codons that encode the same amino acid, such as the ACG and AGU codons for serine. Nucleic acid sequences comprising SEQ ID NOs:1, 3, 5, and 7, and fragments thereof, which have functionally equivalent codons, are covered by the present invention. Thus, when referring to the sequence examples presented in SEQ ID NOs:1, 3, 5, and 7, substitution of functionally equivalent codons into the sequence examples of SEQ ID NOs:1, 3, 5, 7 is contemplated.

It will also be understood by those of ordinary skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences that can comprise, for example, various non-coding sequences flanking either of the 5′ or 3′ portions of the coding region or can include various internal sequences, i.e., introns, which are known to occur within genes.

### Functional Equivalents

The present invention envisions and includes functional equivalents of a SREBP1 polypeptide of the present invention (e.g., a polypeptide comprising a sequence selected from the group consisting of SEQ ID NO:2, 4, 6 and 8). The term “functional equivalent” refers to proteins having amino acid sequences that are substantially identical to the amino acid sequence of a SREBP1 of the present invention and which are capable of exerting a biological effect in that they are capable of, for example, modulating plasma HDL levels or cross-reacting with anti-SREBP1 mutant antibodies raised against a SREBP1 polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of a SREBP1 polypeptide of the present invention or its underlying nucleic acid sequence without appreciable loss of biological utility or activity.

Functionally equivalent polypeptides, as used herein, are polypeptides in which certain, but not most or all, of the amino acids can be substituted. Thus, when referring to the sequence examples presented in SEQ ID NOs:1, 3, 5 and 7 the present invention contemplates substitution of codons that encode functionally equivalent amino acids, as described herein, into the sequence examples of SEQ ID NOs:2, 4, 6 and 8, respectively.

Alternatively, functionally equivalent proteins or peptides can be created via the application of recombinant DNA technology, in which changes in the protein structure can be engineered, based on considerations of the properties of the amino acids being exchanged, e.g. substitution of Ile for Leu. Changes designed by man can be introduced through the application of site-directed mutagenesis techniques, e.g., to introduce improvements to the antigenicity of the protein or to test a mutant SREBP1 polypeptide of the present invention for the ability to modulate plasma HDL levels, or other activity, at the molecular level.

Amino acid substitutions, such as those which that might be employed in modifying a SREBP1 polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of ordinary skill in the art upon consideration of the present disclosure. It is implicit in the above discussion, however, that one of ordinary skill in the art can appreciate that a radical, rather than a conservative substitution is warranted in a given situation. Non-conservative substitutions in SREBP1 polypeptides of the present invention are also an aspect of the present invention.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 1-19. Recombinant vectors and isolated DNA segments can therefore variously include a SREBP1 polypeptide-encoding region itself, include coding regions bearing selected alterations or modifications in the basic coding region, or include larger polypeptides which nevertheless comprise a SREBP1 polypeptide-encoding region or can encode biologically functional equivalent proteins or polypeptides which have variant amino acid sequences. Biological activity of a SREBP1 polypeptide can be determined, for example, by ligand-binding assays known to those of ordinary skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length being a reflection of, for example, the ease of preparation and use in the intended recombinant DNA protocol. For example, nucleic acid fragments can be prepared which include a short stretch complementary to a nucleic acid sequence set forth in SEQ ID NOs: 1, 3, 5 and 7, such as about 10 nucleotides, and which are up to 10,000 or 5,000 base pairs in length. DNA segments with total lengths of about 4,000, about 3,000, about 2,000, about 1,000, about 500, about 200, about 100, about 50, and about 25 base pairs in length can also be employed.

The DNA segments of the present invention encompass biologically functional equivalents of mutant SREBP1 polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced via the application of site-directed mutagenesis techniques, e.g., to introduce improvements to the antigenicity of the protein or to test variants of a mutant SREBP1 of the present invention in order to examine a degree of small molecule binding activity, or other activity at the molecular level. Various site-directed mutagenesis techniques are known to those of ordinary skill in the art and can be employed in the present invention.

The present invention further encompasses fusion proteins and peptides wherein a variant SREBP1 coding region of the present invention is aligned within the same expression unit with other proteins or peptides having desired functions, such as for purification or immunodetection purposes.

Recombinant vectors form further aspects of the present invention. Particularly useful vectors are those in which the coding portion of the DNA segment is positioned under the control of a promoter. The promoter can be a promoter naturally associated with a SREBP1 gene, as can be obtained by isolating the 5′ non-coding sequences located upstream of the coding segment or exon, for example, using recombinant cloning and/or PCR technology and/or other methods known to those of ordinary skill in the art, in conjunction with the compositions disclosed herein.

In other embodiments, certain advantages can be gained by positioning a coding DNA segment under the control of a recombinant, or heterologous, promoter. As used herein, a recombinant or heterologous promoter is a promoter that is not normally associated with a SREBP1 gene in its natural environment. Such promoters can include promoters isolated from bacterial, viral, eukaryotic, or mammalian cells. Naturally, it can be a consideration to employ a promoter that effectively directs the expression of the DNA segment in the cell type chosen for expression. The use of promoter and cell type combinations for protein expression is generally known to those of skill in the art of molecular biology (see, e.g., Sambrook et al., Molecular Cloning: A Laboratory Manual, (3^{rd} ed.) Cold Spring Harbor Laboratory Press, Cold Spring Harbor, N.Y., USA (2001)). The promoters employed can be constitutive or inducible and can be used under the appropriate conditions to direct high level expression of the introduced DNA segment, such as is advantageous in the large-scale production of recombinant proteins or peptides. One example promoter system contemplated for use in high-level expression is a T7 promoter-based system.

### Representative Applications of the Nucleic Acids of the Present Invention

Embodiments of the present invention include an isolated nucleic acid molecule comprising a nucleotide sequence containing one or more polymorphic positions and is at least about 20, 25, 30, 35, 40, 45, or 50 contiguous nucleotides and is derived from a nucleotide sequence selected from the group consisting of SEQ ID NOs:1, 3, 5, 7.

Another embodiment comprises an isolated nucleic acid molecule comprising a nucleotide sequence containing at least one or more polymorphic positions and is at least about 150 contiguous nucleotides and is derived from a nucleotide sequence selected from the group consisting of SEQ ID NOs:1, 3, 5, 7.

Another embodiment comprises an isolated nucleic acid molecule comprising a nucleotide sequence comprising at least one or more polymorphic positions and is at least about 500 contiguous nucleotides and is derived from a nucleotide sequence selected from the group consisting of SEQ ID NOs:1, 3, 5, 7.

Another embodiment comprises an isolated nucleic acid molecule comprising a nucleotide sequence containing one or more polymorphic positions and corresponds to, or is derived from, the complete nucleotide sequence selected from the group consisting of SEQ ID NOs:1, 3, 5, and 7.

Another embodiment comprises an isolated nucleic acid molecule that hybridizes under stringent hybridization conditions to a nucleic acid molecule.

Another embodiment comprises an isolated nucleic acid molecule, wherein the nucleic acid is included in the nucleotide sequence of the complete open reading frame sequence encoded by a cDNA clone of the present invention.

The present invention also encompasses an isolated nucleic acid molecule, wherein the nucleotide sequence encodes a polypeptide of the present invention and has been optimized for expression of said polypeptide in a prokaryotic host.

The present invention also encompasses the identification of proteins, nucleic acids, or other molecules, that bind to polypeptides and polynucleotides of the present invention (for example, in a receptor-ligand interaction). The polynucleotides of the present invention can also be used in interaction trap assays (such as, for example, that described by Ozenberger and Young (Ozenberger & Young, (1995) Mol Endocrinol. 9(10):1321-29; and Ozenberger & Young, (1995) Ann. N. Y. Acad. Sci. 766:279-81, incorporated herein by reference).

The polynucleotide and polypeptides of the present invention are also useful as probes for the identification and isolation of full-length cDNAs and/or genomic DNA that correspond to the polynucleotides of the present invention, as probes to hybridize and discover novel, related DNA sequences, as probes for positional cloning of this or a related sequence, as probe to “subtract-out” known sequences in the process of discovering other novel polynucleotides, as probes to quantify gene expression, and as probes for microarrays.

Also, in other embodiments the present invention provides methods for further refining the biological function of the polynucleotides and/or polypeptides of the present invention.

Specifically, the invention provides methods of using the polynucleotides and polypeptides of the invention to identify orthologs, homologs, paralogs, variants, and/or allelic variants of the invention. Also provided are methods of using the polynucleotides and polypeptides of the present invention to identify the entire coding region of the invention, non-coding regions of the invention, regulatory sequences of the invention, and secreted, mature, pro-, prepro-, forms of the present invention (as applicable).

The present invention also comprises a method of detecting, in a biological sample comprising a nucleic acid, a nucleic acid molecule comprising a nucleotide sequence comprising at least one or more polymorphic positions and corresponds to, or is derived from, a nucleotide sequence selected from the group consisting of SEQ ID NOs:1, 3, 5, 7, the method comprising comparing a nucleotide of the sample with a reference sequence selected from the group and determining whether the sequence of the nucleic acid molecule in the sample comprises one or more polymorphic positions relative to the reference sequence.

In another embodiment, the above method can optionally be modified, wherein the step of comparing sequences comprises determining the extent of nucleic acid hybridization between the nucleic acid molecule(s) in the sample and the reference nucleic acid molecule. The nucleic acid molecules can comprise DNA molecules or RNA molecules.

The polynucleotides of the present invention can comprise an element of a recombinant construct. For example, in one embodiment the present invention comprises a method of making a recombinant vector comprising inserting any of the isolated nucleic acid molecule(s) of the present invention into a vector. Another representative embodiment comprises a recombinant vector produced by this method. Another representative embodiment comprises a method of making a recombinant host cell comprising introducing a vector of the present invention into a host cell, as well as the recombinant host cell produced by this method.

### Allelic and Variant Polynucleotides and Polypeptides

The determination of the polymorphic form(s) present in an individual at one or more polymorphic sites defined herein can be used in a number of methods.

In some embodiments, the polynucleotides and polypeptides of the present invention, including allelic and variant forms thereof, have uses which include, but are not limited to diagnosing individuals to identify whether a given individual has decreased susceptibility or risk for cardiovascular disease and/or elevated plasma HDL levels using the genotype assays of the present invention.

In another embodiment, the polynucleotides and polypeptides of the present invention, including allelic and variant forms thereof, either alone, or in combination with other polymorphic polynucleotides (haplotypes) are useful as genetic markers.

The polynucleotides and polypeptides of the present invention, including allelic and/or variant forms thereof, are useful for creating recombinant vectors and hosts cells for the expression of variant and mutant forms of the polypeptides of the present invention.

The polynucleotides and polypeptides of the present invention, including allelic and/or variant forms thereof, are useful for creating antagonists directed against these polynucleotides and polypeptides, particularly antibody antagonists, for diagnostic, and/or therapeutic applications.

Additionally, the polynucleotides and polypeptides of the present invention, including allelic and/or variant forms thereof, are useful for creating additional antagonists directed against these polynucleotides and polypeptides, which include, but are not limited to the design of antisense RNA, ribozymes, PNAs, recombinant zinc finger proteins (Wolfe et al., (2000) Structure, Fold, Des. 8(7):739-50; Kang et al., (2000) J. Biol, Chem. 275 (12):8742-8; Wang & Pabo, (1999) Proc. Natl. Acad. Sci. USA 96(17):9568-73; McColl et al., (1999) Proc. Natl. Acad. Sci. USA 96(17):9521-6; Segal et al., (1999) Proc. Natl. Acad. Sci. USA 96(6):2758-63; Wolfe et al., (1999) J. Mol. Biol. 285(5): 1917-34; Pomerantz et al., (1998) Biochem. 37(4):965-70; Leon & Roth, (2000) Mol. Biol. Res. 33(1):21-30; Berg & Godwin, (1997) Ann. Rev. Biophys. Biomol. Struct. 26:357-71), in addition to other types of antagonists which are either described elsewhere herein, or known in the art.

The polynucleotides and polypeptides of the present invention, including allelic and/or variant forms thereof, are useful for creating small molecule antagonists directed against the variant forms of these polynucleotides and polypeptides, preferably wherein such small molecules are useful as therapeutic and/or pharmaceutical compounds for the treatment, detection, prognosis, and/or prevention of a variety of diseases and/or disorders, such as cardiovascular diseases, and conditions related to plasma HDL levels.

Another representative embodiment comprises a composition of matter comprising isolated an nucleic acid molecule, wherein the nucleotide sequence of the nucleic acid molecule comprises a panel of at least two nucleotide sequences, wherein at least one sequence in the panel comprises one or more polymorphic positions and is derived from, or corresponds to, a sequence selected from the group consisting of: a nucleotide sequence of SEQ ID NOs:1, 3, 5, 7 and fragments thereof.

The polynucleotides and polypeptides of the present invention, including allelic and/or variant forms thereof, are useful for the treatment of cardiovascular disease, in addition to other diseases and/or conditions referenced elsewhere herein, through the application of gene therapy based regimens.

Additional uses of the polynucleotides and polypeptides of the present invention are provided herein.

### Forensics

A determination of which polymorphic forms occupy a set of polymorphic sites in an individual identifies a set of polymorphic forms that distinguishes the individual from other individuals. See generally, National Research Council, The Evaluation of Forensic DNA Evidence (Pollard et al., eds.) National Academy Press, Washington D.C., USA (1996). The more sites that are analyzed, the lower the probability that the set of polymorphic forms in one individual is the same as that in an unrelated individual. If multiple sites are analyzed, the sites can be unlinked. Thus, polymorphisms of the invention are often used in conjunction with polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful in forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

The probability that two random individuals have the same polymorphic or allelic form at a given polymorphic site is denoted “p(ID)”. In biallelic loci, four genotypes are possible: AA, AB, BA, and BB. If alleles A and B occur in a haploid genome of the organism with frequencies x and y, the probability of each genotype in a diploid organism is (see PCT Publication WO 95/12607):
Homozygote: p(AA)=x²
Homozygote: p(BB)=y²=(1−x)²
Single Heterozygote: p(AB)=p(BA)=xy=x(1−x)
Both Heterozygotes: p(AB+BA)=2xy=2x(1−x)

The probability of identity at one locus (i.e., the probability that two individuals, picked at random from a population will have identical polymorphic forms at a given locus) is given by the equation:
p(ID)=(x²)²+(2xy)²+(y²)².

These calculations can be extended for any number of polymorphic forms at a given locus. For example, the probability of identity p (m) for a 3-allele system where the alleles have the frequencies in the population of x, y and z, respectively, is equal to the sum of the squares of the genotype frequencies:
p(ID)=x⁴+(2xy)²+(2yz)²+(2xz)²+z⁴+y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus.
cum p(ID)=p(ID1)p(ID2)p(ID3) . . . p(IDn)
The cumulative probability of non-identity for n loci (i.e. the probability that two random individuals will be different at 1 or more loci) is given by the equation:
cum p(nonlD)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

### Paternity Testing

An object of paternity testing is to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the set of polymorphisms of the putative father, it can be concluded, barring experimental error, that the putative father is not the true father.

If the set of polymorphisms in the child attributable to the father does match the set of polymorphisms of the putative father, a statistical calculation can be performed to determine the probability of coincidental match.

The probability of parentage exclusion (representing the probability that a random male will have a given polymorphic form at a given polymorphic site that makes him incompatible as the father) is given by the equation (see PCT Publication WO 95/12607):
p(exc)=xy(1−xy)
where x and y are the population frequencies of alleles A and B of a biallelic polymorphic site. (At a triallelic site p(exc)=xy(1−xy)+yz(1−yz)+xz(1−xz)+3xyz(1−xyz), where x, y and z and the respective population frequencies of alleles A, B and C).

The probability of non-exclusion is
p(non-exc)=1−p(exc)

The cumulative probability of non-exclusion (representing the value obtained when n loci are used) is thus:
cum p(non-exc)=p(non-exc1)p(non-exc2)p(non-exc3) . . . p(non-exc)

The cumulative probability of exclusion for n loci (representing the probability that a random male will be excluded)
cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

### Correlation of Polymorphisms with Phenotypic Traits

The polymorphisms of the present invention can contribute to the phenotype of an organism in different ways. Some polymorphisms occur within a protein coding sequence and contribute to phenotype by affecting protein structure. The effect can be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but can exert phenotypic effects indirectly via influence on replication, transcription, and translation. A single polymorphism can affect more than one phenotypic trait. Likewise, a single phenotypic trait can be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is or might be genetic, such as autoimmune diseases, inflammation, cancer, diseases of the nervous system, cardiovascular disease and infection by pathogenic microorganisms. Some examples of autoimmune diseases include rheumatoid arthritis, multiple sclerosis, diabetes (insulin-dependent and non- independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, decreased risk for a condition, and susceptibility or receptivity to particular drugs or therapeutic treatments.

The correlation of one or more polymorphisms with phenotypic traits can be facilitated by knowledge of the gene product of the wildtype (reference) gene. The genes in which SNPs of the present invention have been identified are genes that have been previously sequenced and characterized in one of their allelic forms. Thus, the SNPs of the invention can be used to identify correlations between one or another allelic form of the gene with a disorder with which the gene is associated, thereby identifying causative or predictive allelic forms of the gene.

Correlation can be performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of whom exhibit lack of the trait. The alleles of each polymorphism of the set are then reviewed to determine whether the presence or absence of a particular allele is associated with the trait of interest. Correlation can be performed by standard statistical methods such as a Chi-squared test and statistically significant correlations between polymorphic form(s) and phenotypic characteristics are noted. For example, it might be found that the presence of allele Al at polymorphism A correlates with heart disease. As a further example, it might be found that the combined presence of allele Al at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient might justify immediate administration of treatment, or at least the institution of regular monitoring of the patient. Detection of a polymorphic form correlated with serious disease in a couple contemplating a family might also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring might not be justified. Nevertheless, the patient can be motivated to begin simple life-style changes (e.g., diet, exercise) that can be accomplished at little cost to the patient but confer potential benefits in reducing the risk of conditions to which the patient might have increased susceptibility by virtue of variant alleles. Identification of a polymorphic set in a patient correlated with enhanced receptiveness to one of several treatment regimes for a disease indicates that this treatment regime should be followed.

### Genetic Mapping of Phenotypic Traits

Another application of the present invention comprises identification of a physical linkage between a genetic locus associated with a trait of interest and polymorphic markers that are not associated with the trait, but are in physical proximity with the genetic locus responsible for the trait and cosegregate with it. Such analysis is useful for mapping a genetic locus associated with a phenotypic trait to a chromosomal position, and thereby cloning gene(s) responsible for the trait (see, e.g., Lander et al., (1986) Proc. Natl. Acad. Sci. USA 83:7353-7357; Lander et al., (1987) Proc. Natl. Acad. Sci. USA 84:2363-2367; Donis-Keller et al., (1987) Cell 51:319-337; and Lander et al., (1989) Genetics 121:185-1999, incorporated herein by reference). Genes localized by linkage can be cloned by a process known as directional cloning (Winwright, (1993) Med. J. Australia 159:170-174; Collins, (1992) Nature Genetics 1:3-6, incorporated herein by reference).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers cosegregate with a phenotypic trait (see, e.g., Kerem et al., (1989) Science 245:1073-1080; Monaco et al., (1985) Nature 316:842; Yamoka et al., (1990) Neurology 40:222-226 (1990); Rossiter et al., (1991) FASEB J. 5:21-27).

Linkage is analyzed by calculation of LOD (log of the odds) values. A LOD value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5^{th} ed.) W.B. Saunders Company, Philadelphia, Pa., USA (1991); Strachan, in The Human Genome, BIOS Scientific Publishers Ltd, Oxford, UK, Chapter 4). A series of likelihood ratios are calculated at various recombination fractions (θ), ranging from θ=0.0 (coincident loci) to θ=0.50 (unlinked). Thus, the likelihood that a given value of θ is the ratio of the probability of data if loci linked at θ to the probability of data if loci are unlinked. The computed likelihoods are usually expressed as the log10 of this ratio (i.e., a LOD score). For example, a LOD score of 3 indicates 1000:1 odds against an apparent observed linkage being a coincidence. The use of logarithms allows data collected from different families to be combined by simple algorithm. Computer programs are available for the calculation of LOD scores for differing values of θ (e.g., LIPED, MLINK (Lathro, (1984) Proc. Nat. Acad. Sci. USA 81:3443-3446). For any particular LOD score, a recombination fraction can be determined from mathematical tables (see Smith et al., Mathematical Tables For Research Workers In Human Genetics, Churchill, London, UK, (1961); Smith, (1968) Ann. Hum. Genet. 32:127-150). The value of θ at which the LOD score is the highest is considered to be the best estimate of the recombination fraction. Positive LOD score values suggest that the two loci are linked, whereas negative values suggest that linkage is less likely (at that value of θ) than the possibility that the two loci are unlinked. By convention, a combined LOD score of +3 or greater (equivalent to greater than 1000:1 odds in favor of linkage) is considered definitive evidence that two loci are linked. Similarly, by convention, a negative LOD score of −2 or less is taken as definitive evidence against linkage of the two loci being compared. Negative linkage data are useful in excluding a chromosome or a segment thereof from consideration. The search focuses on the remaining non-excluded chromosomal locations.

### Haplotype-Based Genetic Analysis

The invention further provides methods of applying the polynucleotides and polypeptides of the present invention to the elucidation of haplotypes. Such haplotypes can be associated with any one or more of the conditions described herein (e.g., susceptibility to cardiovascular disease, etc.). A “haplotype” is defined as the pattern of a set of alleles of single nucleotide polymorphisms along a chromosome. For example, consider the case of three single nucleotide polymorphisms (SNP1, SNP2, and SNP3) in one chromosome region, of which SNP1 is an A/G polymorphism, SNP2 is a G/C polymorphism, and SNP3 is an A/C polymorphism. A and G are the alleles for the first, G and C for the second and A and C for the third SNP. Given two alleles for each SNP, there are three possible genotypes for individuals at each SNP. For example, for the first SNP, A/A, A/G and G/G are the possible genotypes for individuals. When an individual has a genotype for a SNP in which the alleles are not the same, for example A/G for the first SNP, then the individual is a heterozygote. When an individual has an A/G genotype at SNP1, G/C genotype at SNP2, and A/C genotype at SNP3, there are four possible combinations of haplotypes (A, B, C, and D) for this individual. The set of SNP genotypes of this individual alone would not provide sufficient information to resolve which combination of haplotypes this individual possesses. However, when this individual's parents' genotypes are available, haplotypes could then be assigned unambiguously. For example, if one parent had an A/A genotype at SNP1, a G/C genotype at SNP2, and an A/A genotype at SNP3, and the other parent had an A/G genotype at SNP1, C/C genotype at SNP2, and C/C genotype at SNP3, while the child was a heterozygote at all three SNPs, there is only one possible haplotype combination, assuming there was no crossing over in this region during meiosis.

When the genotype information of relatives is not available, haplotype assignment can be done using the long range-PCR method (Clark, (1990) Mol. Biol. Evol. 7(2): 111-22; Clark et al., (1998) Am. J. Hum. Genet. 63(2): 595-612; Fullerton et al., (2000) Am. J. Hum. Genet. 67(4):881-900; Templeton et al., (2000) Am. J. Hum. Genet. 66(1):69-83, incorporated herein by reference). When the genotyping result of the SNPs of interest are available from general population samples, the most likely haplotypes can also be assigned using statistical methods (Excoffier & Slatkin, (1995) Mol. Biol. Evol. 12(5):921-7; Fallin & Schork, (2000) Am. J. Hum. Genet. 67(4):947-59; Long et al., (1995) Am. J. Hum. Genet. 56(3):799-810).

Once an individual's haplotype in a certain chromosome region (i.e., locus) has been determined, it can be used as a tool for genetic association studies using different methods, which include, for example, haplotype relative risk analysis (Knapp et al., (1993) Am. J. Hum. Genet. 52(6):1085-93; Li et al., (1998) Schizophr. Res. 32(2):87-92; Matise, (1995) Genet. Epidemiol. 12(6):641-5; Ott, (1989) Genet. Epidemiol. 6(1):127-30; Terwilliger & Ott, (1992) Hum. Hered. 42(6):337-46). Haplotype based genetic analysis, using a combination of SNPs, provides increased detection sensitivity, and hence statistical significance, for genetic associations of diseases, as compared to analyses using individual SNPs as markers. Multiple SNPs present in a single gene or a continuous chromosomal region are useful for such haplotype-based analyses.

### Kits

The present invention further provides kits comprising at least one agent for identifying which alleleic form of the SNPs identified herein is present in a sample. For example, suitable kits can comprise at least one antibody specific for a particular protein or peptide encoded by one allelelic form of the gene, or allele-specific oligonucleotide as described herein. In one embodiment, a kit of the present invention comprises one or more pairs of allele-specific oligonucleotides hybridizing to different forms of a polymorphism. In another embodiment of a kit, the allele-specific oligonucleotides are provided immobilized on a substrate or support. For example, the same substrate or support can comprise allele-specific oligonucleotide probes for detecting one or more of the polymorphisms described in the Sequence Listing and/or the Figures and/or in the present disclosure. Optional additional components of the kit can include, for example, restriction enzymes, reverse-transcriptase or polymerase, substrate nucleoside triphosphates, for labeling a molecule (for example, an avidin-enzyme conjugate and enzyme substrate and chromogen if the label is biotin), and buffers for reverse transcription, PCR, or hybridization reactions. A kit can also comprise instructions for using the kit and interpreting the results of an experiment performed using the kit.

### Chromosome Identification and Mapping

In one application, the polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can thus be used as a chromosome marker.

In this application, sequences can be mapped to chromosomes by preparing PCR primers (e.g., about 15 to about 25 bp) from the sequences shown in SEQ ID NO:1, 3, 5, 7 (e.g., those shown in SEQ ID NOs:9-16). Primers can be selected using computer analysis so that primers do not span more than one predicted exon in the genomic DNA. These primers are then used for PCR screening of somatic cell hybrids containing individual human chromosomes.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as about 500 or, about 600 bases; however, polynucleotides between about 2,000 and about 4,000 bp are typically employed. For a review of this technique, see Verma et al., Human Chromosomes: a Manual of Basic Techniques, Pergamon Press, New York, N.Y., USA (1988).

For chromosome mapping, the polynucleotides can be used individually (to mark a single chromosome or a single site on that chromosome) or in panels (for marking multiple sites and/or multiple chromosomes). Representative polynucleotides correspond to the noncoding regions of the cDNAs because the coding sequences are more likely conserved within gene families, thus increasing the chance of cross hybridization during chromosomal mapping.

### Linkage Analyses

Once a polynucleotide has been mapped to a precise chromosomal location, the physical position of the polynucleotide can be used in a linkage analysis. Linkage analysis establishes coinheritance between a chromosomal location and presentation of a particular disease. Disease mapping data are known in the art. Assuming a one megabase mapping resolution and one gene per 20 kb, a cDNA precisely localized to a chromosomal region associated with the disease could be one of about 50 to abut 500 potential causative genes.

Thus, once coinheritance is established, differences in the polynucleotide and the corresponding gene between affected and unaffected organisms can be examined. First, visible structural alterations in the chromosomes, such as deletions or translocations, are examined in chromosome spreads or by PCR. If no structural alterations exist, the presence of point mutations are ascertained. A mutation observed in some or all affected organisms, but not in normal organisms, indicates that the mutation might cause the disease. However, complete sequencing of the polypeptide and the corresponding gene from several normal organisms is required to distinguish the mutation from a polymorphism. If a new polymorphism is identified, this polymorphic polypeptide can be used for further linkage analysis.

### Assessment of Gene Expression

Furthermore, increased or decreased expression of the gene in affected organisms as compared to unaffected organisms can be assessed using polynucleotides of the present invention. Any of these alterations (altered expression, chromosomal rearrangement, or mutation) can be used as a diagnostic or prognostic marker.

Thus, the invention also provides a diagnostic method useful during diagnosis of a disorder, involving measuring the expression level of polynucleotides of the present invention in cells or body fluid from an organism and comparing the measured gene expression level with a standard level of polynucleotide expression level, whereby an increase or decrease in the gene expression level compared to the standard is indicative of a disorder.

The term “measuring the expression level of a polynucleotide of the present invention” means qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). For example, the polypeptide level or mRNA level in a first biological sample is measured or estimated and compared to a standard polypeptide level or mRNA level, the standard being taken from a second biological sample obtained from an individual not having the disorder or being determined by averaging levels from a population of organisms not having a disorder. As will be appreciated by those of ordinary skill in the art, once a standard polypeptide level or mRNA level is known, it can be used repeatedly as a standard for comparison.

As used herein the term “biological sample” encompasses any biological sample obtained from an organism, body fluids, cell line, tissue culture, or other source that contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain a polypeptide of the present invention, and other tissue sources found to express a polypeptide of the present invention. Methods for obtaining tissue biopsies and body fluids from organisms are known in the art. Where the biological sample is to include mRNA, a tissue biopsy is a preferred source.

The techniques described herein can be applied in a diagnostic method and/or a kit in which polynucleotides and/or polypeptides are attached to a solid support (e.g., a multi-welled plate or plastic pins). In one exemplary method, the support can be a “gene chip” or a “biological chip” as described in U.S. Pat. Nos. 5,837,832, 5,874,219, and 5,856,174. Further, such a gene chip with polynucleotides of the present invention attached can be used to identify polymorphisms between the polynucleotide sequences, with polynucleotides isolated from a test subject. The knowledge of such polymorphisms (e.g., their location, as well as, their existence) can be beneficial in identifying disease loci for many disorders, including proliferative diseases and conditions. Such a method is described in U.S. Pat. Nos. 5,858,659 and 5,856,104.

In addition to the foregoing, a polynucleotide can be used to control gene expression through triple helix formation or antisense DNA or RNA. Antisense techniques are known (see, e.g., Okano, (1991) J. Neurochem. 56:560; Oligodeoxynucleotides as Antisense Inhibitors of Gene Expression, CRC Press, Boca Raton, Fla., USA (1988). Triple helix formation is also known, (see, e.g., Lee et al., (1979) Nucl. Acid Res. 6:3073; Cooney et al., (1988) Science 241:456; and Dervan et al., (1991) Science 251:1360). Both methods rely on binding of the polynucleotide to a complementary DNA or RNA. For these techniques, polynucleotides are usually oligonucleotides about 20 to about 40 bases in length and complementary to either the region of the gene involved in transcription (triple helix—Lee et al., (1979) Nucl. Acid Res. 6:3073; Cooney et al., (1988) Science 241:456; and Dervan et al., (1991) Science 251:1360) or to the mRNA itself (antisense—Okano, (1991) J. Neurochem. 56:560; Oligodeoxynucleotides as Antisense Inhibitors of Gene Expression, CRC Press, Boca Raton, Fla., USA (1988)). Triple helix formation optimally results in a shut-off of RNA transcription from DNA, while antisense RNA hybridization blocks translation of a mRNA molecule into polypeptide. Both techniques are effective in model systems, and the information disclosed herein can be used to design antisense or triple helix polynucleotides in an effort to treat or prevent disease.

### Peptide Nucleic Acids

The present invention also encompasses polynucleotides of the present invention that are chemically synthesized, or reproduced as peptide nucleic acids (PNA), or according to other methods known in the art. PNAs can serve as a preferred polynucleotide form if the polynucleotides are incorporated onto a solid support, or a gene chip. In the context of the present invention, a peptide nucleic acid is a polyamide type of DNA analog and the monomeric units for adenine, guanine, thymine and cytosine are available commercially (Perseptive Biosystems, Framingham, Mass., USA). Certain components of DNA, such as phosphorus, phosphorus oxides, or deoxyribose derivatives, are not present in PNAs. As disclosed by Nielsen et al. and Egholm et al., (Nielsen et al., (1991) Science 254:1497 and Egholm et al., (1993) Nature 365:666), PNAs bind specifically and tightly to complementary DNA strands and are not degraded by nucleases. In fact, PNA binds more strongly to DNA than DNA itself does. PNA/DNA duplexes bind under a wider range of stringency conditions than DNA/DNA duplexes, making it easier to perform multiplex hybridization. Smaller probes can be used than with DNA due to the stronger binding characteristics of PNA/DNA hybrids. In addition, it is more likely that single base mismatches can be determined with PNA/DNA hybridization because a single mismatch in a PNA/DNA 15-mer lowers the melting point (Tₘ) by 8°-20° C., vs. 4°-16° C. for the DNA/DNA 15-mer duplex. Also, the absence of charged groups in PNA means that efficient hybridization can be performed at low ionic strengths and reduce possible interference by salt during the analysis.

The present invention encompasses the addition of a nuclear localization signal, operably linked to the 5′ end, 3′ end, or any location therein, to any of the oligonucleotides, antisense oligonucleotides, triple helix oligonucleotides, ribozymes, PNA oligonucleotides, and/or polynucleotides, of the present invention (see, e.g., Cutrona et al., (2000) Nat. Biotech. 18:300-303).

### Gene Therapy

The polynucleotides of the present invention can also be employed in gene therapy. One goal of gene therapy is to insert a normal gene into an organism having a defective gene, in an effort to correct the genetic defect. The polynucleotides disclosed in the present invention offer an approach to targeting such genetic defects in a highly accurate manner. Another goal is to insert a new gene that was not present in the host genome, thereby producing a new trait in the host cell. In one example, a polynucleotide sequence of the present invention can be used to construct chimeric RNA/DNA oligonucleotides corresponding to the sequences, specifically designed to induce host cell mismatch repair mechanisms in an organism upon systemic injection, for example (Bartlett et al., (2000) Nat. Biotech. 18:615-622). Such RNA/DNA oligonucleotides can be designed to correct genetic defects in certain host strains, and/or to introduce desired phenotypes in the host (e.g., introduction of a specific polymorphism within an endogenous gene corresponding to a polynucleotide of the present invention that can ameliorate and/or prevent a disease symptom and/or disorder, etc.). Alternatively, a polynucleotide sequence of the present invention can be used to construct duplex oligonucleotides corresponding to the sequence, specifically designed to correct genetic defects in certain host strains, and/or to introduce desired phenotypes into the host (e.g., introduction of a specific polymorphism within an endogenous gene corresponding to a polynucleotide of the present invention that can ameliorate and/or prevent a disease symptom and/or disorder, etc). Such methods of using duplex oligonucleotides are known in the art and are encompassed by the present invention (see, e.g., EP 1007712).

One aspect of the present invention is directed to gene therapy methods for treating or preventing disorders, diseases and conditions. The gene therapy methods relate to the introduction of nucleic acid (DNA, RNA and antisense DNA or RNA) sequences into an animal to achieve expression of a polypeptide of the present invention. This method requires a polynucleotide that codes for a polypeptide of the invention is operatively linked to a promoter and any other genetic elements necessary for the expression of the polypeptide by the target tissue. Such gene therapy and delivery techniques are known in the art, see, for example, PCT Publication WO 90/11092.

Thus, for example, cells from a patient can be engineered with a polynucleotide (DNA or RNA) comprising a promoter operably linked to a polynucleotide of the invention ex vivo, with the engineered cells then being provided to a patient to be treated with the polypeptide. Such methods are known in the art. For example, see Belldegrun et al., (1993) J. Natl. Cancer Inst. 85:207-216; Ferrantini et al., (1993) Cancer Res. 53:107-1112; Ferrantini et al., (1994) J. Immunol. 153: 604-4615; Kaido et al., (1995) Int. J. Cancer 60:221-229; Ogura et al., (1990) Cancer Res. 50: 5102-5106; Santodonato et al., (1996) Human Gene Therapy 7:1-10; Santodonato et al., (1997) Gene Therapy 4:1246-1255; and Zhang et al., (1996) Cancer Gene Therapy 3:31-38). In one embodiment, the cells that are engineered are arterial cells. The arterial cells can be reintroduced into the patient through direct injection to the artery, the tissues surrounding the artery, or through catheter injection.

As described herein, a polynucleotide construct can be delivered by any method that delivers injectable materials to the cells of an animal, such as, injection into the interstitial space of tissues (heart, muscle, skin, lung, liver, and the like). A polynucleotide construct can be delivered in a pharmaceutically acceptable liquid or aqueous carrier.

In one embodiment, a polynucleotide of the present invention is delivered as a naked polynucleotide. The term “naked polynucleotide” refers to a DNA or RNA sequence that is free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859.

A polynucleotide vector construct of the present invention used in the gene therapy method can comprise a construct that will not integrate into the host genome nor will it comprise a sequence that allows for replication. Representative vectors include pWLNEO, pSV2CAT, pOG44, pXT1 and pSG available from Stratagene, La Jolla, Calif., USA; pSVK3, pBPV, pMSG and pSVL available from Pharmacia, Piscataway, N.J., USA; and pEF1JV5, pcDNA3.1, and pRc/CMV2 available from Invitrogen, Carlsbad, Calif., USA. Other suitable vectors will be readily apparent to those of ordinary skill in the art upon consideration of the present disclosure.

Any strong promoter known to those skilled in the art can be used for driving the expression of a polynucleotide sequence of the present invention as a component of a gene therapy method. Representative promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter can also be the native promoter for the polynucleotides of the present invention.

Unlike other gene therapy techniques, one major advantage of introducing a naked nucleic acid sequence into a target cell is the transitory nature of the polynucleotide synthesis in the cells. Studies have shown that non-replicating DNA sequences can be introduced into cells to provide production of a desired polypeptide for periods of up to six months.

A polynucleotide construct of the present invention can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. The interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. Similarly, the term also refers to the space occupied by the plasma of the circulation and the lymph fluid of the lymphatic channels.

In some embodiments, delivery of a polynucleotide of the present invention to the interstitial space of muscle tissue can be desirable. A polynucleotide of the present invention can be conveniently delivered by injection into such tissues. Delivery to, and expression of a polynucleotide of the present invention in, muscle and other tissues comprising persistent, non-dividing cells which are differentiated can be desirable; delivery and expression can, however, be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA can be, for example, in the range of from about 0.05 mg/kg body weight to about 50 mg/kg body weight. In another example, the dosage can be from about 0.005 mg/kg to about 20 mg/kg and in yet another example, from about 0.05 mg/kg to about 5 mg/kg. Of course, as one of ordinary skill in the art will appreciate, this dosage will vary according to the tissue site of injection. The appropriate and effective dosage of nucleic acid sequence can readily be determined by one of ordinary skill in the art and can depend on the condition being treated and the route of administration.

One representative route of administration is via the parenteral route of injection into the interstitial space of tissues. Other parenteral routes can also be used, however, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns.” These delivery methods are known in the art.

The constructs can also be delivered via delivery vehicles such as viral sequences, viral particles, liposome formulations, lipofectin, precipitating agents, etc. Such methods of delivery are known in the art and the applicability of these methods will be known to those of ordinary skill in the art, upon consideration of the present disclosure.

In some embodiments, a polynucleotide construct of the present invention can be complexed in a liposome preparation. Liposomal preparations for use in the instant invention include cationic (positively charged), anionic (negatively charged) and neutral preparations. Sometimes, however, cationic liposomes are preferred because a tight charge complex can be formed between the cationic liposome and the polyanionic nucleic acid. Cationic liposomes have been shown to mediate intracellular delivery of plasmid DNA (Felgner et al., (1987) Proc. Natl. Acad. Sci. USA 84:7413-7416); mRNA (Malone et al., (1989) Proc. Natl. Acad. Sci. USA 86:6077-6081); and purified transcription factors (Debs et al., (1990) J. Biol. Chem. 265:10189-10192), in functional form.

Cationic liposomes are commercially available. For example, N[1-2,3-dioleyloxy)propyl]-N,N,N-triethylammonium (DOTMA) liposomes are particularly useful and are available under the trademark LIPOFECTIN®, from GIBCO BRL, Grand Island, N.Y., USA (see, also, Felgner et al., (1987) Proc. Natl. Acad. Sci. USA 84:7413-7416). Other commercially available liposomes include transfectace (DDAB/DOPE) and DOTAP/DOPE (Boehringer).

Other cationic liposomes can be prepared from readily available materials using techniques well known in the art. See, e.g. PCT Publication WO 90/11092 for a description of the synthesis of DOTAP (1,2-bis (oleoyloxy)-3-(trimethylammonio)propane) liposomes. Preparation of DOTMA liposomes is also described in the literature, see, e.g., Felgner et al., (1987) Proc. Natl. Acad. Sci. USA 84:7413-7417. Similar methods can be used to prepare liposomes from other cationic lipid materials.

Similarly, anionic and neutral liposomes are readily available, such as from Avanti Polar Lipids (Birmingham, Ala., USA), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator (Heat Systems, Farmingdale, N.Y., USA) equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., (1983) Methods Immunol. 101:512-527. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments.

LUVs are prepared by a number of methods, well known in the art. Commonly used methods include Ca²⁺-EDTA chelation (Papahadjopoulos et al., (1975) Biochim. Biophys. Acta 394:483; Wilson et al., (1979) Cell 17:77); ether injection (Deamer et al., (1976) Biochim. Biophys. Acta 443:629; Ostro et al., (1977) Biochem. Biophys. Res. Commun. 76:836; Fraley et al., (1979) Proc. Natl. Acad. Sci. USA 76:3348); detergent dialysis (Enoch et al., (1979) Proc. Natl. Acad. Sci. USA 76:145); and reverse-phase evaporation (REV) (Fraley et al., (1980) J. Biol. Chem. 255:10431; Szoka et al., (1978) Proc. Natl. Acad. Sci. USA 75:145; Schaefer-Ridder et al., (1982) Science 215:166.

Generally, the ratio of DNA to liposomes will be from about 10:1 to about 1:10. Preferably, the ration will be from about 5:1 to about 1:5. More preferably, the ratio will be about 3:1 to about 1:3. Still more preferably, the ratio will be about 1:1.

U.S. Pat. No. 5,676,954 describe the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and PCT Publication WO 94/9469 describe cationic lipids for use in transfecting DNA into cells and mammals. U.S. Pat. Nos. 5,589,466, 5,693,622, 5,580,859, 5,703,055, and PCT Publication WO 94/9469 provide methods for delivering DNA-cationic lipid complexes to mammals.

In certain embodiments, cells are engineered, ex vivo or in vivo, using a retroviral particle containing RNA that comprises a sequence encoding polypeptides of the invention. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

A retroviral plasmid vector is typically employed to transduce packaging cell lines to form producer cell lines. Examples of packaging cells that can be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, (1990) Human Gene Therapy 1:5-14. The vector can transduce the packaging cells through any means known in the art. Such means include, but are not limited to, electroporation, the use of liposomes, and CaPO₄ precipitation. In one alternative, the retroviral plasmid vector can be encapsulated into a liposome, or coupled to a lipid, and then administered to a host.

The producer cell line generates infectious retroviral vector particles that include polynucleotide encoding polypeptides of the invention. Such retroviral vector particles can then be employed, to transduce eukaryotic cells, either in vitro or in vivo. The transduced eukaryotic cells will then express polypeptides of the present invention.

In certain other embodiments, cells are engineered, ex vivo or in vivo, with polynucleotides of the invention contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz et al., (1974) Am. Rev. Respir. Dis. 109:233-238). Additionally, adenovirus mediated gene transfer has been demonstrated in a number of instances including transfer of alpha-1-antitrypsin and CFTR to the lungs of cotton rats (Rosenfeld et al., (1991) Science 252:431-434; Rosenfeld et al., (1992) Cell 68:143-155). Furthermore, extensive studies to attempt to establish adenovirus as a causative agent in human cancer were uniformly negative (Green et al., (1979) Proc. Natl. Acad. Sci. USA 76:6606).

Suitable adenoviral vectors useful in the present invention are described, for example, in Kozarsky & Wilson, (1993) Curr. Opin. Genet. Devel. 3:499-503; Rosenfeld et al., (1992) Cell 68:143-155; Engelhardt et al., (1993) Human Genet. Ther. 4:759-769; Yang et al., (1994) Nature Genet. 7:362-369; Wilson et al., (1993) Nature 365:691-692; and U.S. Pat. No: 5,652,224. For example, the adenovirus vector Ad2 is useful and can be grown in human embryonic kidney cells (HEK293 cells). These cells contain the El region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

In one embodiment, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses can be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, (1992) Curr. Topics Microbiol. Immunol. 158:97). It is also one of the few viruses that can integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, (3^{rd} ed.) Cold Spring Harbor Laboratory Press, Cold Spring Harbor, N.Y., USA (2001). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles that contain the polynucleotide construct of the invention. These viral particles are then used to transduce eukaryotic cells, either ex vivo or in vivo. The transduced cells will contain the polynucleotide construct integrated into its genome, and will express the desired gene product.

Another method of gene therapy involves operably associating heterologous control regions and endogenous polynucleotide sequences (e.g. encoding the polypeptide sequence of interest) via homologous recombination (see, e.g., U.S. Pat. No. 5,641,670; PCT Publications WO 96/29411 and WO 94/12650; Koller et al., (1989) Proc. Natl. Acad. Sci. USA 86:8932-8935; and Zijlstra et al., (1989) Nature 342:435-438. This method involves the activation of a gene which is present in the target cells, but which is not normally expressed in the cells, or is expressed at a lower level than desired.

Polynucleotide constructs are made, using standard techniques known in the art, that contain the promoter with targeting sequences flanking the promoter. Suitable promoters are described herein and are known in the art. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. In one arrangement, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. Such methods are described in more detail herein.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotides encoding polypeptides of the present invention can be administered along with other polynucleotides encoding plasma HDL level-modulating proteins.

In one embodiment, the polynucleotide encoding a polypeptide of the invention contains a secretory signal sequence that facilitates secretion of the protein. Typically, the signal sequence is positioned in the coding region of the polynucleotide to be expressed towards or at the 5′ end of the coding region. The signal sequence can be homologous or heterologous to the polynucleotide of interest and can be homologous or heterologous to the cells to be transfected. Additionally, the signal sequence can be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., (1989) Science 243:375).

A desirable method of local administration is by direct injection. For example, a recombinant molecule of the present invention complexed with a delivery vehicle is administered by direct injection into or locally within the area of arteries. Administration of a composition locally within the area of arteries refers to injecting the composition within centimeters and preferably, millimeters of an artery.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration include recombinant molecules of the present invention complexed to a targeted delivery vehicle of the present invention. Suitable delivery vehicles for use with systemic administration comprise liposomes comprising ligands for targeting the vehicle to a particular site.

Representative methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., (1992) Proc. Natl. Acad. Sci. USA 189:11277-11281). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, for example to mammals and birds. Representative mammals include humans, dogs, cats, mice, rats, rabbits sheep, cattle, horses, pigs, and particularly humans.

### Identification of an Organism and/or Tissue Type

The polynucleotides of the present invention are also useful for identifying organisms in minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an organism's genome. These sequences can be used to prepare PCR primers for amplifying and isolating such selected DNA, which can then be sequenced. Using this technique, organisms can be identified because each organism will have a unique set of DNA sequences. Once a unique ID database is established for an organism, positive identification of that organism, living or dead, can be made from extremely small samples. Similarly, polynucleotides of the present invention can be used as polymorphic markers, in addition to, the identification of transformed or non-transformed cells and/or tissues.

There is also a need for reagents capable of identifying the source of a particular tissue. Such need arises, for example, when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination. Moreover, as mentioned above, such reagents can be used to screen and/or identify transformed and non-transformed cells and/or tissues.

Thus, the present invention comprises a method of identifying the species, tissue or cell type of a biological sample, the method comprising detecting a nucleic acid molecule in the sample, if any, comprising a nucleotide sequence containing one or more polymorphic positions and corresponding to, or derived from, a sequence selected from the group consisting of: a nucleotide sequence of SEQ ID NOs:1, 3, 5, and 7, and fragments thereof.

Another representative embodiment comprises a method of identifying the species, tissue or cell type of a biological sample comprising detecting polypeptide molecules in the sample, if any, comprising an amino acid sequence comprising one or more polymorphic positions and is derived from, or corresponds to, a sequence selected from the group consisting of an amino acid sequence of SEQ ID NOs:2, 4, 6 and 8.

In any of the methods of the present invention, the step of detecting a polypeptide molecule can comprise using an antibody.

The method for identifying the species, tissue or cell type of a biological sample can comprise a step of detecting nucleic acid molecules comprising a nucleotide sequence in a panel of at least two nucleotide sequences, wherein at least one sequence in the panel contains one or more polymorphic positions to a sequence selected from the group.

In yet another application, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

### Representative Applications of the Polypeptides of the Present Invention

A polypeptide of the present invention can be employed in a range of applications. The following descriptions are exemplary and non-limiting; techniques for carrying out the following applications are known in the art and will be apparent to those of ordinary skill in the art upon consideration of the present disclosure and the novel nucleotide and polypeptide sequences described herein. Additional applications for the polypeptides of the present invention will become apparent to those of ordinary skill in the art upon consideration of the present disclosure.

As described herein, a polypeptide of the present invention can be produced recombinantly. Thus, in one embodiment the present invention comprises a method of making an isolated polypeptide comprising culturing a recombinant host cell of the present invention under conditions such that the polypeptide is expressed and recovering the polypeptide. Another representative embodiment comprises this method of making an isolated polypeptide, wherein the recombinant host cell is a eukaryotic cell and the polypeptide is a protein comprising an amino acid sequence selected from the group consisting of: an amino acid sequence of SEQ ID NOs:2, 4, 6 and 8, and fragments thereof. The isolated polypeptide produced by this method is also an aspect of the present invention.

In one embodiment the present invention comprises a method for detecting, in a biological sample comprising a polypeptide, a polypeptide comprising an amino acid sequence comprising one or more polymorphic positions and is derived from, or corresponds to, a sequence selected from the group consisting of: an amino acid sequence of SEQ ID NOs:2, 4, 6 and 8, and fragments thereof, the method comprising comparing an amino acid sequence of at least one polypeptide molecule in the sample with a sequence selected from the group and determining whether the sequence of said polypeptide molecule in said sample containing one or more polymorphic positions and is derived from, or corresponds to a sequence of the group.

In another embodiment the present invention comprises the above method wherein the comparing comprises determining the extent of specific binding of a polypeptide in the sample to an antibody that binds specifically to a polypeptide comprising an amino acid sequence containing one or more polymorphic positions and is derived from, or corresponds to, a sequence selected from the group consisting of: an amino acid sequence of SEQ ID NOs:2, 4, 6 and 8, and fragments thereof.

Polypeptides of the present invention can also be used to treat, prevent, and/or diagnose disease. For example, patients can be administered a polypeptide of the present invention in an effort to replace absent or decreased levels of the polypeptide (e.g., SREBP1), to supplement absent or decreased levels of a different polypeptide (e.g., hemoglobin S for hemoglobin B, SOD, catalase, DNA repair proteins), to inhibit the activity of a polypeptide (e.g., an oncogene or tumor suppressor), to activate the activity of a polypeptide (e.g., by binding to a receptor), to reduce the activity of a membrane bound receptor by competing with it for free ligand, or to bring about a desired response (e.g., an increase in plasma HDL levels or a decrease in conditions related to a cardiovascular disorder).

Antibodies directed to a polypeptide of the present invention can also be used to treat, prevent, and/or diagnose disease. For example, administration of an antibody directed to a-polypeptide of the present invention can bind and reduce overproduction of the polypeptide. Similarly, administration of an antibody can activate the polypeptide, such as by binding to a polypeptide bound to a membrane (or a membrane-bound receptor).

Further, a polypeptide of the present invention can be used as molecular weight markers on SDS-PAGE gels or on molecular sieve gel filtration columns using methods known to those of skill in the art. Polypeptides can also be used to raise antibodies, which in turn are used to measure protein expression from a recombinant cell, as a way of assessing transformation of the host cell.

A polypeptide of the present invention can also be used to screen for molecules that bind to the polypeptide, or for molecules to which the polypeptide binds. The binding of the polypeptide and the molecule can activate (agonist), increase, inhibit (antagonist), or decrease activity of the polypeptide or the molecule bound. Examples of such molecules include antibodies, oligonucleotides, proteins (e.g., receptors), or small molecules.

In some cases, it is desirable that, the molecule is closely related to a natural ligand of the polypeptide, e.g., a fragment of the ligand, or a natural substrate, a ligand, a structural or functional mimetic (see, Coligan et al., (1991) Current Protocols in Immunology 1(2):Chapter 5). Similarly, the molecule can be closely related to the natural receptor to which the polypeptide binds, or at least, a fragment of the receptor capable of being bound by the polypeptide (e.g., active site). In either case, the molecule can be rationally designed using known techniques.

Screening for these molecules can involve producing cells that express the polypeptide, either as a secreted protein or on the cell membrane. Representative cells include cells from mammals, yeast, Drosophila, or E. coli. Cells expressing the polypeptide (or cell membrane containing the expressed polypeptide) are then contacted with a test compound potentially containing the molecule to observe binding, stimulation, or inhibition of activity of either the polypeptide or the molecule.

The assay can simply test binding of a candidate compound to the polypeptide, wherein binding is detected by a label, or in an assay involving competition with a labeled competitor. Further, the assay can test whether the candidate compound results in a signal generated by binding to the polypeptide.

Alternatively, the assay can be carried out using cell-free preparations, polypeptide/molecule affixed to a solid support, chemical libraries, or natural product mixtures. The assay can also simply comprise the steps of mixing a candidate compound with a solution containing a polypeptide, measuring polypeptide/molecule activity or binding, and comparing the polypeptide/molecule activity or binding to a standard.

In one example, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

Additionally, the receptor to which a polypeptide of the invention binds can be identified by numerous methods known to those of skill in the art, for example, ligand panning and FACS sorting (Coligan et al., (1991) Current Protocols in Immunology 1(2):Chapter 5). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labeled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clone that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) can be employed to modulate the activities of polypeptides of the invention thereby effectively generating agonists and antagonists of polypeptides of the invention. See generally, U.S. Pat. Nos. 5,605,793, 5,811,238, 5,830,721, 5,834,252, and 5,837,458, and Patten et al., (1997) Curr. Opinion Biotechnol. 8:724-33; Harayama, (1998) Trends Biotechnol. 16 (2):76-82; Hansson et al., (1999) J. Mol. Biol. 287:265-76; and Lorenzo & Blasco, (1998) Biotechniques 24(2):308-13. In one embodiment, alteration of polynucleotides and corresponding polypeptides of the invention can be achieved by DNA shuffling. DNA shuffling involves the assembly of two or more DNA segments into a desired polynucleotide sequence of the invention molecule by homologous, or site-specific, recombination. In another embodiment, polynucleotides and corresponding polypeptides of the invention can be altered by being subjected to random mutagenesis by error-prone PCR, random nucleotide insertion or other methods prior to recombination. In another embodiment, one or more components, motifs, sections, parts, domains, fragments, etc., of the polypeptides of the invention can be recombined with one or more components, motifs, sections, parts, domains, fragments, etc. of one or more heterologous molecules. In some representative embodiments, the heterologous molecules are family members. In further representative embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic (dpp), 60A, OP-2, dorsalin, growth differentiation factors (GDFs), nodal, MIS, inhibin-alpha, TGF-beta1, TGF-beta2, TGF-beta3, TGF-beta5, and glial-derived neurotrophic factor (GDNF).

Other preferred fragments are biologically active fragments of the polypeptides of the invention. Biologically active fragments are those exhibiting activity similar, but not necessarily identical, to an activity of the polypeptide. The biological activity of the fragments can include an improved desired activity, or a decreased abnormal activity.

Additionally, the present invention provides a method of screening compounds to identify those that modulate the action of the polypeptide of the present invention. An example of such an assay comprises combining a mammalian fibroblast cell, a polypeptide of the present invention, a compound to be screened and ³[H] thymidine under cell culture conditions where the fibroblast cell would normally proliferate. A control assay can be performed in the absence of the compound to be screened and compared to the amount of fibroblast proliferation in the presence of the compound to determine if the compound stimulates proliferation by determining the uptake of ³[H] thymidine in each case. The amount of fibroblast cell proliferation is measured by liquid scintillation chromatography, which measures the incorporation of ³[H] thymidine. Both agonist and antagonist compounds can be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat, prevent, and/or diagnose disease or to bring about a particular result in a patient (e.g., blood vessel growth) by activating or inhibiting the polypeptide/molecule. Moreover, the assays can discover agents that might inhibit or enhance the production of the polypeptides of the invention from suitably manipulated cells or tissues. Therefore, the invention includes a method of identifying compounds that bind to a polypeptide of the present invention comprising the steps of: (a) incubating a candidate binding compound with a polypeptide of the present invention; and (b) determining if binding has occurred. Moreover, the invention includes a method of identifying agonists/antagonists comprising the steps of: (a) incubating a candidate compound with the polypeptide, (b) assaying a biological activity, and (b) determining if a biological activity of the polypeptide has been altered.

Another embodiment of the present invention comprises an isolated polypeptide comprising an amino acid sequence containing one or more polymorphic positions and is derived from, or corresponds to an amino acid sequence selected from the group consisting of SEQ ID NO:2, 4, 6 and 8, and fragments thereof.

### Diagnosis and Treatment of Diseases and Conditions

In one aspect, the polynucleotides, polypeptides, agonists and/or antagonists of the invention can be used to treat, prevent, and/or diagnose cardiovascular diseases, disorders, and/or conditions, including conditions associated with undesirable and/or unhealthy plasma HDL levels (e.g., less than about 40 mg/dL of plasma). The polynucleotides, polypeptides, agonists and/or antagonists of the invention can also be used to enhance plasma HDL levels and/or diagnose, detect or predict elevated plasma HDL levels.

Cardiovascular diseases, disorders, and/or conditions include cardiovascular abnormalities, such as arterio-arterial fistula, arteriovenous fistula, cerebral arteriovenous malformations, congenital heart defects, pulmonary atresia, and Scimitar Syndrome.

Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, cardiovascular syphilis, and cardiovascular tuberculosis.

Cardiovascular diseases further include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Polynucleotides or polypeptides, or agonists or antagonists of the invention, are especially effective for predicting, treating or decreasing the risk of cardiovascular disease and/or elevating plasma HDL levels.

Polypeptides can be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Polypeptides of the invention can be administered as part of a Therapeutic, described in more detail herein. Additional methods of delivering polynucleotides of the invention follow.

In another embodiment, the present invention provides a method of delivering compositions to targeted cells expressing a receptor for a polypeptide of the present invention, or cells expressing a cell bound form of a polypeptide of the invention.

As discussed herein, polypeptides or antibodies of the invention can be associated with heterologous polypeptides, heterologous nucleic acids, toxins, or prodrugs via hydrophobic, hydrophilic, ionic and/or covalent interactions.

In one embodiment, the present invention provides a method for the specific delivery of compositions of the invention to cells by administering polypeptides of the present invention (including antibodies) that are associated with heterologous polypeptides or nucleic acids. In one example, the present invention provides a method for delivering a therapeutic protein into the targeted cell. In another example, the present invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In yet another embodiment, the present invention provides a method for the specific destruction of cells (e.g., the destruction of tumor cells) by administering polypeptides of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that can be used according to the methods of the present invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that can be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

In specific embodiments, antagonists according to the present invention comprise nucleic acids corresponding to the sequences contained in a sequence selected from the group consisting of SEQ ID NOs:1, 3, 5, and 7, and complementary strands thereof. In one embodiment, an antisense sequence is generated internally by the organism, and in another embodiment, an antisense sequence is separately administered (see, e.g., O'Connor, (1991) Neurochem. 56:560; Oligodeoxynucleotides as Antisense Inhibitors of Gene Expression, CRC Press, Boca Raton, Fla., USA (1988)). Antisense technology can be used to control gene expression through antisense DNA or RNA, or through triple-helix formation. Antisense techniques are discussed for example, in Okano, (1991) Neurochem. 56:560 and in Oligodeoxynucleotides as Antisense Inhibitors of Gene Expression, CRC Press, Boca Raton, Fla., USA (1988). Triple helix formation is discussed in, for instance, Lee et al., (1979) Nucl. Acid Res. 6:3073; Cooney et al., (1988) Science 241:456 and Dervan et al., (1991) Science 251:1300. The methods are based on binding of a polynucleotide to a complementary DNA or RNA.

For example, the use of c-myc and c-myb antisense RNA constructs to inhibit the growth of the non-lymphocytic leukemia cell line HL-60 and other cell lines has been previously described. These experiments were performed in vitro by incubating cells with the oligoribonucleotide. A similar procedure for in vivo use is described in PCT Publication WO 91/15580. Briefly, a pair of oligonucleotides for a given antisense RNA is produced as follows: a sequence complimentary to the first 15 bases of the open reading frame is flanked by an EcoR1 site on the 5′ end and a Hindi site on the 3′ end. Next, the pair of oligonucleotides is heated at 90° C. for one minute and then annealed in 2× ligation buffer (20 mM TRIS HCl pH 7.5, 10 mM MgCl₂, 10 mM dithiothreitol (DTT) and 0.2 mM ATP) and then ligated to the EcoR1/Hind Im site of the retroviral vector PMV7 (see PCT Publication WO 91/15580).

For example, the 5′ coding portion of a polynucleotide that encodes a mature polypeptide of the present invention can be used to design an antisense RNA oligonucleotide of from about 10 to about 40 base pairs in length. A DNA oligonucleotide is designed to be complementary to a region of the gene involved in transcription thereby preventing transcription and the production of the polypeptide. The antisense RNA oligonucleotide hybridizes to the mRNA in vivo and blocks translation of the mRNA molecule into polypeptide.

In one embodiment, an antisense nucleic acid of the present invention is produced intracellularly by transcription from an exogenous sequence. For example, a vector or a portion thereof, is transcribed, producing an antisense nucleic acid (RNA) of the invention. Such a vector can contain a sequence encoding the antisense nucleic acid of the invention. Such a vector can remain episomal or become chromosomally integrated, as long as it can be transcribed to produce the desired antisense RNA. Such vectors can be constructed by recombinant DNA technology methods known in the art. Vectors can be plasmid, viral, or others known in the art, used for replication and expression in vertebrate cells. Expression of a nucleotide sequence encoding a polypeptide of the present invention, or fragments thereof, can be by any promoter known in the art to act in vertebrate, for example human cells. Such promoters can be inducible or constitutive. Such promoters include, but are not limited to, the SV40 early promoter region (Bernoist & Chambon, (1981) Nature 29:304-310), the promoter contained in the 3′ long terminal repeat of Rous sarcoma virus (Yamamoto et al., (1980) Cell 22:787-797, the herpes thymidine promoter (Wagner et al., (1981) Proc. Natl. Acad. Sci. U.S.A. 78:1441-1445, the regulatory sequences of the metallothionein gene (Brinster et al., (1982) Nature 296:39-42), etc.

An antisense nucleic acid of the present invention can comprise a sequence complementary to at least a portion of an RNA transcript of a gene of interest. However, absolute complementarity, although often desirable, is not required. A sequence “complementary to at least a portion of an RNA,” as used herein, means a sequence having sufficient complementarity to be able to hybridize with the RNA, forming a stable duplex; in the case of double stranded antisense nucleic acids of the invention, a single strand of the duplex DNA can thus be tested, or triplex formation can be assayed. The ability to hybridize can depend on both the degree of complementarity and the length of the antisense nucleic acid. Generally, the larger the hybridizing nucleic acid, the more base mismatches with a RNA sequence of the present invention it can contain and still form a stable duplex (or triplex, as the case may be). One of ordinary skill in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, are expected to work most efficiently at inhibiting translation. However, sequences complementary to the 3′ untranslated sequences of mRNAs have also been shown to be effective at inhibiting translation of mRNAs as well. See generally, Wagner, (1994) Nature 372:333-335. Thus, oligonucleotides complementary to either the 5′- or 3′-non-translated, non-coding regions of a polynucleotide sequence of the invention can be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of mRNA, antisense nucleic acids are preferably at least six nucleotides in length, for example oligonucleotides ranging from about 6 to about 50 nucleotides in length. In specific aspects the oligonucleotide is at least 10 nucleotides, at least 17 nucleotides, at least 25 nucleotides or at least 50 nucleotides.

The polynucleotides of the present invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., (1989) Proc. Natl. Acad. Sci. U.S.A. 86:6553-6556; Lemaitre et al., (1987) Proc. Natl. Acad. Sci., U.S.A. 84:648-652; PCT Publication WO 88/09810) or the blood-brain barrier (see, e.g., PCT Publication WO 89/10134), hybridization-triggered cleavage agents. (see, e.g., Krol et al., (1988) BioTechniques 6:958-976) or intercalating agents. (see, e.g., Zon, (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

An antisense oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

An antisense oligonucleotide can also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., (1987) Nucl. Acids Res. 15:6625-6641). The oligonucleotide is a 2-0-methylribonucleotide (Inoue et al., (1987) Nucl. Acids Res. 15:6131-6148), or a chimeric RNA-DNA analogue (Inoue et al., (1987) FEBS Lett. 215:327-330).

As noted herein, polynucleotides of the present invention can be synthesized by standard methods known in the art, e.g. by use of an automated DNA synthesizer (such equipment is commercially available from Applied Biosystems of Foster City Calif., USA, etc.). As examples, phosphorothioate oligonucleotides can be synthesized via the method of Stein et al. (Stein et al., (1988) Nucl. Acids Res. 16:3209), methylphosphonate oligonucleotides can be prepared by use of controlled pore glass polymer supports (Sarin et al., (1988) Proc. Natl. Acad. Sci. U.S.A. 85:7448-7451), etc.

While antisense nucleotides complementary to the coding region sequence of the invention can be used, those complementary to the transcribed untranslated region are often most desirable.

Potential antagonists according to the invention also include catalytic RNA, or a ribozyme (see, e.g., PCT Publication WO 90/11364; Sarver et al., (1990) Science 247:1222-1225). While ribozymes that cleave mRNA at site specific recognition sequences can be used to destroy mRNAs corresponding to the polynucleotides of the invention, the use of hammerhead ribozymes is often preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is known in the art and is described more fully in Haseloff & Gerlach, (1988) Nature 334:585-591. There are numerous potential hammerhead ribozyme cleavage sites within each nucleotide sequence disclosed in the Figures and/or in the Sequence Listing. In one embodiment, the ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

As in an antisense approach, a ribozyme of the invention can comprise modified oligonucleotides (e.g. for improved stability, targeting, etc.) and can be delivered to cells that express the polynucleotides of the invention in vivo. DNA constructs encoding the ribozyme can be introduced into the cell in the same manner as described above for the introduction of antisense encoding DNA. A representative method of delivery involves using a DNA construct “encoding” the ribozyme under the control of a strong constitutive promoter, such as, for example, pol III or pol II promoter, so that transfected cells will produce sufficient quantities of the ribozyme to destroy endogenous messages and inhibit translation. Since ribozymes, unlike antisense molecules, are catalytic, a lower intracellular concentration is required for efficiency.

An antagonist/agonist compound can be employed to modulate plasma HDL levels. An antagonist/agonist can also generally be employed to treat, prevent, and/or diagnose the diseases described herein, notably cardiovascular diseases.

Thus, the present invention provides a method of treating or preventing diseases, disorders, and/or conditions, including but not limited to the diseases, disorders and/or conditions listed herein, associated with overexpression of a polynucleotide of the present invention by administering to a patient (a) an antisense molecule directed to a polynucleotide of the present invention, and/or (b) a ribozyme directed to a polynucleotide of the present invention.

The present invention also comprises a method of diagnosing in a subject a condition associated with SREBP1, the method comprising detecting in a biological sample obtained from a subject a nucleic acid molecule, if any, comprising a nucleotide sequence comprising one or more polymorphic positions and corresponds to, or is derived from, a sequence selected from the group consisting of: a nucleotide sequence of SEQ ID NOs:1, 3, 5, and 7, and fragments thereof.

The method for diagnosing a condition can comprise detecting nucleic acid molecules comprising a nucleotide sequence in a panel of at least two nucleotide sequences, wherein at least one sequence in said panel comprises one or more polymorphic positions and is derived from, or corresponds to a sequence selected from the group.

Another representative embodiment of the present invention comprises a method of treating of an individual in need of an increased level of a protein activity comprising administering to the individual a pharmaceutical composition comprising an amount of an isolated polypeptide, polynucleotide, or antibody of the present invention effective to increase the level of said protein activity in said individual.

Yet another representative embodiment comprises a method of treating of an individual in need of a decreased level of a protein activity comprising administering to the individual a pharmaceutical composition comprising an amount of an isolated polypeptide, polynucleotide, or antibody of the present invention effective to increase the level of said protein activity in said individual.

### EXAMPLES

The following Examples have been included to illustrate various exemplary modes of the invention. Certain aspects of the following Examples are described in terms of techniques and procedures found or contemplated by the inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

### Association of SNP with Phenotype

In a study of African-American patients enrolled in a Cholesterol and Recurrent Events trial, an association was found between two polymorphisms that result in methionine-for-valine amino acid substitutions at codons 417 and 580 in the Sterol Response Element Binding Protein (SREBP1) gene and plasma HDL levels. The results are summarized in Table 3 below:

TABLE 3
SREBP1 genotype

| | | | Met417/ | |
|---|---|---|---|---|
| Trait | Val417/Val417 | Val417/Met417 | Met417 | P value |
| Plasma HDL, | 41.4 ± 9.4 | 52.7 ± 15.0 | 34.5 ± 4.9 | 0.006 |
| mg/dL | | | | |
| (Mean ± SD) | | | | |
| | | | Met580/ | |
| | Val580/Val580 | Val580/Met580 | Met/580 | |
| Plasma HDL, | 34.2 ± 5.8 | 46.3 ± 5.3 | None | 0.006 |
| mg/dL | | | Available | |
| (Mean ± SD) | | | | |

The sequences of SREBP1 cDNAs coding for the identified reference and variant alleles are given below (the polymorphic nucleotides are indicated in bold capitals). A Met417, Val417 SREB cDNA may have the sequence indicated:

| | |
|---|---|
| SREBP1 Val417, Val480 (Reference) (SEQ ID NO:1) | |
| taacgaggaa cttttcgccg gcgccgggcc gcctctgagg ccagggcagg acacgaacgc | 61 |
| gcggagcggc ggcggcgact gagagccggg gccgcggcgg cgctccctag gaagggccgt | 121 |
| acgaggcggc gggcccggcg ggcctcccgg aggaggcggc tgcgccatgg acgagccacc | 181 |
| cttcagcgag gcggctttgg agcaggcgct gggcgagccg tgcgatctgg acgcggcgct | 241 |
| gctgaccgac atcgaagaca tgcttcagct tatcaacaac caagacagtg acttccctgg | 301 |
| cctatttgac ccaccctatg ctgggagtgg ggcagggggc acagaccctg ccagccccga | 361 |
| taccagctcc ccaggcagct tgtctccacc tcctgccaca ttgagctcct ctcttgaagc | 421 |
| cttcctgagc gggccgcagg cagcgccctc acccctgtcc cctccccagc ctgcacccac | 481 |
| tccattgaag atgtacccgt ccatgcccgc tttctcccct gggcctggta tcaaggaaga | 541 |
| gtcagtgcca ctgagcatcc tgcagacccc caccccacag cccctgccag gggccctcct | 601 |
| gccacagagc ttcccagccc cagccccacc gcagttcagc tccacccctg tgttaggcta | 661 |
| ccccagccct ccgggaggct tctctacagg aagccctccc gggaacaccc agcagccgct | 721 |
| gcctggcctg ccactggctt ccccgccagg ggtcccgccc gtctccttgc acacccaggt | 781 |
| ccagagtgtg gtcccccagc agctactgac agtcacagct gcccccacgg cagcccctgt | 841 |
| aacgaccact gtgacctcgc agatccagca ggtcccggtc ctgctgcagc cccacttcat | 901 |
| caaggcagac tcgctgcttc tgacagccat gaagacagac ggagccactg tgaaggcggc | 961 |
| aggtctcagt cccctggtct ctggcaccac tgtgcagaca gggcctttgc cgaccctggt | 1021 |
| gagtggcgga accatcttgg caacagtccc actggtcgta gatgcggaga agctgcctat | 1081 |
| caaccggctc gcagctggca gcaaggcccc ggcctctgcc cagagccgtg gagagaagcg | 1141 |
| cacagcccac aacgccattg agaagcgcta ccgctcctcc atcaatgaca aaatcattga | 1201 |
| gctcaaggat ctggtggtgg gcactgaggc aaagctgaat aaatctgctg tcttgcgcaa | 1261 |
| ggccatcgac tacattcgct ttctgcaaca cagcaaccag aaactcaagc aggagaacct | 1321 |
| aagtctgcgc actgctgtcc acaaaagcaa atctctgaag gatctggtgt cggcctgtgg | 1381 |
| cagtggaggg aacacagacg tgctcatgga gggcGtgaag actgaggtgg aggacacact | 1441 |
| gaccccaccc ccctcggatg ctggctcacc tttccagagc agccccttgt cccttggcag | 1501 |
| caggggcagt ggcagcggtg gcagtggcag tgactcggag cctgacagcc cagtctttga | 1561 |
| ggacagcaag gcaaagccag agcagcggcc gtctctgcac agccggggca tgctggaccg | 1621 |
| ctcccgcctg gccctgtgca cgctcgtctt cctctgcctg tcctgcaacc ccttggcctc | 1681 |
| cttgctgggg gcccgggggc ttcccagccc ctcagatacc accagcgtct accatagccc | 1741 |
| tgggcgcaac gtgctgggca ccgagagcag agatggccct ggctgggccc agtggctgct | 1801 |
| gcccccagtg gtctggctgc tcaatgggct gttggtgctc gtctccttgg tgcttctctt | 1861 |
| tgtctacggt gagccagtca cacggcccca ctcaggcccc gccGtgtact tctggaggca | 1921 |
| tcgcaagcag gctgacctgg acctggcccg gggagacttt gcccaggctg cccagcagct | 1981 |
| gtggctggcc ctgcgggcac tgggccggcc cctgcccacc tcccacctgg acctggcttg | 2041 |
| tagcctcctc tggaacctca tccgtcacct gctgcagcgt ctctgggtgg gccgctggct | 2101 |
| ggcaggccgg gcagggggcc tgcagcagga ctgtgctctg cgagtggatg ctagcgccag | 2161 |
| cgcccgagac gcagccctgg tctaccataa gctgcaccag ctgcacacca tggggaagca | 2221 |
| cacaggcggg cacctcactg ccaccaacct ggcgctgagt gccctgaacc tggcagagtg | 2281 |
| tgcaggggat gccgtgtctg tggcgacgct ggccgagatc tatgtggcgg ctgcattgag | 2341 |
| agtgaagacc agtctcccac gggccttgca ttttctgaca cgcttcttcc tgagcagtgc | 2401 |
| ccgccaggcc tgcctggcac agagtggctc agtgcctcct gccatgcagt ggctctgcca | 2461 |
| ccccgtgggc caccgtttct tcgtggatgg ggactggtcc gtgctcagta ccccatggga | 2521 |
| gagcctgtac agcttggccg ggaacccagt ggaccccctg gcccaggtga ctcagctatt | 2581 |
| ccgggaacat ctcttagagc gagcactgaa ctgtgtgacc cagcccaacc ccagccctgg | 2641 |
| gtcagctgat ggggacaagg aattctcgga tgccctcggg tacctgcagc tgctgaacag | 2701 |
| ctgttctgat gctgcggggg ctcctgccta cagcttctcc atcagttcca gcatggccac | 2761 |
| caccaccggc gtagacccgg tggccaagtg gtgggcctct crgacagctg tggtgatcca | 2821 |
| ctggctgcgg cgggatgagg aggcggctga gcggctgtgc ccgctggtgg agcacctgcc | 2881 |
| ccgggtgctg caggagtctg agagacccct gcccagggca gctctgcact ccttcaaggc | 2941 |
| tgcccgggcc ctgctgggct gtgccaaggc agagtctggt ccagccagcc tgaccatctg | 3001 |
| tgagaaggcc agtgggtacc tgcaggacag cctggctacc acaccagcca gcagctccat | 3061 |
| tgacaaggcc gtgcagctgt tcctgtgtga cctgcttctt gtggtgcgca ccagcctgtg | 3121 |
| gcggcagcag cagcccccgg ccccggcccc agcagcccag ggcgccagca gcaggcccca | 3181 |
| ggcttccgcc cttgagctgc gtggcttcca acgggacctg agcagcctga ggcggctggc | 3241 |
| acagagcttc cggcccgcca tgcggagggt gttcctacat gaggccacgg cccggctgat | 3301 |
| ggcgggggcc agccccacac ggacacacca gctcctcgac cgcagtctga ggcggcgggc | 3361 |
| aggccccggt ggcaaaggag gcgcggtggc ggagctggag ccgcggccca cgcggcggga | 3421 |
| gcacgcggag gccttgctgc tggcctcctg ctacctgccc cccggcttcc tgtcggcgcc | 3481 |
| cgggcagcgc gtgggcatgc tggctgaggc ggcgcgcaca ctcgagaagc ttggcgatcg | 3541 |
| ccggctgctg cacgactgtc agcagatgct catgcgcctg ggcggtggga ccactgtcac | 3601 |
| ttccagctag accccgtgtc cccggcctca gcacccctgt ctctagccac tttggtcccg | 3661 |
| tgcagcttct gtcctgcgtc gaagctttga aggccgaagg cagtgcaaga gactctggcc | 3721 |
| tccacagttc gacctgcggc tgctgtgtgc cttcgcggtg gaaggcccga ggggcgcgat | 3781 |
| cttgacccta agaccggcgg ccatgatggt gctgacctct ggtggccgat cggggcactg | 3841 |
| caggggccga gccattttgg ggggcccccc tccttgctct gcaggcacct tagtggcttt | 3901 |
| tttcctcctg tgtacaggga agagaggggt acatttccct gtgctgacgg aagccaactt | 3961 |
| ggctttcccg gactgcaagc agggctctgc cccagaggcc tctctctccg tcgtgggaga | 4021 |
| gagacgtgta catagtgtag gtcagcgtgc ttagcctcct gacctgaggc tcctgtgcta | 4081 |
| ctttgccttt tgcaaacttt attttcatag attgagaagt tttgtacaga gaattaaaaa | 4141 |
| tgaaattatt tata | |

| | |
|---|---|
| SREBP1 Met 417/Val580 allele (Variant) (SEQ ID NO:3) | |
| taacgaggaa cttttcgccg gcgccgggcc gcctctgagg ccagggcagg acacgaacgc | 61 |
| gcggagcggc ggcggcgact gagagccggg gccgcggcgg cgctccctag gaagggccgt | 121 |
| acgaggcggc gggcccggcg ggcctcccgg aggaggcggc tgcgccatgg acgagccacc | 181 |
| cttcagcgag gcggctttgg agcaggcgct gggcgagccg tgcgatctgg acgcggcgct | 241 |
| gctgaccgac atcgaagaca tgcttcagct tatcaacaac caagacagtg acttccctgg | 301 |
| cctatttgac ccaccctatg ctgggagtgg ggcagggggc acagaccctg ccagccccga | 361 |
| taccagctcc ccaggcagct tgtctccacc tcctgccaca ttgagctcct ctcttgaagc | 421 |
| cttcctgagc gggccgcagg cagcgccctc acccctgtcc cctccccagc ctgcacccac | 481 |
| tccattgaag atgtacccgt ccatgcccgc tttctcccct gggcctggta tcaaggaaga | 541 |
| gtcagtgcca ctgagcatcc tgcagacccc caccccacag cccctgccag gggccctcct | 601 |
| gccacagagc ttcccagccc cagccccacc gcagttcagc tccacccctg tgttaggcta | 661 |
| ccccagccct ccgggaggct tctctacagg aagccctccc gggaacaccc agcagccgct | 721 |
| gcctggcctg ccactggctt ccccgccagg ggtcccgccc gtctccttgc acacccaggt | 781 |
| ccagagtgtg gtcccccagc agctactgac agtcacagct gcccccacgg cagcccctgt | 841 |
| aacgaccact gtgacctcgc agatccagca ggtcccggtc ctgctgcagc cccacttcat | 901 |
| caaggcagac tcgctgcttc tgacagccat gaagacagac ggagccactg tgaaggcggc | 961 |
| aggtctcagt cccctggtct ctggcaccac tgtgcagaca gggcctttgc cgaccctggt | 1021 |
| gagtggcgga accatcttgg caacagtccc actggtcgta gatgcggaga agctgcctat | 1081 |
| caaccggctc gcagctggca gcaaggcccc ggcctctgcc cagagccgtg gagagaagcg | 1141 |
| cacagcccac aacgccattg agaagcgcta ccgctcctcc atcaatgaca aaatcattga | 1201 |
| gctcaaggat ctggtggtgg gcactgaggc aaagctgaat aaatctgctg tcttgcgcaa | 1261 |
| ggccatcgac tacattcgct ttctgcaaca cagcaaccag aaactcaagc aggagaacct | 1321 |
| aagtctgcgc actgctgtcc acaaaagcaa atctctgaag gatctggtgt cggcctgtgg | 1381 |
| cagtggaggg aacacagacg tgctcatgga gggcAtgaag actgaggtgg aggacacact | 1441 |
| gaccccaccc ccctcggatg ctggctcacc tttccagagc agccccttgt cccttggcag | 1501 |
| caggggcagt ggcagcggtg gcagtggcag tgactcggag cctgacagcc cagtctttga | 1561 |
| ggacagcaag gcaaagccag agcagcggcc gtctctgcac agccggggca tgctggaccg | 1621 |
| ctcccgcctg gccctgtgca cgctcgtctt cctctgcctg tcctgcaacc ccttggcctc | 1681 |
| cttgctgggg gcccgggggc ttcccagccc ctcagatacc accagcgtct accatagccc | 1741 |
| tgggcgcaac gtgctgggca ccgagagcag agatggccct ggctgggccc agtggctgct | 1801 |
| gcccccagtg gtctggctgc tcaatgggct gttggtgctc gtctccttgg tgcttctctt | 1861 |
| tgtctacggt gagccagtca cacggcccca ctcaggcccc gccGtgtact tctggaggca | 1921 |
| tcgcaagcag gctgacctgg acctggcccg gggagacttt gcccaggctg cccagcagct | 1981 |
| gtggctggcc ctgcgggcac tgggccggcc cctgcccacc tcccacctgg acctggcttg | 2041 |
| tagcctcctc tggaacctca tccgtcacct gctgcagcgt ctctgggtgg gccgctggct | 2101 |
| ggcaggccgg gcagggggcc tgcagcagga ctgtgctctg cgagtggatg ctagcgccag | 2161 |
| cgcccgagac gcagccctgg tctaccataa gctgcaccag ctgcacacca tggggaagca | 2221 |
| cacaggcggg cacctcactg ccaccaacct ggcgctgagt gccctgaacc tggcagagtg | 2281 |
| tgcaggggat gccgtgtctg tggcgacgct ggccgagatc tatgtggcgg ctgcattgag | 2341 |
| agtgaagacc agtctcccac gggccttgca ttttctgaca cgcttcttcc tgagcagtgc | 2401 |
| ccgccaggcc tgcctggcac agagtggctc agtgcctcct gccatgcagt ggctctgcca | 2461 |
| ccccgtgggc caccgtttct tcgtggatgg ggactggtcc gtgctcagta ccccatggga | 2521 |
| gagcctgtac agcttggccg ggaacccagt ggaccccctg gcccaggtga ctcagctatt | 2581 |
| ccgggaacat ctcttagagc gagcactgaa ctgtgtgacc cagcccaacc ccagccctgg | 2641 |
| gtcagctgat ggggacaagg aattctcgga tgccctcggg tacctgcagc tgctgaacag | 2701 |
| ctgttctgat gctgcggggg ctcctgccta cagcttctcc atcagttcca gcatggccac | 2761 |
| caccaccggc gtagacccgg tggccaagtg gtgggcctct ctgacagctg tggtgatcca | 2821 |
| ctggctgcgg cgggatgagg aggcggctga gcggctgtgc ccgctggtgg agcacctgcc | 2881 |
| ccgggtgctg caggagtctg agagacccct gcccagggca gctctgcact ccttcaaggc | 2941 |
| tgcccgggcc ctgctgggct gtgccaaggc agagtctggt ccagccagcc tgaccatctg | 3001 |
| tgagaaggcc agtgggtacc tgcaggacag cctggctacc acaccagcca gcagctccat | 3061 |
| tgacaaggcc gtgcagctgt tcctgtgtga cctgcttctt gtggtgcgca ccagcctgtg | 3121 |
| gcggcagcag cagcccccgg ccccggcccc agcagcccag ggcgccagca gcaggcccca | 3181 |
| ggcttccgcc cttgagctgc gtggcttcca acgggacctg agcagcctga ggcggctggc | 3241 |
| acagagcttc cggcccgcca tgcggagggt gttcctacat gaggccacgg cccggctgat | 3301 |
| ggcgggggcc agccccacac ggacacacca gctcctcgac cgcagtctga ggcggcgggc | 3361 |
| aggccccggt ggcaaaggag gcgcggtggc ggagctggag ccgcggccca cgcggcggga | 3421 |
| gcacgcggag gccttgctgc tggcctcctg ctacctgccc cccggcttcc tgtcggcgcc | 3481 |
| cgggcagcgc gtgggcatgc tggctgaggc ggcgcgcaca ctcgagaagc ttggcgatcg | 3541 |
| ccggctgctg cacgactgtc agcagatgct catgcgcctg ggcggtggga ccactgtcac | 3601 |
| ttccagctag accccgtgtc cccggcctca gcacccctgt ctctagccac tttggtcccg | 3661 |
| tgcagcttct gtcctgcgtc gaagctttga aggccgaagg cagtgcaaga gactctggcc | 3721 |
| tccacagttc gacctgcggc tgctgtgtgc cttcgcggtg gaaggcccga ggggcgcgat | 3781 |
| cttgacccta agaccggcgg ccatgatggt gctgacctct ggtggccgat cggggcactg | 3841 |
| caggggccga gccattttgg ggggcccccc tccttgctct gcaggcacct tagtggcttt | 3901 |
| tttcctcctg tgtacaggga agagaggggt acatttccct gtgctgacgg aagccaactt | 3961 |
| ggctttcccg gactgcaagc agggctctgc cccagaggcc tctctctccg tcgtgggaga | 4021 |
| gagacgtgta catagtgtag gtcagcgtgc ttagcctcct gacctgaggc tcctgtgcta | 4081 |
| ctttgccttt tgcaaacttt attttcatag attgagaagt tttgtacaga gaattaaaaa | 4141 |
| tgaaattatt tata | |

| | |
|---|---|
| SREBP1 Val417/Met580 allele (Variant) (SEQ ID NO:5) | |
| taacgaggaa cttttcgccg gcgccgggcc gcctctgagg ccagggcagg acacgaacgc | 61 |
| gcggagcggc ggcggcgact gagagccggg gccgcggcgg cgctccctag gaagggccgt | 121 |
| acgaggcggc gggcccggcg ggcctcccgg aggaggcggc tgcgccatgg acgagccacc | 181 |
| cttcagcgag gcggctttgg agcaggcgct gggcgagccg tgcgatctgg acgcggcgct | 241 |
| gctgaccgac atcgaagaca tgcttcagct tatcaacaac caagacagtg acttccctgg | 301 |
| cctatttgac ccaccctatg ctgggagtgg ggcagggggc acagaccctg ccagccccga | 361 |
| taccagctcc ccaggcagct tgtctccacc tcctgccaca ttgagctcct ctcttgaagc | 421 |
| cttcctgagc gggccgcagg cagcgccctc acccctgtcc cctccccagc ctgcacccac | 481 |
| tccattgaag atgtacccgt ccatgcccgc tttctcccct gggcctggta tcaaggaaga | 541 |
| gtcagtgcca ctgagcatcc tgcagacccc caccccacag cccctgccag gggccctcct | 601 |
| gccacagagc ttcccagccc cagccccacc gcagttcagc tccacccctg tgttaggcta | 661 |
| ccccagccct ccgggaggct tctctacagg aagccctccc gggaacaccc agcagccgct | 721 |
| gcctggcctg ccactggctt ccccgccagg ggtcccgccc gtctccttgc acacccaggt | 781 |
| ccagagtgtg gtcccccagc agctactgac agtcacagct gcccccacgg cagcccctgt | 841 |
| aacgaccact gtgacctcgc agatccagca ggtcccggtc ctgctgcagc cccacttcat | 901 |
| caaggcagac tcgctgcttc tgacagccat gaagacagac ggagccactg tgaaggcggc | 961 |
| aggtctcagt cccctggtct ctggcaccac tgtgcagaca gggcctttgc cgaccctggt | 1021 |
| gagtggcgga accatcttgg caacagtccc actggtcgta gatgcggaga agctgcctat | 1081 |
| caaccggctc gcagctggca gcaaggcccc ggcctctgcc cagagccgtg gagagaagcg | 1141 |
| cacagcccac aacgccattg agaagcgcta ccgctcctcc atcaatgaca aaatcattga | 1201 |
| gctcaaggat ctggtggtgg gcactgaggc aaagctgaat aaatctgctg tcttgcgcaa | 1261 |
| ggccatcgac tacattcgct ttctgcaaca cagcaaccag aaactcaagc aggagaacct | 1321 |
| aagtctgcgc actgctgtcc acaaaagcaa atctctgaag gatctggtgt cggcctgtgg | 1381 |
| cagtggaggg aacacagacg tgctcatgga gggcGtgaag actgaggtgg aggacacact | 1441 |
| gaccccaccc ccctcggatg ctggctcacc tttccagagc agccccttgt cccttggcag | 1501 |
| caggggcagt ggcagcggtg gcagtggcag tgactcggag cctgacagcc cagtctttga | 1561 |
| ggacagcaag gcaaagccag agcagcggcc gtctctgcac agccggggca tgctggaccg | 1621 |
| ctcccgcctg gccctgtgca cgctcgtctt cctctgcctg tcctgcaacc ccttggcctc | 1681 |
| cttgctgggg gcccgggggc ttcccagccc ctcagatacc accagcgtct accatagccc | 1741 |
| tgggcgcaac gtgctgggca ccgagagcag agatggccct ggctgggccc agtggctgct | 1801 |
| gcccccagtg gtctggctgc tcaatgggct gttggtgctc gtctccttgg tgcttctctt | 1861 |
| tgtctacggt gagccagtca cacggcccca ctcaggcccc gccAtgtact tctggaggca | 1921 |
| tcgcaagcag gctgacctgg acctggcccg gggagacttt gcccaggctg cccagcagct | 1981 |
| gtggctggcc ctgcgggcac tgggccggcc cctgcccacc tcccacctgg acctggcttg | 2041 |
| tagcctcctc tggaacctca tccgtcacct gctgcagcgt ctctgggtgg gccgctggct | 2101 |
| ggcaggccgg gcagggggcc tgcagcagga ctgtgctctg cgagtggatg ctagcgccag | 2161 |
| cgcccgagac gcagccctgg tctaccataa gctgcaccag ctgcacacca tggggaagca | 2221 |
| cacaggcggg cacctcactg ccaccaacct ggcgctgagt gccctgaacc tggcagagtg | 2281 |
| tgcaggggat gccgtgtctg tggcgacgct ggccgagatc tatgtggcgg ctgcattgag | 2341 |
| agtgaagacc agtctcccac gggccttgca ttttctgaca cgcttcttcc tgagcagtgc | 2401 |
| ccgccaggcc tgcctggcac agagtggctc agtgcctcct gccatgcagt ggctctgcca | 2461 |
| ccccgtgggc caccgtttct tcgtggatgg ggactggtcc gtgctcagta ccccatggga | 2521 |
| gagcctgtac agcttggccg ggaacccagt ggaccccctg gcccaggtga ctcagctatt | 2581 |
| ccgggaacat ctcttagagc gagcactgaa ctgtgtgacc cagcccaacc ccagccctgg | 2641 |
| gtcagctgat ggggacaagg aattctcgga tgccctcggg tacctgcagc tgctgaacag | 2701 |
| ctgttctgat gctgcggggg ctcctgccta cagcttctcc atcagttcca gcatggccac | 2761 |
| caccaccggc gtagacccgg tggccaagtg gtgggcctct ctgacagctg tggtgatcca | 2821 |
| ctggctgcgg cgggatgagg aggcggctga gcggctgtgc ccgctggtgg agcacctgcc | 2881 |
| ccgggtgctg caggagtctg agagacccct gcccagggca gctctgcact ccttcaaggc | 2941 |
| tgcccgggcc ctgctgggct gtgccaaggc agagtctggt ccagccagcc tgaccatctg | 3001 |
| tgagaaggcc agtgggtacc tgcaggacag cctggctacc acaccagcca gcagctccat | 3061 |
| tgacaaggcc gtgcagctgt tcctgtgtga cctgcttctt gtggtgcgca ccagcctgtg | 3121 |
| gcggcagcag cagcccccgg ccccggcccc agcagcccag ggcgccagca gcaggcccca | 3181 |
| ggcttccgcc cttgagctgc gtggcttcca acgggacctg agcagcctga ggcggctggc | 3241 |
| acagagcttc cggcccgcca tgcggagggt gttcctacat gaggccacgg cccggctgat | 3301 |
| ggcgggggcc agccccacac ggacacacca gctcctcgac cgcagtctga ggcggcgggc | 3361 |
| aggccccggt ggcaaaggag gcgcggtggc ggagctggag ccgcggccca cgcggcggga | 3421 |
| gcacgcggag gccttgctgc tggcctcctg ctacctgccc cccggcttcc tgtcggcgcc | 3481 |
| cgggcagcgc gtgggcatgc tggctgaggc ggcgcgcaca ctcgagaagc ttggcgatcg | 3541 |
| ccggctgctg cacgactgtc agcagatgct catgcgcctg ggcggtggga ccactgtcac | 3601 |
| ttccagctag accccgtgtc cccggcctca gcacccctgt ctctagccac tttggtcccg | 3661 |
| tgcagcttct gtcctgcgtc gaagctttga aggccgaagg cagtgcaaga gactctggcc | 3721 |
| tccacagttc gacctgcggc tgctgtgtgc cttcgcggtg gaaggcccga ggggcgcgat | 3781 |
| cttgacccta agaccggcgg ccatgatggt gctgacctct ggtggccgat cggggcactg | 3841 |
| caggggccga gccattttgg ggggcccccc tccttgctct gcaggcacct tagtggcttt | 3901 |
| tttcctcctg tgtacaggga agagaggggt acatttccct gtgctgacgg aagccaactt | 3961 |
| ggctttcccg gactgcaagc agggctctgc cccagaggcc tctctctccg tcgtgggaga | 4021 |
| gagacgtgta catagtgtag gtcagcgtgc ttagcctcct gacctgaggc tcctgtgcta | 4081 |
| ctttgccttt tgcaaacttt attttcatag attgagaagt rttgtacaga gaattaaaaa | 4141 |
| tgaaattatt tata | |

| | |
|---|---|
| SREBP1 Met417/Met580 allele (Predictcd Variant) (SEQ ID NO:7) | |
| taacgaggaa cttttcgccg gcgccgggcc gcctctgagg ccagggcagg acacgaacgc | 61 |
| gcggagcggc ggcggcgact gagagccggg gccgcggcgg cgctccctag gaagggccgt | 121 |
| acgaggcggc gggcccggcg ggcctcccgg aggaggcggc tgcgccatgg acgagccacc | 181 |
| cttcagcgag gcggctttgg agcaggcgct gggcgagccg tgcgatctgg acgcggcgct | 241 |
| gctgaccgac atcgaagaca tgcttcagct tatcaacaac caagacagtg acttccctgg | 301 |
| cctatttgac ccaccctatg ctgggagtgg ggcagggggc acagaccctg ccagccccga | 361 |
| taccagctcc ccaggcagct tgtctccacc tcctgccaca ttgagctcct ctcttgaagc | 421 |
| cttcctgagc gggccgcagg cagcgccctc acccctgtcc cctccccagc ctgcacccac | 481 |
| tccattgaag atgtacccgt ccatgcccgc tttctcccct gggcctggta tcaaggaaga | 541 |
| gtcagtgcca ctgagcatcc tgcagacccc caccccacag cccctgccag gggccctcct | 601 |
| gccacagagc ttcccagccc cagccccacc gcagttcagc tccacccctg tgttaggcta | 661 |
| ccccagccct ccgggaggct tctctacagg aagccctccc gggaacaccc agcagccgct | 721 |
| gcctggcctg ccactggctt ccccgccagg ggtcccgccc gtctccttgc acacccaggt | 781 |
| ccagagtgtg gtcccccagc agctactgac agtcacagct gcccccacgg cagcccctgt | 841 |
| aacgaccact gtgacctcgc agatccagca ggtcccggtc ctgctgcagc cccacttcat | 901 |
| caaggcagac tcgctgcttc tgacagccat gaagacagac ggagccactg tgaaggcggc | 961 |
| aggtctcagt cccctggtct ctggcaccac tgtgcagaca gggcctttgc cgaccctggt | 1021 |
| gagtggcgga accatcttgg caacagtccc actggtcgta gatgcggaga agctgcctat | 1081 |
| caaccggctc gcagctggca gcaaggcccc ggcctctgcc cagagccgtg gagagaagcg | 1141 |
| cacagcccac aacgccattg agaagcgcta ccgctcctcc atcaatgaca aaatcattga | 1201 |
| gctcaaggat ctggtggtgg gcactgaggc aaagctgaat aaatctgctg tcttgcgcaa | 1261 |
| ggccatcgac tacattcgct ttctgcaaca cagcaaccag aaactcaagc aggagaacct | 1321 |
| aagtctgcgc actgctgtcc acaaaagcaa atctctgaag gatctggtgt cggcctgtgg | 1381 |
| cagtggaggg aacacagacg tgctcatgga gggcAtgaag actgaggtgg aggacacact | 1441 |
| gaccccaccc ccctcggatg ctggctcacc tttccagagc agccccttgt cccttggcag | 1501 |
| caggggcagt ggcagcggtg gcagtggcag tgactcggag cctgacagcc cagtctttga | 1561 |
| ggacagcaag gcaaagccag agcagcggcc grctctgcac agccggggca tgctggaccg | 1621 |
| ctcccgcctg gccctgtgca cgctcgtctt cctctgcctg tcctgcaacc ccttggcctc | 1681 |
| cttgctgggg gcccgggggc ttcccagccc ctcagatacc accagcgtct accatagccc | 1741 |
| tgggcgcaac gtgctgggca ccgagagcag agatggccct ggctgggccc agtggctgct | 1801 |
| gcccccagtg gtctggctgc tcaatgggct gttggtgctc gtctccttgg tgcttctctt | 1861 |
| tgtctacggt gagccagtca cacggcccca ctcaggcccc gccAtgtact tctggaggca | 1921 |
| tcgcaagcag gctgacctgg acctggcccg gggagacttt gcccaggctg cccagcagct | 1981 |
| gtggctggcc ctgcgggcac tgggccggcc cctgcccacc tcccacctgg acctggcttg | 2041 |
| tagcctcctc tggaacctca tccgtcacct gctgcagcgt ctctgggtgg gccgctggct | 2101 |
| ggcaggccgg gcagggggcc tgcagcagga ctgtgctctg cgagtggatg ctagcgccag | 2161 |
| cgcccgagac gcagccctgg tctaccataa gctgcaccag ctgcacacca tggggaagca | 2221 |
| cacaggcggg cacctcactg ccaccaacct ggcgctgagt gccctgaacc tggcagagtg | 2281 |
| tgcaggggat gccgtgtctg tggcgacgct ggccgagatc tatgtggcgg ctgcattgag | 2341 |
| agtgaagacc agtctcccac gggccttgca ttttctgaca cgcttcttcc tgagcagtgc | 2401 |
| ccgccaggcc tgcctggcac agagtggctc agtgcctcct gccatgcagt ggctctgcca | 2461 |
| ccccgtgggc caccgtttct tcgtggatgg ggactggtcc gtgctcagta ccccatggga | 2521 |
| gagcctgtac agcttggccg ggaacccagt ggaccccctg gcccaggtga ctcagctatt | 2581 |
| ccgggaacat ctcttagagc gagcactgaa ctgtgtgacc cagcccaacc ccagccctgg | 2641 |
| gtcagctgat ggggacaagg aattctcgga tgccctcggg tacctgcagc tgctgaacag | 2701 |
| ctgttctgat gctgcggggg ctcctgccta cagcttctcc atcagttcca gcatggccac | 2761 |
| caccaccggc gtagacccgg tggccaagtg gtgggcctct ctgacagctg tggtgatcca | 2821 |
| ctggctgcgg cgggatgagg aggcggctga gcggctgtgc ccgctggtgg agcacctgcc | 2881 |
| ccgggtgctg caggagtctg agagacccct gcccagggca gctctgcact ccttcaaggc | 2941 |
| tgcccgggcc ctgctgggct gtgccaaggc agagtctggt ccagccagcc tgaccatctg | 3001 |
| tgagaaggcc agtgggtacc tgcaggacag cctggctacc acaccagcca gcagctccat | 3061 |
| tgacaaggcc gtgcagctgt tcctgtgtga cctgcttctt gtggtgcgca ccagcctgtg | 3121 |
| gcggcagcag cagcccccgg ccccggcccc agcagcccag ggcgccagca gcaggcccca | 3181 |
| ggcttccgcc cttgagctgc gtggcttcca acgggacctg agcagcctga ggcggctggc | 3241 |
| acagagcttc cggcccgcca tgcggagggt gttcctacat gaggccacgg cccggctgat | 3301 |
| ggcgggggcc agccccacac ggacacacca gctcctcgac cgcagtctga ggcggcgggc | 3361 |
| aggccccggt ggcaaaggag gcgcggtggc ggagctggag ccgcggccca cgcggcggga | 3421 |
| gcacgcggag gccttgctgc tggcctcctg ctacctgccc cccggcttcc tgtcggcgcc | 3481 |
| cgggcagcgc gtgggcatgc tggctgaggc ggcgcgcaca ctcgagaagc ttggcgatcg | 3541 |
| ccggctgctg cacgactgtc agcagatgct catgcgcctg ggcggtggga ccactgtcac | 3601 |
| ttccagctag accccgtgtc cccggcctca gcacccctgt ctctagccac tttggtcccg | 3661 |
| tgcagcttct gtcctgcgtc gaagctttga aggccgaagg cagtgcaaga gactctggcc | 3721 |
| tccacagttc gacctgcggc tgctgtgtgc cttcgcggtg gaaggcccga ggggcgcgat | 3781 |
| cttgacccta agaccggcgg ccatgatggt gctgacctct ggtggccgat cggggcactg | 3841 |
| caggggccga gccattttgg ggggcccccc tccttgctct gcaggcacct tagtggcttt | 3901 |
| tttcctcctg tgtacaggga agagaggggt acatttccct gtgctgacgg aagccaactt | 3961 |
| ggctttcccg gactgcaagc agggctctgc cccagaggcc tctctctccg tcgtgggaga | 4021 |
| gagacgtgta catagtgtag gtcagcgtgc ttagcctcct gacctgaggc tcctgtgcta | 4081 |
| ctttgccttt tgcaaacttt attttcatag attgagaagt tttgtacaga gaattaaaaa | 4141 |
| tgaaattatt tata | |

These alleles (i.e., SEQ ID NOs:3, 5 and 7) can be genotyped using standard techniques known to those of ordinary skill in the art. The sequences of the probes and primers that can be employed in a genotyping assay are:

For the V417M SNP, probes and primers are as follows.

| | | |
|---|---|---|
| Probes: | CCTCAGTCTTCACGCCCTCCATGA | (SEQ ID NO:9) |
| | CCACCTCAGTCTTCATGCCCTCCAT | (SEQ ID NO:10) |
| Primers: | CTCTGTTCCTTTCGGCCCA | (SEQ ID NO:11) |
| | AAAGGTGAGCCAGCATCCG | (SEQ ID NO:12) |

For the V580M SNP, probes and primers are as follows.

| | | |
|---|---|---|
| Probes: | ACTCAGGCCCCGCCGTGTACTTCT | (SEQ ID NO:13) |
| | CACTCAGGCCCCGCCATGTACTTCT | (SEQ ID NO:14) |
| Primers: | TTGTCTACGGTGAGCCAGTCA | (SEQ ID NO:15) |
| | AGGTCAGCCTGCTTGCGA | (SEQ ID NO:16) |

Generally DNA samples can be genotyped using known techniques (see, e.g., Ranade et al., (2001) Genome Res. 11:1262-1268). The protocol employed in the present Laboratory Example is generally as follows.

A reaction mixture is prepared comprising 30 ng genomic DNA, 900 nM of each primer, 100 nM of each probe in a 5 μL PCR reaction.

PCR amplification is then performed. Representative PCR cycling conditions are: 50° C., 2 minutes then at 95° C., 10 minutes, followed by 40 15 seconds cycles at 94° C., and 1 minute at 62° C. A Perkin-Elmer 9600 thermal cycler can be employed in the PCR reactions (Perkin-Elmer, Wellesley, Mass., USA).

Fluorescence in each well is measured before and after PCR using an ABI Prism® 7700 or ABI Prism® 7900HT Sequence Detection (Applied Biosystems, Foster City, Calif., USA) instrument.

### Example 2

### SNP Functional Assay: SREBP1 Val417Met SNP

SREBP1 bearing the Val417 (reference), Met417 (polymorphism) and/or Met580 (polymorphism) codons can be prepared as described herein. The consequence of the Met417 and/or the Met580 substitution on SREBP1 function can be examined using methods described by Sato et al. (Sato et al., (1994) J. Biol. Chem. 269:17267-17273).

Briefly, these SREBP1 alleles can be cloned into an expression vector (e.g., pCMV, available from Invitrogen, Carlsbad, Calif., USA). Human embryonic kidney cells (HEK293) can be transfected with 0-1 μg of these SREBP1 expressing vectors and a vector carrying a CAT-reporter construct containing the CAT gene under control of the promoter for the human LDL receptor gene. After 4 hours the cells can be re-fed with Dulbecco's modified Eagle's medium containing 10% calf lipoprotein-deficient serum. After 48 h cells can be processed for CAT activity and assayed by the xylene extraction method.

### Example 3

### Engineering the Allelic Forms of a SREBP1 Candidate Gene of the Present Invention

Aside from isolating the allelic genes of the present invention from DNA samples obtained from a human population, as described herein, the invention also encompasses methods of engineering the allelic genes of the present invention through the application of site-directed mutagenesis to the isolated native forms of the genes. Such methodology can applied to synthesize allelic forms of the genes comprising at least one, or more, of the encoding SNPs of the present invention (e.g., silent, missense)—for example at least 1, 2, 3, or 4 encoding SNPs for each gene.

As described herein, isolating a novel SREBP1 allele of the present invention is within the ordinary skill of an artisan trained in the molecular biology arts. Nonetheless, a detailed exemplary method of engineering at least one of the SREBP1 alleles to comprise an encoding and/or non-coding polymorphic nucleic acid sequence, in this case a variant form (e.g., Val417Met and/or Val580Met) of SREBP1 protein (described in Sato et al., (1994) J. Biol. Chem. 269:17267-17273) is provided. In one example, cDNA clones encoding the human SREBP1 protein can be identified by homology searches with the BLASTN program (Altschul et al., (1990) J. Mol. Biol. 215: 403-10) against a Genbank non-redundant nucleotide sequence database using the published human SREBP1 cDNA. After obtaining these clones, they can be sequenced to confirm the validity of the DNA sequences.

Once these clones are confirmed to contain the intact wild type cDNA sequence of the SREBP1 coding region, a Val417Met and/or a Val580Met polymorphism (mutation) can be introduced into the native sequence using PCR directed in vitro mutagenesis (see, e.g., Cormack & Castano, (2002) Method. Enzymol. 350:199-218). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequence analysis. This method generally comprises the following steps.

1. Subcloning the cDNA insert into a high copy plasmid vector containing multiple cloning sites and M13 flanking sequences, such as pUC19 (Sambrook et al., Molecular Cloning: A Laboratory Manual, (3^{rd} ed.) Cold Spring Harbor Laboratory Press, Cold Spring Harbor, N.Y., USA (2001)), in the forward orientation. Other plasmids can also be employed, and can be desirable in certain circumstances.

2. Introducing a mutation by PCR amplification of the cDNA region downstream of the mutation site using a primer comprising the mutation. (see, e.g., FIG. 8.5.2 in Cormack & Castano, (2002) Method. Enzymol. 350:199-218). When introducing a Val417Met mutation into the human SREBP1 protein, the following two primers can be employed:

| | |
|---|---|
| M13 reversc sequencing primer: | |
| 5′-AGCGGATAACAATTTCACACAGGA-3′. | (SEQ ID NO:17) |
| Mutation primer: | |
| 5′-ACTCAGGCCCCGCCGTGTACTTCT-3′ | (SEQ ID NO:13) |
| or | (Val580Met) |
| 5′-CCTCAGTCTTCACGCCCTCCATGA-3′ | (SEQ ID NO:9) |
| | (Val417Met) |

The mutation primer can comprises a mutation (Val417Met or Val580Met). The M13 reverse sequencing primer hybridizes to the pUC19 vector. Subcloned cDNA comprising the human SREBP1 protein is used as a template (described in Step 1 of the present Laboratory Example).

A 100 μl PCR reaction mixture is prepared using 10 ng of the template DNA, 200 μM 4dNTPs, 1 μM primers, 0.25 U Taq DNA polymerase (PE), and a standard Taq DNA polymerase buffer. Typical PCR cycling condition are as follows:
20-25 cycles: 45 sec, 93 degrees
2 min, 50 degrees
2 min, 72 degrees
1 cycle: 10 min, 72 degrees

After the final extension step of PCR, 5 U Klenow Fragment is added and incubated for 15 min at 30° C. The PCR product is then digested with the restriction enzyme, EcoRI.

3. Performing PCR amplification of the upstream region, using subcloned cDNA as a template (the product of Step 1). This PCR is done using the following two primers:

| | |
|---|---|
| M13 forward sequencing primer: | |
| 5′-CGCCAGGGTTTTCCCAGTCACGAC-3′. | (SEQ ID NO:18) |
| Flanking primer: | |
| 5′-GCCCTCCATGAGCACGTCTGTGTTCCC-3′. | (SEQ ID NO:19) |

The flanking primer is complementary to the upstream flanking sequence of the Val417Met mutation. M13 forward sequencing primer hybridizes to the pUC19 vector. PCR conditions and Klenow treatments can be those provided in Step 2, above. The PCR product is then digested with the restriction enzyme, HindIII.

4. Preparing the pUC19 vector for cloning the cDNA comprising the polymorphic site. The pUC19 plasmid DNA is digested with EcoRI and HindIII. The resulting digested vector fragment can then be purified using techniques well known in the art, such as gel purification, for example.

5. Combining and ligating the products from Step 2 (PCR product containing mutation), Step 3 (PCR product containing the upstream region), and Step 4 (digested vector), using standard blunt-end ligation conditions (Sambrook et al., Molecular Cloning: A Laboratory Manual, (3^{rd} ed.) Cold Spring Harbor Laboratory Press, Cold Spring Harbor, N.Y., USA (2001)).

6. Transforming E. coli cells with the resulting recombinant plasmid from Step 5 using methods known in the art, such as, for example, the transformation methods described in Sambrook et al., Molecular Cloning: A Laboratory Manual, (3^{rd} ed.) Cold Spring Harbor Laboratory Press, Cold Spring Harbor, N.Y., USA (2001).

7. Analyzing the amplified fragment portion of the plasmid DNA by DNA sequencing to confirm the presence of the point mutation, and the absence of any other mutations introduced during PCR. Techniques and methods of sequencing the insert DNA, including the primers utilized, are described herein or are otherwise known in the art.

Those of ordinary skill in the art will appreciate that the methods of the present Example can be applied to engineering any polymorphic gene of the present invention through the substitution of applicable mutation, flanking, PCR, and sequencing primers for each specific gene and/or polymorphism. Some of these primers can be selected from any one of the applicable primers provided herein, can be designed using the Primer3 program (Rozen & Skaletzky, in Bioinformatics Methods and Protocols: Methods in Molecular Biology (Krawetz & Misener, eds.), Humana Press, Totowa, N.J., USA, (2000) pp 365-386), or designed manually, as described. Such primers can comprise at least a portion of any one of the polynucleotide sequences of the present invention.

Moreover, those of ordinary skill in the art will appreciate that the above method can be applied to engineering more than one polymorphic nucleic acid sequence of the present invention. Such an engineered gene can be created through successive rounds of site-directed mutagenesis, as described in Steps 1 through 7 above, or consolidated into a single round of mutagenesis. For example, Step 2 above can be performed for each mutation, then the products of both mutation amplifications can be combined with the product of Step 3 and 4, and the procedure followed as described.

### Example 4

### Method of Genotyping a SNP of the Present Invention

(a) Genomic DNA Preparation

Genomic DNA samples for genotyping can be prepared using the PURIGENE™ DNA extraction kit from Gentra Systems (Minneapolis, Minn., USA). After preparation, DNA samples can be diluted to a 2 ng/μl working concentration with TE buffer (10 mM Tris-Cl, pH 8.0, 0.1 mM EDTA, pH 8.0) and stored in 1 ml 96 deep well plates (VWR Scientific Products, West Chester, Pa., USA) at −20° C. until use.

Samples for genomic DNA preparations can be obtained from patients participating in a clinical study, or from other sources known in the art or otherwise described herein.

(b) Genotyping

The SNP genotyping reactions may be performed using the SNPSTREAM™ system (Orchid Bioscience, Princeton, N.J., USA) based on genetic bit analysis (Nikiforov et al., (1994) Nucl. Acid Res. 22:4167-75).

The regions including polymorphic sites can be amplified by the polymerase chain reaction (PCR) using a pair of primers (OPERON Technologies, Alameda, Calif.), one of which is phosphorothioated. 6 ml PCR cocktail containing 1.0 ng/μl genomic DNA, 200 μM dNTPs, 0.5 mM forward PCR primer, 0.5 μM reverse PCR primer (phosphorothioated), 0.05 u/μl Platinum Taq DNA polymerase (LifeTechnologies, Rockville, Md.), and 1.5 mM MgCl₂. PCR primer pairs that can be used for genotyping analysis are provided herein. The PCR reaction can be set up in 384-well plates (MJ Research, Waltham, Mass.) using a MINITRAK liquid handling station (Packard Bioscience, Meriden, Conn.). The PCR primer sequences can be selected from those provided herein, or any other primer as may otherwise be required. PCR thermocycling may be performed under the following conditions in a MJ Research (Waltham, Mass.) TETRAD machine: step 1, 95 degrees for 2 min; step 2, 94 degrees for 30 min; step 3, 55 degrees for 2 min; step 4, 72 degrees for 30 sec; step 5, go back to step 2 for an additional 39 cycles; step 6, 72 degrees for 1 min; and step 7, 12 degrees indefinitely

After thermocycling, the amplified samples can be placed in the SNPSTREAM™ (Orchid Bioscience, Princeton, N.J., USA) machine, and automated genetic bit analysis (GBA) (Nikiforov et al., (1994) Nucl. Acid Res. 22:4167-75) reaction can then be performed. The first step of this reaction is degradation of one of the strands of the PCR products by T7 gene 6 exonuclease to make them single-stranded. The strand containing the phosphorothioated primer are resistant to T7 gene 6 nuclease, and were not degraded by this enzyme. After digestion, the single-stranded PCR products are subjected to an annealing step whereby the single stranded PCR products are annealed to the GBA primer on a solid phase, and then subjected to the GBA reaction (single base extension) using dideoxy-NTPs labeled with biotin or fluorescein. GBA primers useful for single base extension are provided in herein. C3 linkers (C3 spacer phosphoramidite) can be incorporated during synthesis of the primer. Such linkers can be obtained from Research Genetics, and Sigma-Genosys, for example. Incorporation of these dideoxynucleotides into a GBA primer are detected by a two color ELISA assay using anti-fluorescein alkaline phosphatase conjugate and anti-biotin horseradish peroxidase. Automated genotype calls are made by GENOPAK™ software (Orchid Bioscience, Princeton, N.J., USA), before manual correction of automated calls are done upon inspection of the resulting allelogram of each SNP.

### Example 5

### Alternative Method of Genotyping a SNP of the Present Invention

In addition to the methods of genotyping described herein, the skilled artisan could determine the genotype of the polymorphisms of the present invention using the herein described alternative method. This method is referred to as the “GBS method” herein and can be performed as described in conjunction with the teachings described elsewhere herein.

Briefly, the direct analysis of the sequence of the polymorphisms of the present invention can be accomplished by DNA. sequencing of PCR products corresponding to the same. PCR amplicons are designed to be in close proximity to the polymorphisms of the present invention using the Primer3 program (Rozen & Skaletzky, in Bioinformatics Methods and Protocols: Methods in Molecular Biology (Krawetz & Misener, eds.), Humana Press, Totowa, N.J., USA, (2000) pp 365-386). An M13 Sequence (e.g., CGCCAGGGTTTTCCCAGTCACGAC (SEQ ID NO:18)) is prepended to each forward PCR primer and an M13 sequence (e.g., AGCGGATAACAATTTCACACAGGA (SEQ ID NO:17)) is prepended to each reverse PCR primer. The specific forward and reverse PCR primers for each SNP of the present invention are provided herein.

PCR amplification can be performed on genomic DNA samples amplified from (20 ng) in reactions (50 μl) containing 10 mM Tris-Cl pH 8.3, 50 mM KCl, 2.5 mM MgCl₂, 150 μM dNTPs, 3 μM PCR primers, and 3.75 U TaqGold DNA polymerase (PE Biosystems, Foster City, Calif.). PCR can then be performed in MJ Research (Waltham, Mass.) TETRAD machines under a cycling condition of 94 degrees 10 min, 30 cycles of 94 degrees 30 sec, 60 degrees 30 sec, and 72 degrees 30 sec, followed by 72 degrees 7 min. PCR products can then be purified using QIAQUICK PCR purification kit (Qiagen), and sequenced by the dye-terminator method using PRISM 3700 automated DNA sequencer (Applied Biosystems, Foster City, Calif.) following the manufacturer's instruction outlined in the Owner's Manual (which is hereby incorporated herein by reference in its entirety) or as described in herein.

PCR products are sequenced by the dye-terminator method using the M13 primers above. The genotype can be determined by analysis of the sequencing results at the polymorphic position.

### Example 6

### Method of Isolating Polymorphic Forms of Candidate Genes of the Present Invention

Since the allelic genes of the present invention represent genes present within at least a subset of the human population, these genes can be isolated using the methods provided herein. For example, the source DNA used to isolate the allelic gene can be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source can be used to isolate the allelic form of the gene(s). The invention encompasses the isolation of such allelic genes from both genomic and/or cDNA libraries created from such source(s).

Next, lymphoblastoid cell lines from these individuals may be obtained from the Coriell Institute. These cells can be grown in RPMI-1640 medium with L-glutamine plus 10% FCS at 37 degrees. PolyA+ RNA are then isolated from these cells using Oligotex Direct Kit (Life Technologies, Rockville, Md.).

First strand cDNA (complementary DNA) is produced using Superscript Preamplification System for First Strand cDNA Synthesis (Life Technologies, Cat No 18089-011) using these polyA+ RNA as templates, as specified in the users manual which is hereby incorporated herein by reference in its entirety. Specific cDNA encoding the protein of interest is amplified by polymerase chain reaction (PCR) using a forward primer which hybridizes to the 5′-UTR region, a reverse primer which hybridizes to the 3′-UTR region, and these first strand cDNA as templates (Sambrook et al., Molecular Cloning: A Laboratory Manual, (3^{rd} ed.) Cold Spring Harbor Laboratory Press, Cold Spring Harbor, N.Y., USA (2001)). For example, the primers specified herein can be used. Alternatively, these primers may be designed using Primer3 program (Rozen & Skaletzky, in Bioinformatics Methods and Protocols: Methods in Molecular Biology (Krawetz & Misener, eds.), Humana Press, Totowa, N.J., USA, (2000) pp 365-386). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook et al., Molecular Cloning: A Laboratory Manual, (3^{rd} ed.) Cold Spring Harbor Laboratory Press, Cold Spring Harbor, N.Y., USA (2001)), plasmid DNA is isolated from these bacterial cells. This plasmid DNA is sequenced to confirm the presence an intact (full-length) coding region of the human SREBP1 protein with desired variation (e.g., V417M and/or V580M) using methods known in the art and described elsewhere herein.

The skilled artisan will appreciate that the above method can be applied to the isolation of other novel polymorphic SREB1 genes of the present invention through the simple substitution of applicable PCR and sequencing primers. Such primers can be selected from any one of the applicable primers provided herein, or may be designed using the Primer3 program (program (Rozen & Skaletzky, in Bioinformatics Methods and Protocols: Methods in Molecular Biology (Krawetz & Misener, eds.), Humana Press, Totowa, N.J., USA, (2000)) as described. Such primers can comprise at least a portion of any one of the polynucleotide sequences of the present invention.

### Example 7

### Alternative Methods of Detecting Polymorphisms Encompassed by the Present Invention

(a) Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below employ amplification of DNA from target samples. This can be accomplished by employing any or a range of methods, for example PCR. See generally PCR Technology: Principles and Applications for DNA Amplification, (Erlich, ed.) Freeman Press, New York, N.Y., (1992); PCR Protocols: a Guide to Methods and Applications, (Innis, et al., eds.), Academic Press, San Diego, Calif., (1990); Mattila et al., (1991) Nucl. Acid Res. 19:4967; Eckert et al., in PCR Methods and Applications, Cold Spring Harbor Laboratory Press, Cold Spring Harbor, N.Y. (1991); PCR (McPherson et al., eds) IRL Press, Oxford, UK; and U.S. Pat. No. 4,683,202.

Other suitable amplification methods include the ligase chain reaction (LCR) (see Wu & Wallace, (1989) Genomics 4:560, Landegren et al., (1988) Science 241:1077, transcription amplification (Kwoh et al., (1989) Proc. Natl. Acad. Sci. USA 86:1173, and self-sustained sequence replication (Guatelli et al., (1990) Proc. Nat. Acad. Sci. USA 87:1874) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Additional methods of amplification are known in the art or are described elsewhere herein.

(b) Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. Additional methods of analysis are known in the art and/or are described elsewhere herein.

1. Allele-Specific Probes

The design and use of allele-specific probes for analyzing polymorphisms is described by e.g., Saiki et al., (1986) Nature 324,163-166; EP 235,726; PCT Publication WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

Polymorphisms can also be identified by hybridization to nucleic acid arrays, some examples of which are described in PCT Publication WO 95/11995. The same arrays or different arrays can be used for analysis of characterized polymorphisms. PCT Publication WO 95/11995 also describes sub-arrays that are optimized for detection of a variant form of a precharacterized polymorphism. Such a sub-array contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to bases).

3. Allele-Specific Primers

An allele-specific primer hybridizes to a site on target DNA overlapping a polymorphism and only primes amplification of an allelic form to which the primer exhibits perfect complementarity. See Gibbs, (1989) Nucleic Acid Res. 17:2427-2448. This primer is used in conjunction with a second primer that hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., PCT Publication WO 93/22456).

4. Direct-Sequencing

The direct analysis of the sequence of polymorphisms of the present invention can be accomplished using either the dideoxy chain termination method or the Maxim-Gilbert method (see Sambrook et al., Molecular Cloning: A Laboratory Manual, (3^{rd} ed.) Cold Spring Harbor Laboratory Press, Cold Spring Harbor, N.Y., USA (2001); Zyskind et al., Recombinant DNA Laboratory Manual, Academic Press, New York (1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g., PCR Technology. Principles and Applications for DNA Amplification, (Erlich, ed.) W.H. Freeman, New York, N.Y. (1992), Chapter 7).

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., (1989) Proc. Nat. Acad. Sci. USA 86:2766-2770. Amplified PCR products can be generated as described above, and heated or otherwise denatured, to form single stranded amplification products. Single-stranded nucleic acids may refold or form secondary structures that are partially dependent on the base sequence. The different electrophoretic mobilities of single-stranded amplification products can be related to base-sequence differences between alleles of target sequences.

7. Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method employed, such as that described by Chen et al., (Chen et al., (1997) Proc. Natl. Acad. Sci. USA 94:10756-61), uses a locus-specific oligonucleotide primer labeled on the 5′ terminus with 5-carboxyfluorescein. This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 8

### Bacterial Expression of a Polypeptide

A polynucleotide encoding a polypeptide of the present invention can be amplified using PCR oligonucleotide primers corresponding to the 5′ and 3′ ends of the DNA sequence, as outlined in the Examples herein or otherwise known in the art, to synthesize insertion fragments. The primers used to amplify the cDNA insert preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen Inc., Chatsworth, Calif., USA). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 μg/ml) and Kan (25 μg/ml). The O/N culture is used to inoculate a large culture at a ratio of 1:100 to 1:250. The cells are grown to an optical density 600 (OD₆₀₀) of between 0.4 and 0.6. IPTG (isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6M guanidine HCl by stirring for 3-4 hours at 4 degree C. The cell debris is removed by centrifugation, and the supernatant containing the polypeptide is loaded onto a nickel-nitrilo-tri-acetic acid (Ni-NTA) affinity resin column (available from QIAGEN, Inc., Chatsworth, Calif., USA). Proteins with a 6×His tag bind to the Ni-NTA resin with high affinity and can be purified in a simple one-step procedure (for details see: “The QIAexpressionist” (1995) QIAGEN, Inc., Chatsworth, Calif., USA).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. T he renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 9

### Purification of a Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (e.g., such as those available from Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., filters available from Filtron, Northboro, Mass.), equilibrated with 40 mM sodium acetate, pH 6.0 is employed. The filtered sample is loaded onto a cation exchange resin (e.g., Poros HS-50, Perceptive Biosystems, Foster City, Calif.). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems, Foster City, Calif.) and weak anion (Poros CM-20, Perceptive Biosystems, Foster City, Calif.) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 μg of purified protein is loaded. The purified protein can also be tested for endotoxin/LPS contamination, and typically the LPS content is less than 0.1 ng/ml according to LAL assays.

### Example 10

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which can include, for example BamHI, Xba I and Asp718. The polyadenylation site of the simian virus 40 (SV40) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one of ordinary skill in the art will readily appreciate, as long as the construct provides appropriately located signals for transcription, translation, secretion and the like, including a signal peptide and an in-frame-AUG as required. Such vectors are described, for instance, in Luckow et al., (1989) Virology 170:31-39.

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described herein. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (e.g., GENECLEAN™ available from BIO 101 Inc., La Jolla, Calif., USA). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (e.g., GENECLEAN™ available from BIO 101 Inc., La Jolla, Calif., USA).

The fragment and the dephosphorylated plasmid are ligated together with T4 DNA ligase. E. coli HB101 or other suitable E. coli hosts such as XL-1 Blue (Stratagene Cloning Systems, La Jolla, Calif., USA) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transformed with 1.0 μg of a commercially available linearized baculovirus DNA (BACULOGOLD™ baculovirus DNA, Pharmingen, San Diego, Calif., USA), using the lipofection method described by Felgner et al., (1987) Proc. Natl. Acad. Sci. USA 84:7413-7417. One μg of BACULOGOLD™ virus DNA and 5 μg of the plasmid are mixed in a sterile well of a microtiter plate containing 50 μl of serum-free Grace's medium (Life Technologies Inc., Gaithersburg, Md., USA). Afterwards, 10 μl lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers et al. (Summers et al., “A Manual of Methods for Baculovirus Vectors and Insect Cell Culture Procedures” Texas Agricultural Experimental Station Bulletin No. 1555 (1987)). An agarose gel with BLUE GAL (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, Md., USA, page 9-10.) After appropriate incubation, blue stained plaques are picked with the tip of a micropipettor (e.g., an Eppendorf® micropipettor). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md., USA). After 42 hours, 5 μCi of ³⁵S-methionine and 5 μCi ³⁵S-cysteine (available from Amersham Biosciences, Piscataway, N.J., USA) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein can be used to determine the amino terminal sequence of the produced protein.

### Example 11

### Expression of a Polypeptide in Mammalian Cells

A polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Piscataway, N.J., USA), pRSVcat (ATCC 37152), pSV2dhfr (ATCC 37146), pBC12MI (ATCC 67109), pCMVSport 2.0, and pCMVSport 3.0. Mammalian host cells that could be used include, human HeLa, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest (See, e.g., Alt et al., (1978) J. Biol. Chem. 253:1357-1370; Hamlin & Ma, (1990) Biochem. Biophys. Acta 1097:107-143; Page & Sydenham, (1991) Biotechnology 9:64-68). Another useful selection marker is the enzyme glutanine synthase (GS) (Murphy et al., (1991) Biochem J. 227:277; Bebbington et al., (1992) Bio/Technology 10:169-175). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence (see, e.g., PCT Publication WO 96/34891). The amplified fragment is isolated from a 1% agarose gel using a commercially available kit (GENECLEAN® BIO 101 Inc., La Jolla, Calif., USA). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

In one example, Chinese hamster ovary cells lacking an active DHFR gene is used for transformation. Five μg of an expression plasmid is cotransformed with 0.5 μg of the plasmid pSVneo using lipofectin (Felgner et al., (1987) Proc. Natl. Acad. Sci. USA 84:7413-7417). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 12

### Production of an Antibody from a Polypeptide

The antibodies of the present invention can be prepared by a variety of methods (see, e.g., Current Protocols in Molecular Biology, (Ausubel et al., eds.), Greene Publishing Associates and Wiley-Interscience, New York (2002), Chapter 2, incorporated herein by reference in its entirety). As one example of such methods, cells expressing a polypeptide of the present invention are administered to an animal to induce the production of sera containing polyclonal antibodies. In a representative method, a preparation of the protein is prepared and purified to render it substantially free of natural contaminants. Such a preparation is then introduced into an animal in order to produce polyclonal antisera of greater specific activity.

In a preferred method, the antibodies of the present invention are monoclonal antibodies (or protein binding fragments thereof). Such monoclonal antibodies can be prepared using hybridoma technology. (Köhler et al., (1975) Nature 256:495; Köhler et al., (1976) Eur. J. Immunol. 6:511; Köhler et al., (1976) Eur. J. Immunol. 6:292; Hammerling et al., in: Monoclonal Antibodies and T-Cell Hybridomas, Elsevier, N.Y., pp. 563-681 (1981), incorporated herein by reference). In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Wands et al., (1981) Gastroenterology 80:225-232). The hybridoma cells obtained through such a selection are then assayed to identify clones that secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention can be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (see, e.g., Morrison, (1985) Science 229:1202; Oi et al., (1986) BioTechniques 4:214; U.S. Pat. No. 4,816,567; EP 171496; EP 173494; PCT Publications WO 86/01533 and WO 8702671; Boulianne et al., (1984) Nature 312:643; Neuberger et al., (1985) Nature 314:268, incorporated herein by reference).

Moreover, in another representative method, the antibodies directed against the polypeptides of the present invention can be produced in plants. Specific methods are disclosed in U.S. Pat. Nos. 5,959,177, and 6,080,560. These references not only describe methods of expressing antibodies, but also the means of assembling foreign multimeric proteins in plants (i.e., antibodies, etc,), and the subsequent secretion of such antibodies from the plant.

### Example 13

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

In one example, antibody-sandwich ELISAs are used to detect polypeptides in a sample, preferably a biological sample. Wells of a microtiter plate are coated with specific antibodies, at a final concentration of 0.2 to 10 μg/ml. The antibodies are either monoclonal or polyclonal and are produced by the method described elsewhere herein. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 14

### Method of Treatment Using Gene Therapy—Ex Vivo

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks. pMV-7 (Kirschmeier et al., (1988) DNA 7:219-25), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in the Examples herein or otherwise known in the art, using primers and having appropriate restriction sites and initiation/stop codons, if necessary. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagle's Medium (DMEM) with 10% calf serum (CS), penicillin and streptomycin. The vector containing the gene and any other desired sequence (e.g., a sequence from a murine sarcoma virus (MSV)) is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a MILLIPORE filter to remove detached producer cells and this media is then used to infect fibroblast cells. Media is removed from a sub-confluent plate of fibroblasts and quickly replaced with the media from the producer cells. This media is removed and replaced with fresh media If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on CYTODEX™ 3 microcarrier beads (Amersham Biosciences, Piscataway, N.J.).

### Example 15

### Method of Treatment Using Gene Therapy—In Vivo

Another aspect of the present invention comprises using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. A polynucleotide of the present invention may be operatively linked to a promoter or any other genetic elements necessary for the expression of the polypeptide by the target tissue. Such gene therapy and delivery techniques and methods are known in the art, (see, for example, PCT Publications WO 90/11092 and WO 98/11779; U.S. Pat. Nos. 5,693,622, 5,705,151, 5,580,859; Tabata et al., (1997) Cardiovasc. Res. 35(3):470-479; Chao et al., (1997) Pharmacol. Res. 35(6):517-522; Wolff, (1997) Neuromuscul. Disord. 7(5):314-318; Schwartz et al., (1996) Gene Ther. 3(5):405411; Tsurumi et al., (1996) Circulation 94 (12):3281-3290, incorporated herein by reference).

The polynucleotide constructs can be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner et al., (1995) Ann. NY Acad. Sci. 772:126-139 and Abdallah et al., (1995) Biol. Cell 85 (1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. A preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 μm cross-section of the individual quadriceps muscles is histochemically stained for protein expression. A time course for protein expression can be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 16

### Additional Methods of Genotyping the SNPs of the Present Invention

Those of ordinary skill in the art would acknowledge that there are a number of methods that may be employed for genotyping a SNP of the present invention, aside from the preferred methods described herein. The present invention encompasses the following non-limiting types of genotype assays: PCR-free genotyping methods, single-step homogeneous methods, homogeneous detection with fluorescence polarization, pyrosequencing, “tag” based DNA chip system, bead-based methods, fluorescent dye chemistry, mass spectrometry based genotyping assays, TaqMan genotype assays, invader genotype assays, and microfluidic genotype assays, among others.

Specifically encompassed by the present invention are the following, non-limiting genotyping methods: Landegren et al., (1998) Genome Res 8:769-776; Kwok, (2000) Pharmacogenomics 1:95-100; Gut, (2001) Hum Mutat 17:475-492; Whitcombe et al., (1998) Curr Opin Biotechnol. 9:602-608; Tillib & Mirzabekov, (2001) Curr. Opin. Biotechnol. 12:53-58; Winzeler et al., (1998) Science 281:1194-1197; Lyamichev et al., (1999) Nat. Biotechnol. 17:292-296; Hall et al., (2000) Proc. Natl. Acad. Sci. USA 97:8272-8277; Mein et al., (2000) Genome Res 10:333-343; Ohnishi et al., (2001) J. Hum. Genet. 46:471-477; Nilsson et al., (1994) Science 265:2085-2088; Baner et al., (1998) Nucleic Acid Res. 26:5073-5078; Baner et al., (2001) Curr. Opin. Biotechnol. 12:11-15; Hatch et al., (1999) Genet. Anal. 15:35-40; Lizardi et al., (1998) Nat. Genet. 19:225-232; Zhong et al., (2001) Proc. Natl. Acad. Sci. USA 98:3940-3945; Farugi et al., (2001) BMC Genomics 2:4; Livak, (1999) Genet. Anal. 14:143-149; Marras et al., (1999) Genet. Anal. 14:151-156; Ranade et al., (2001) Genome Res. 11:1262-1268; Myakishev et al., (2001) Genome Res. 11:163-169; Beaudet et al., (2001) Genome Res. 11:600-608; Chen et al., (1999) Genome Res. 9:492-498; Gibson et al., (1997) Clin. Chem. 43:1336-1341; Latif et al., (2001) Genome Res. 11:436-440; Hsu et al., (2001) Clin. Chem. 47:1373-1377; Alderborn et al., (2000) Genome Res. 10:1249-1258; Ronaghi et al., (1998) Science 281:363-365; Ronaghi, (2001) Genome Res. 11:3-11; Pease et al., (1994) Proc. Natl. Acad. Sci. USA 91:5022-5026; Southern et al., (1993) Genomics 13:1008-1017; Wang et al., (1998) Science 280:1077-1082; Brown & Botstein, (1999) Nat. Genet. 21:33-37; Cargill et al., (1999) Nat. Genet. 22:231-238; Dong et al., (2001) Genome Res. 11:1418-1424; Halushka et al., (1999) Nat. Genet. 22:239-247; Hacia, (1999) Nat. Genet. 21:42-47; Lipshutz et al., (1999) Nat. Genet. 21:20-24; Sapolsky et al., (1999) Genet. Anal. 14:187-192; Tsuchihashi. & Brown, (1994) J. Virol. 68: 5863; Herschlag, (1995) J. Biol. Chem. 270:20871-20874; Head et al., (1997) Nucleic Acid Res. 25:5065-5071; Nikiforov et al., (1994) Nucleic Acid Res. 22:4167-4175; Syvanen et al., (1992) Genomics 12:590-595; Shumaker et al., (1996) Hum. Mutat. 7:346-354; Lindroos et al., (2001) Nucleic Acids Res. 29:E69-9; Lindblad-Toh et al., (2000) Nat. Genet. 24:381-386; Pastinen et al., (2000) Genome Res. 10:1031-1042; Fan et al., (2000) Genome Res. 10:853-860 (2000); Hirschhorn et al., (2000) Proc. Natl. Acad. Sci. USA 97:12164-12169; Bouchie, (2001) Nat. Biotechnol. 19:704; Hensel et al., (1995) Science 269:400-403; Shoemaker et al., (1996) Nat. Genet. 14:450456; Gerry et al., (1999) J. Mol. Biol. 292:251-262; Ladner et al., (2001) Lab. Invest. 81:1079-1086; Iannone et al., (2000) Cytometry 39:131-140; Fulton et al., (1997) Clin. Chem. 43:1749-1756; Armstrong et al., (2000) Cytometry 40:102-108; Cai et al., (2000) Genomics 69:395; Chen et al., (2000) Genome Res. 10:549-557; Ye et al., (2001) Hum. Mutat. 17:305-316; Michael et al., (1998) Anal. Chem. 70:1242-1248; Steemers et al., (2000) Nat. Biotechnol. 18:91-94; Chan & Nie, (1998) Science 281:2016-2018; Han et al., (2001) Nat. Biotechnol. 19:631-635; Griffin & Smith, (2000) Trends Biotechnol. 18:77-84; Jackson et al., (2000) Mol. Med. Today 6:271-276; Haff & Smirnov, (1997) Genome Res. 7:378-388; Ross et al., (1998) Nat. Biotechnol. 16:1347-1351; Bray et al., (2001) Hum. Mutat. 17:296-304; Sauer et al., (2000) Nucleic Acids Res. 28:E13; Sauer et al., (2000) Nucleic Acid Res. 28:E100; Sun et al., (2000) Nucleic Acids Res. 28:E68; Tang et al., (1999) Proc. Natl. Acad. Sci. USA 91:10016-10020; Li et al., (1999) Electrophoresis 20:1258-1265; Little et al., (1997) Nat. Med. 3:1413-1416; Little et al., (1997) Anal. Chem. 69:4540-4546; Griffin et al., (1997) Nat. Biotechnol. 15:1368-1372; Ross et al., (1997) Anal. Chem. 69:4197-4202; Jiang-Baucom et al., (1997) Anal. Chem. 69:4894-4898; Griffin et al., (1999) Proc. Natl. Acad. Sci. USA 96:6301-6306; Kokoris et al., (2000) Mol. Diagn. 5:329-340; Jurinke, (2001); and/or Taranenko et al., (1996) Genet. Anal. 13:87-94, incorporated herein by reference.

### References

The entire disclosure of each document cited (including patents, patent applications, journal articles, abstracts, laboratory manuals, books, or other disclosures) referenced herein is hereby incorporated herein by reference. Further, the hard copy of the Sequence Listing submitted herewith and the corresponding computer readable form are both incorporated herein by reference in their entireties.

It will be clear that the invention may be practiced otherwise than as particularly described in the foregoing description and examples. Numerous modifications and variations of the present invention are possible in light of the above teachings and, therefore, are within the scope of the appended claims. Thus, various details of the present invention can be changed without departing from the scope of the invention. Furthermore, the foregoing description is for the purpose of illustration only and not for purposes of limitation.

1. An isolated nucleic acid molecule comprising a polymorphic site, wherein the nucleic acid molecule is SEQ ID NO:1 and the polymorphic site is nucleotide position 1415 wherein the reference nucleotide for said polymorphic sites is a guanidine at nucleotide position 1415, and wherein the nucleotide at the polymorphic sites in the isolated nucleic acid molecule is a nucleotide other than the reference nucleotide.

2. An isolated nucleic acid molecule according to claim 1, wherein the nucleotide other than the reference nucleotide is an adenine at nucleotide position 1415.

3. A portion of the isolated nucleic acid molecule of claim 1, wherein the portion has a length of at least 10 nucleotides.

4. A portion of the isolated nucleic acid molecule of claim 1, wherein the portion has a length of at least 20 nucleotides.

5. An oligonucleotide that hybridizes to the isolated nucleic acid molecule of claim 1 under stringent hybridization conditions.

6. The oligonucleotide of claim 5 that is a probe.

7. The oligonucleotide of claim 6, wherein a central nucleotide of the probe hybridizes with the polymorphic site of the portion of the nucleic acid molecule.

8. The oligonucleotide of claim 5 that is a primer.

9. A polypeptide comprising SEQ ID NO:4.

10. An isolated nucleic acid molecule comprising a polymorphic site, wherein the nucleic acid molecule is SEQ ID NO:1 and the polymorphic site is nucleotide position 1904 wherein the reference nucleotide for said polymorphic sites is a guanidine at nucleotide position 1904, and wherein the nucleotide at the polymorphic sites in the isolated nucleic acid molecule is a nucleotide other than the reference nucleotide.

11. The isolated nucleic acid molecule of claim 10, wherein the nucleotide other than the reference nucleotide is an adenine at nucleotide position 1904.

12. A portion of the isolated nucleic acid molecule of claim 10, wherein the portion has a length of at least 10 nucleotides.

13. A portion of the isolated nucleic acid molecule of claim 10, wherein the portion has a length of at least 20 nucleotides.

14. An oligonucleotide that hybridizes to the isolated nucleic acid molecule of claim 1 under stringent hybridization conditions.

15. The oligonucleotide of claim 14 that is a probe.

16. The oligonucleotide of claim 15, wherein a central nucleotide of the probe hybridizes with the polymorphic site of the portion of the nucleic acid molecule.

17. The oligonucleotide of claim 14 that is a primer.

18. A polypeptide comprising SEQ ID NO:6.

19. A method of analyzing a human nucleic acid sample comprising a nucleic acid molecule having a polymorphic site, wherein the nucleic acid molecule is SEQ ID NO:1 and the polymorphic site is selected from the group consisting of nucleotide position 1415 and nucleotide position 1904, wherein the reference nucleotide for said polymorphic sites is an adenine at nucleotide position 1415 and an adenine at nucleotide position 1904, and wherein the nucleotide at the polymorphic site in the isolated nucleic acid molecule is a nucleotide other than the reference nucleotide, the method comprising obtaining nucleic acid molecules from a nucleic acid sample and determining a nucleotide occupying one or more polymorphic sites of the nucleic acid molecule.

20. The method of claim 19, wherein the nucleotide other than the reference nucleotide is an adenine.

21. The method according to claim 19, wherein the nucleic acid sample is obtained from a plurality of individuals, and the nucleotide occupying one or more polymorphic sites is determined in each of the individuals, and wherein the method further comprises:
(a) testing each individual for the presence of a phenotype; and
(b) correlating the presence of the phenotype with the nucleotide occupying one or more polymorphic sites.

22. The method of claim 21, wherein the phenotype is selected from the group consisting of HDL levels elevated above normal human HDL levels, a decreased likelihood of cardiovascular disease and increased risk of lipid-related side-effects associated with the administration of a protease-inhibitor.

23. A method for predicting the likelihood that a human subject has, or is predisposed to, a condition selected from the group consisting of cardiovascular disease and lipid-related side-effects associated with the administration of a protease inhibitor, comprising:
determining the nucleotide present at a nucleotide position selected from the group consisting of 1415 and 1904, and of the SREB1 gene having the nucleotide sequence of SEQ ID NO:1 in a nucleic acid sample obtained from a human subject,
wherein the presence of an “A” (adenine) at the nucleotide position is indicative of a lower likelihood that the human subject has the condition than a human subject having a “G” (guanine) at the nucleotide position.

24. A method for predicting the likelihood that a human subject has, or is predisposed to, a condition selected from the group consisting of cardiovascular disease and lipid-related side-effects associated with the administration of a protease inhibitor, comprising:
determining the amino acid present at position selected from the group consisting of 417 and 580, and of a SREB1 polypeptide having the amino acid sequence of SEQ ID NO:2, the polypeptide being obtained from a human subject,
wherein the presence of a methionine residue at the amino acid position is indicative of a lower likelihood that the human subject has the condition than a human subject having a valine at the amino acid position.
